(19) United States
(12) Patent Application Publication
(10) Pub. No.: US 2026/0109967 A1
(43) Pub. Date: Apr. 23, 2026

(54) PSEUDO-VIRAL SYSTEMS FOR MUTATIONAL SCANNING OF VIRAL PROTEINS

(71) Applicant: Fred Hutchinson Cancer Center, Seattle, WA (US)

(72) Inventors: Bernadeta Dadonaite, Seattle, WA (US); Caelan Radford, Seattle, WA (US); Jesse Bloom, Seattle, WA (US); Katharine Dusenbury Crawford, Seattle, WA (US)

(73) Assignee: Fred Hutchinson Cancer Center, Seattle, WA (US)

(21) Appl. No.: 19/120,534

(22) PCT Filed: Oct. 12, 2023
(86) PCT No.: PCT/US2023/076730

§ 371 Date: Apr. 11, 2025

Related U.S. Application Data
(60) Provisional application No. 63/379,269, filed on Oct. 12, 2022.

(51) Int. Cl. C12N 15/10; G01N 33/50; G01N 33/68

(52) U.S. Cl. CPC C12N 15/1058; C12N 15/1055; G01N 33/5091; G01N 33/6845

(57) ABSTRACT

Cell-stored barcoded viral protein libraries with are described. The libraries can be used to map resistance mutations to therapeutic treatments. The libraries include features that allow efficient collection and assessment of informative data, obviating many bottlenecks of previous approaches.

### CROSS-REFERENCE TO RELATED APPLICATIONS

This application is a U.S. National Phase Patent Application based on International Patent Application No. PCT/US2023/076730, which claims priority to U.S. Provisional Patent Application No. 63/379,269 filed Oct. 12, 2022, both of which are incorporated herein by reference in their entirety as if fully set forth herein.

### STATEMENT OF GOVERNMENT SUPPORT

This invention was made with government support under A1141707, and A1140891 awarded by the National Institutes of Health. The government has certain rights in the invention.

### REFERENCE TO SEQUENCE LISTING

The Sequence Listing associated with this application is provided in xml format in lieu of a paper copy, and is hereby incorporated by reference into the specification. The name of the text file containing the Sequence Listing is 3ER4711.XML. The text is 32,435 bytes, was created on Apr. 10, 2025, and is being submitted electronically via Patent Center.

### FIELD OF THE DISCLOSURE

Cell-stored barcoded viral protein libraries are disclosed. Specifically, libraries that can be used to map resistance mutations to therapeutic treatments; predict viruses that may become resistant to therapeutic treatments and/or more easily evolve to infect new species; and to more safely study dangerous viruses that normally require high-safety biocontainment facilities are disclosed.

### BACKGROUND OF THE DISCLOSURE

While vaccination has all but eliminated smallpox and polio, the on-going mutation of other viruses continues to pose significant health threats. For example, there are sixty known influenza viruses and the predominance of any particular strain changes every year, requiring influenza vaccines to be continually updated to be effective. Other viruses such as human immunodeficiency virus (HIV), Ebola virus, and Middle Eastern respiratory syndrome coronavirus (MERS-CoV) also continue to pose significant health threats. To combat the spread of viruses, tools are needed to evaluate when drugs, vaccines, or antibodies are effectively working against viral proteins, or conversely, when viral proteins have or are likely to develop resistance to these countermeasures and pose a greater risk.

Proteins are made of strings of amino acids with different proteins having different numbers and orders of amino acids. Mutations in viral proteins allow viruses to continue to evolve and potentially increase virulence and develop resistance to treatments or vaccines. Altering amino acids at different positions through mutagenesis can help identify those amino acids that are essential to the function of the protein and provide an understanding of the impact of mutations on drug resistance, immune escape, vaccination efficacy, and pathogenesis. Another tool in assessing viral function is deep mutational scanning which uses high-throughput screening to assess the function of a large number of protein variants.

Replication of retroviruses such as lentiviruses, a type of virus that has an RNA genome, has been well studied. Once a retrovirus gains entry into a host cell, the viral RNA genome is copied by specialized enzymes into a DNA form that then goes to the nucleus of the host cell, where the host cell genome resides. The viral DNA integrates itself into the host cell genome. The ends of the viral RNA genome are flanked by regions of sequences called long terminal repeats (LTRs), which facilitate this integration. A region of the LTR called the U3 is important for transcription and packaging of the viral RNA genome (vRNA). After synthesis of viral gRNA, it is exported out of the nucleus into the host cell cytoplasm where this vRNA is packaged into new virions. After assembly and maturation of the nucleocapsid, the new virions exit the cell in a variety of ways. They may exit through budding in which part of the host cell membrane becomes part of the virus and breaks off from the cell, exocytosis in which substances are secreted through the host cell membrane, or lysis, in which the cell membrane is ruptured. Once the viruses have exited the cell, they continue to spread.

In the context of viral infection, years of research have led to an understanding of many of the proteins important in the virus life cycle. A virion is a complete infective form of a virus outside of a host's cell. The first step in infecting cells is binding of the virion's viral entry protein to a host cell. This binding is followed by fusion of the virion with the host cell and transfer of the viral DNA or RNA into the host cells. Once the viral DNA or RNA enters the host's cells, viruses begin to multiply using the host's ribosomes to generate viral proteins.

For many human pathogenic viruses, the binding and fusion steps are performed by a single viral entry protein. For example, influenza virus, HIV, Ebola virus, and Lassa virus, all use a single entry protein for binding and fusion with a host cell. For other viruses, multiple proteins are involved. For example, Nipah virus has separate binding and fusion proteins.

Viral entry proteins are a primary target of immune system responses against infection. Most vaccines elicit neutralizing antibodies to the viral entry protein. Therapeutic antibodies can also be used to impair the activity of viral entry proteins, with the potential to both protect against infection as well as therapeutically treat active infection. However, viral entry proteins are able to mutate and evolve, and mutations can allow these proteins to escape recognition by immune system responses and therapeutic antibodies.

A virus's viral entry protein is also a key determinant of the species that a particular virus can infect, and adaptive evolution of these entry proteins has been retrospectively characterized in most molecularly documented examples of non-human viruses jumping into humans. For example, the influenza pandemics of 1918, 1957, and 1968 all involved mutations that turned viral entry proteins from avian viral strains to strains that could better infect humans. The severe acute respiratory syndrome (SARS) coronavirus outbreak in 2003 was associated with mutations in the virus's entry protein that enabled it to better bind human receptors. The MERS-CoV viral entry protein also has mutations that increase binding to human cells. Recent evidence suggests that during the 2014-2016 Ebola outbreak, Ebola's entry protein acquired mutations that promoted infection of human cells. Comparing the growth of viral mutants in different cell types can serve to identify mutations that contribute to host adaptation.

As viral entry proteins are a primary target of immune system responses, mapping functional and antigenic effects of mutations of the entry proteins plays a role in the design of therapeutic agents and vaccines. The entry proteins of a few viruses (e.g., influenza, HIV) are well-characterized, but surprisingly little is known about the entry proteins of many less-studied viruses in part because these proteins are challenging to study. They form large metastable oligomers that are often heavily modified with sugar molecules which render them difficult targets for biochemistry and structural biology.

WO2020/006494 describes an approach for performing deep mutational scanning of proteins by providing cell-stored barcoded mutational scanning libraries of proteins. Among many potential uses, the described libraries can be used to quickly map with high resolution amino acid changes in a given protein that are important to escape binding to a ligand. The libraries can be used to predict viruses that may become resistant to therapeutic treatments and/or that may more easily evolve to infect new species. The libraries can also be used to more safely study dangerous viruses that normally require high-safety biocontainment facilities. The libraries include features that allow efficient collection and assessment of informative data, obviating many bottlenecks of previous approaches.

WO2020/006494 describes storage of a library of genes encoding variant proteins in a non-infective state within holding cells. More particularly, the library is stored as barcoded non-replicative variants inside cells. Virion production can be induced by transfecting the storing cells with viral helper plasmids that encode the rest of the retroviral particle proteins. This results in expression in each cell of retroviral particles that are packaged with a barcoded gene encoding a given mutant viral entry protein and pseudotyped with that particular mutant viral entry protein. The ability to produce virions that package a barcoded gene encoding a mutant viral entry protein following transfection with helper plasmids is achieved, in part, through the use of a vector that is not self-inactivating. In particular embodiments, this is achieved by including a functional U3.

Following generation of virions from the storage cells, functional studies can be conducted to assess variant proteins. While the libraries described in WO2020/006494 provide several important advances, opportunities to further improve the libraries remain.

### SUMMARY OF THE DISCLOSURE

The current disclosure provides modifications and new uses of the libraries described in WO2020/006494. The modifications further improve the WO2020/006494 libraries.

One modification of the current disclosure is in the design of viral backbones, operably connecting the variant protein of study to a particular inducible promoter and operably connecting a reporter and resistance gene to a different promoter. For example, the variant protein can be operably connected to the inducible rtTA promoter, while the reporter and resistance gene such as ZsGreen linked to puromycin resistance (PuR) via a T2A linker, are operably connected to a constitutive CMV promoter.

Another modification from the earlier libraries creates an environment permissive to variant protein expression and/or viral/target cell interaction by: (i) providing or enhancing pro-viral factors in producer cells; (ii) removing or otherwise inhibiting anti-viral factors from producer cells; (iii) providing or enhancing pro-viral factors in target cells; and/or (iv) removing or otherwise inhibiting anti-viral factors from target cells.

In certain instances, it may also be helpful to create an anti-viral environment. In these embodiments, an environment less permissive to variant protein expression and/or viral/target cell interaction can be created by: (i) providing or enhancing anti-viral factors in producer cells; (ii) removing or otherwise inhibiting pro-viral factors from producer cells; (iii) providing or enhancing anti-viral factors in target cells; and/or (iv) removing or otherwise inhibiting pro-viral factors from target cells.

Another modification includes using spike in controls for reference levels based on proteins that are not subject to pre-existing immunity in humans. The controls can be formed in parallel with the construction of the libraries.

Other modifications are described elsewhere herein. Each of the disclosed modifications can be practiced individually or in combination with other modifications described herein.

### BRIEF DESCRIPTION OF THE SEVERAL VIEWS OF THE DRAWINGS

Some of the drawings submitted herein may be better understood in color. Applicant considers the color versions of the drawings as part of the original submission and reserves the right to present color images of the drawings in later proceedings.

FIGS. 1A-1F. Published in WO2020/006494. Exemplary lentiviral backbone constructs. (FIG. 1A) Each viral entry protein variant can be barcoded with 18 nucleotides after the stop codon. Integration is marked by a GFP reporter with a T2A linker (Liu, et al. (2017) Scientific Reports 7: 2193) to the entry protein, and LTRs have the full, functional U3, meaning that integrated backbones, which include the barcode, can be transcribed and packaged into virions. (FIG. 1B) A lentiviral construct as in (FIG. 1A), but with the Woodchuck Hepatitis Virus Posttranscriptional Regulatory Element (WPRE) sequence removed to reduce the expression of the viral entry protein variant. (FIG. 1C) A lentiviral construct as in (FIG. 1A), but with a degradation domain linked to the viral entry protein variant to allow for the controlled expression of the viral entry protein. (FIG. 1D) A lentiviral construct as in (FIG. 1A), but with the viral entry protein variant under the control of an inducible promoter (here, the Tet-On® 3G system (Clontech Laboratories, Mountain View, CA) that is different than the promoter of the selectable marker. The Tet-On® 3G transactivator that activates the TRE3GS promoter when bound to an inducer molecule is constitutively expressed from the promoter that also controls the expression of the selectable marker. (FIG. 1E) A lentiviral construct as in (FIG. 1D), but without constitutive expression of the Tet-On® 3G transactivator necessary for induction (the transactivator would need to be supplied in trans). (FIG. 1F) A lentiviral construct as in (FIG. 1A), but with the viral entry protein variant and selectable marker under the control of an inducible promoter. RRE=Rev responsive element.

FIG. 2. Published in WO2020/006494. Process to create cells that store a library of viral entry protein variants (outlined in black box, VII). The viral vectors of I are the constructs depicted in FIG. 1A, lentiviral vectors including full functional U3, CMV promoter, GFP, 2A linker, and barcoded entry protein. The reporter (e.g., GFP) is marked with a *, and +, x, #, and \hat{ } indicate unique barcodes in I. Transfection of 293T cells (IV) with I, VSV G entry protein for the next round of infection (II), and plasmids encoding non-surface proteins for lentivirus production (Ill) produces VSV G pseudotyped lentiviral vectors encoding entry protein library with scrambled barcodes and no genotype-phenotype link (V). Low MOI infection of 293T cells leads to integration of at most a single copy of the vector encoding one entry protein variant (VI). 5×10⁵ unique GFP+ cells are sorted, and these cells store the library (VII). Transfection of cells in the library (VII) with helper plasmids (VIII) creates a lentiviral virion library with entry proteins linked to barcodes (IX).

FIGS. 3A-3F. Deep mutational scanning platform for spike, (FIG. 3A) traditional lentivirus backbone without spike gene. (FIG. 3B) Lentivirus backbone used for deep mutational scanning. The backbone contains functional lentiviral 5′ and 3′ long terminal repeat (LTR) regions. The spike gene is under an inducible tet response element 3rd generation (TRE3G) promoter, and there is a 16 nucleotide barcode (BC) downstream of the stop codon. A CMV promoter drives expression of reporter ZsGreen gene that is linked to a puromycin resistance gene (PuR) via a T2A linker. The backbone also contains a woodchuck hepatitis virus posttranscriptional regulatory element (WPRE), a Rev response element (RRE), and a central polypurine tract (cPPT). (FIG. 3C) Approach for creating genotype-phenotype linked lentivirus libraries. HEK-293T cells are transfected with spike carrying lentivirus backbone, VSV G expression plasmid, and lentiviral helper plasmids to generate VSV G pseudotyped lentiviruses. These viruses are used to transduce reverse tetracycline-controlled transactivator (rtTA) expressing HEK-293T cells at low multiplicity of infection (MOI), and successfully transduced cells are selected using puromycin. Selected cells can be transfected with helper plasmids and a VSV G expression plasmid to produce VSV G pseudotyped viruses carrying all genomes present in the deep mutational library or selected cells can be induced with doxycycline (dox) to express spike and transfected with only the helper plasmids to generate spike-pseudotyped lentiviruses that have a genotype-phenotype link. (3D) Average number of mutations per barcoded spike in BA.1 libraries. (FIG. 3E) Total number of barcoded variants in each BA.1 library. (3F) The coverage of intended mutations across all BA.1 libraries.

FIGS. 4A, 4B. Pseudovirus titers from phenotype-genotype linked lentiviruses. (FIG. 4A) Delta spike pseudotyped lentivirus titers. Viruses were produced under indicated conditions from cells with integrated lentivirus genomes carrying Delta spike. Virus titers for conditions used to generate the actual deep mutational scanning libraries are the leftmost dot on FIG. 4A. Viruses were titrated on ACE2-TMPRSS2-HEK-293T cells. (FIG. 4B) BA.1 or Delta spike-pseudotyped lentivirus titers in the presence or absence of amphotericin B (amphoB). BA.1 virus was titrated on ACE2-HEK-293T cells and Delta virus was titrated on ACE2-TMPRSS2-HEK-293T cells.

FIGS. 5A-5D. Delta spike deep mutational scanning libraries. (FIG. 5A) Total number of barcoded variants in each Delta library. (FIG. 5B) Coverage of intended mutations across both Delta libraries. (FIG. 5C) Average number of mutations per barcoded spike in Delta libraries. (FIG. 5D) Distribution of functional scores for variants with different types of mutations in the Delta libraries.

FIGS. 6A, 6B. Distribution of amino-acid mutations per variant in BA.1 (FIG. A) and Delta (FIG. B) deep mutational scanning libraries.

FIG. 7. Some types of mutations tend to impair spike-mediated pseudovirus infection. For each barcoded spike variant, a functional score was computed that reflects how well that spike mediates pseudovirus infection relative to the unmutated spike: negative scores indicate impaired infection and positive scores indicate improved infection. The plots show the distribution of functional scores across all variants in each of the three BA.1 libraries for different categories of variants, with each distribution colored by the mean functional score for that variant type.

FIGS. 8A-8D. A VSV G standard enables the measurement of absolute neutralization by deep sequencing. (FIG. 8A) Neutralization assay demonstrating that BA.1-spike-pseudotyped lentivirus is neutralized by antibody Ly-CoV1404, but the VSV G-pseudotyped neutralization standard is not. (FIG. 8B) Use of the VSV G standard to measure absolute neutralization. Deep mutational scanning libraries are mixed with VSV G neutralization standard. The virus mixture is incubated with a no-antibody control or increasing antibody concentrations and infected into ACE2-expressing 293T cells. After 12 hours viral genomes are recovered, barcodes are sequenced, and absolute neutralization of each variant is computed by comparing its barcode counts to those from the VSV G standard. (FIG. 8C) Fraction of barcodes derived from the VSV G neutralization standard in infections with increasing Ly-CoV1404 concentrations. (FIG. 8D) BA.1 deep mutational scanning library non-neutralized fractions averaged across variants with different numbers of amino-acid mutations at different Ly-CoV1404 concentrations. Note FIGS. 8C and 8D use a symlog scale.

FIG. 9. The VSV G neutralization standard is not neutralized by antibodies 5-7, CC9.104 and CC65.105. Neutralization assays using NTD-targeting 5-7 mAb and S2-targeting CC9.104 and CC65.105 antibodies against lentivirus pseudotyped with BA.1 spike or VSV G.

FIGS. 10A-10F. Antibody Ly-CoV1404 escape mapping. (FIG. 10A) Correlation of mutation escape scores between technical replicates (BA1 Lib-1.1 and BA1 Lib-1.2) and biological replicates (BA1 Lib-1, BA1 Lib-2, BA1 Lib-3). (FIG. 10B) Total escape scores at each site in the BA.1 spike, and a zoomed-in plot showing the key escape sites. Sites of mutations chosen for validation experiments are labeled on the x-axis. (FIG. 10C) Heatmap of mutation escape scores at key sites. Residues marked with X are the wild-type amino acids in BA.1. Amino acids not present in the libraries are shown in gray. (FIG. 10D) Surface representation of spike colored by sum of escape scores at that site. Ly-CoV1404 antibody is in yellow. Only the antibody-bound protomer is colored. PDB IDs 7MMO and 6XM4 were aligned to make this structure. (FIG. 10E) Validation pseudovirus neutralization assays of the indicated BA.1 spike mutants against the Ly-CoV1404 antibody. (FIG. 10F) Correlation between predicted IC₅₀ values from deep mutational scanning (DMS) data versus the IC₅₀ values measured in the validation assays in FIG. 10E. The points are colored as in FIG. 10E. Lower bound indicates that the antibody did not neutralize at the highest concentration tested in the validation neutralization assay. Site numbering in all plots is based on the Wuhan-Hu-1 sequence.

FIGS. 11A, 11B. Comparison between LY-CoV1404 escape mapping using full spike pseudovirus deep mutational scanning versus the previously described yeast-display deep mutational scanning of just the RBD. (FIG. 11A) Correlation between measured mutation-level escape scores for Ly-CoV1404 antibody in pseudovirus and yeast display deep mutational scanning experiments. Yeast display data is taken from (Starr et al., 2022, doi.org/10.1101/2022.09.20.508745). (FIG. 11B) Surface representation of SARS-CoV-2 RBD colored by sum of escape scores at that site. PDB ID: 6XM4.

FIGS. 12A-12E. Antibody 5-7 escape mapping. (FIG. 12A) Total escape scores for each site in the BA.1 spike and a zoomed-in plot showing the key escape sites. (FIG. 12E) Heatmap of mutation escape scores at key sites. Residues marked with X are the wild-type amino acids in BA.1. Amino acids not present in the libraries are shown in gray. (FIG. 12C) Surface representation of spike colored by the sum of escape scores at that site. Antibody 5-7 is shown in yellow in the inset. PDB ID: 7RW2. (FIG. 12D) Validation pseudovirus neutralization assays of the indicated BA.1 spike mutants against antibody 5-7. (FIG. 12E) Correlation between predicted IC₅₀ values from deep mutational scanning (DMS) data versus the IC₅₀ values measured in the validation assays in (FIG. 12F). The lower bound indicates that the antibody did not neutralize at the highest concentration tested in the validation neutralization assay. Site numbering in all plots is based on the Wuhan-Hu-1 sequence.

FIGS. 13A-13H. Antibody CC9.104 and CC67.105 escape mapping. (FIG. 13A, FIG. 13E) Total escape scores for each site in the BA.1 spike for the CC9.104 (FIG. 13A) and CC67.105 (FIG. 13E) antibodies. (FIG. 13C, FIG. 13D) Escape heatmaps for the S2 stem-helix (sites 1146-1163) for CC9.104 (FIG. 13C) and CC67.105 (FIG. 13D) antibodies. Residues marked with X are the wild-type amino acids in the BA.1 sequence. Amino acids that are not present in the libraries are shown in gray. (FIG. 13E) Surface representation of spike colored by the sum of escape scores at that site for CC9.104 (left) and CC67.105 (right) antibodies. Site 1163 is not resolved in the structure. PDB ID: 6XR8. (FIG. 13F) Alignment of SARS-CoV-2 and MERS-CoV spikes at sites 1146-1163. (FIG. 13G) Validation pseudovirus neutralization assay for CC9.104 (left) and CC67.105 (right) antibodies with BA.1 spike carrying the indicated mutations. (FIG. 13H) Correlation between predicted IC₅₀ values from deep mutational scanning (DMS) data versus the IC₅₀ values measured in the validation assays in (13G). The lower bound indicates that the antibody did not neutralize at the highest concentration tested in the validation neutralization assay. Site numbering in all plots is based on the Wuhan-Hu-1 sequence.

FIGS. 14A-14C. Antibody REGN10933 escape mapping using Delta deep mutational scanning libraries (FIG. 14A) Total escape scores for each site within Delta spike and a zoomed-in plot showing key escape sites. (FIG. 14E) Heatmap of mutation escape scores at key sites. Residues marked with X are the wild-type amino acids in the Delta sequence. Amino acids not present in the libraries are shown in gray. (FIG. 14C) Surface representation of spike colored by sum of escape scores at that site. REGN10933 antibody is shown in green. PDB structures 6XDG and 6XM4 were aligned to make this figure. Site numbering in all plots is based on the Wuhan-Hu-1 sequence.

FIGS. 15A-15D. Functional effects of mutations on spike-mediated pseudovirus infection (FIG. 15A) Distribution of functional effects of mutations in BA.1 deep mutational scanning libraries. Negative values indicate mutations are deleterious for viral entry. The stop codon mutation with a neutral functional effect of 0 is at the last codon of the spike used in the experiments. (FIG. 15E) Heatmap showing functional effects at sites of mutations with beneficial functional effects that were chosen for validation assays in 15C. (FIG. 15C) Fold change in virus entry titer for spike mutants relative to unmutated spike. There are three points for each mutant, reflecting biological triplicate measurements. (FIG. 15D) Correlation between enrichment of mutations during the actual evolution of human SARS-CoV-2 and functional effects from the lentivirus-based deep mutational scanning or previously reported RBD expression or ACE2 affinity for yeast-based RBD deep mutational scanning (Starr et al., 2022, doi.org/10.1101/2022.09.20.508745), and S2 (Tan et al., 2022, bioRxiv 2022.09.24.509341) or NTD (Ouyang et al., 2022, bioRxiv 2022.06.20.496903) expression for mammalian display-based deep mutational scanning.

FIG. 16. Primer sequences used for building deep mutational scanning libraries.

FIG. 17. Depiction of membrane-bound viral proteins. Ecd is the ectodomain, TM is the transmembrane domain, and CTD is the cytoplasmic tail.

FIG. 18A, 18B. Heatmaps of mutation-escape scores at key sites for Delta breakthrough sera 267C (FIG. 18A) and 279C (FIG. 18E); residues marked with X are wild-type amino acids in the Delta sequence.

FIG. 19A, 19B. Validation pseudovirus neutralization assays of the indicated Delta spike mutants against the Delta breakthrough sera. Error bars indicate standard error between two technical replicates.

FIG. 20A, 20B. Correlation between predicted IC₅₀ values from DMS data versus IC₅₀ values measured in the validation assays. Site numbering in all plots is based on the Wuhan-Hu-1 sequence. Site 452 likely contains many sensitizing mutations because it is mutated in Delta relative to the original vaccine received by the individuals from which the sera is derived.

FIGS. 21A, 21B. Lentivirus platform for deep mutational scanning. (FIG. 21A) The lentivirus genome used for deep mutational scanning. The genome contains the full 50 and 30 LTR sequences, including the U3 sequence usually deleted in the 30 LTR. Env is under the control of an inducible TRE3G promoter and followed by a 16N random nucleotide barcode. A CMV promoter drives ZsGreen and puromycin resistance (PuR) expression. (FIG. 21B) Approach for generating genotype-phenotype linked variant libraries. Lentivirus genomes carrying barcoded Env mutants are transfected into 293T cells alongside plasmids expressing the lentiviral proteins necessary for creating single-cycle infectious virions and VSV G. The resulting VSV G pseudotyped viruses are used to infect 293T-rtTA cells at a low multiplicity of infection, such that most infected cells receive just one viral genome. Infected cells are enriched via puromycin selection, and genotype-phenotype-linked Env-expressing virus variant libraries are generated by inducing Env expression with doxycycline and transfecting plasmids encoding the lentivirus genes. The virus variant libraries are also generated separately with VSV G, and these VSV G pseudotyped viruses can infect cells regardless of whether or not they have a functional Env and so can be used to readout the library composition.

FIGS. 22A-22E. Mutant library design and functional effects of mutations. (FIG. 22A) Choice of targeted mutations based on measured effects in prior deep mutational scanning (Haddox, H. K., et al. Elife 7. E34420) and occurrences in natural HIV sequences. The distributions of previously measured mutation effects are shown for all mutations to BF520 (black) with highlighting of subsets of mutations (grey). From left to right, highlighted are mutations well tolerated in the prior deep mutational scanning, (Haddox, H. K., et al. supra) mutations found multiple times in natural sequences, and the union of these two sets. Mutations in the union of the two sets were designed into the disclosed libraries. Functional effect of mutation in previous deep mutational scanning by (Haddox, H. K., et al. supra). (FIG. 22B) Average codon mutations per Env mutant, separated by type of codon mutation. (FIG. 22C) Total number of barcoded Env mutants in each library, along with the numbers of unique mutations and percentage of the intended mutations present across these mutants. (FIG. 22D) Distributions of functional scores measured in deep mutational scanning across Env mutants, separated by the types of codon mutations found in the mutants. Negative functional scores indicate impaired Env-mediated infection relative to unmutated BF520 Env. Histograms are colored by mean functional score. (22E) Distributions of mutation effects versus how often that mutation is found in natural sequences. The distribution of stop codon effects is also shown.

FIGS. 23A-23E. Neutralization escape map for antibody PGT151. (FIG. 23A) The top panel shows PGT151 escape across all sites in the BF520 Env ectodomain, and the bottom panel zooms into key sites. The y-axis shows escape summed across all mutations at each site. (FIG. 23B) Heatmap of effects of individual mutations at key sites of escape. Residues marked with X are wild-type residues in BF520. Residues grayed out are not present in the variant libraries, typically because they are deleterious for Env function. See dms-vep.github.io/HIV_Envelope_BF520_DMS_CD4bs_sera/PGT151_escape_plot.html for an interactive version of the site and mutation escape plots. (FIG. 23C) Site escape mapped onto a structure of PGT151-bound Env, with red indicating sites where mutations cause escape. Residues within 4 angstroms of antibody PGT151 in the structure are outlined in black. Glycans are colored yellow. This visualization was generated using the structure of BG505 DCT N332T (PDB 6MAR, antibody PGT151 removed). (FIG. 23D) Correlation of predicted fold-change in IC50 from the current deep mutational scanning versus fold change in IC50 as previously measured in TZM-bl neutralization assays. (Dingens, et al., Cell Host Microbe 21, 777-787. e4.) Mutations are colored by the max neutralization plateau observed for that variant in TZM-bl neutralization assays using PGT151. Pearson correlation coefficients were calculated on the fold changes on a linear scale. Note the fold change IC50 predicted by deep mutational scanning is calculated using the full biophysical model fit to the data (see STAR Methods). (23E) Correlation of fold enrichment in prior HIV Env deep mutational scanning (Dingens, et al., supra) versus fold-change in IC50 for the same set of single mutants.

FIGS. 24A-24E. Neutralization escape map for antibody 1-18 and purified IgGs from IDC561. (FIG. 24A) Escape at all sites in the BF520 Env ectodomain from antibody 1-18 and serum IDC561. Positive values represent sites where mutations cause escape on average, whereas negative values represent sites where mutations enhance neutralization on average. See dms-vep.github.io/HIV_Envelope_BF520_DMS_CD4bs_sera/1-18_escape_plot.html and dms-vep.github.io/HIV_Envelope_BF520_DMS_CD4bs_sera/IDC561_escape_plot.html for interactive versions of the escape maps for 1-18 and IDC561, respectively. (FIG. 24B) Site escape from 1-18 mapped onto a structure of 1-18-bound Env. Residues within 4 angstroms of antibody 1-18 in the structure are outlined in black. This visualization was generated using the structure of BG505.SOSIP.664 (PDB 6UDJ, antibodies 10-1074 and 1-18 removed). (Schommers, et al., Cell 180, 471-489.e22.) (FIG. 24C) Site escape from IDC561 mapped onto the same structure. (FIG. 24D) Scatter plot of how mutations escape serum IDC561 versus antibody 1-18. (FIG. 24E) Correlations of how mutations escape IDC561 versus antibodies 1-18, 3BNC117, or PGT151.

FIGS. 25A-25E. Neutralization escape maps for antibody 3BNC117 and purified IgGs from IDC513 (FIG. 25A) Escape at all sites in BF520 Env ectodomain from antibody 3BNC117 and serum IDC513. See dms-vep.github.io/HIV_Envelope_BF520_DMS_CD4bs_sera/3BNC117_escape_plot.html and dms-vep.github.io/HIV_Envelope_BF520_DMS_CD4bs_sera/IDC513_escape_plot.html for interactive versions of the escape maps for 3BNC117 and IDC513, respectively. (FIG. 25B) Site escape from 3BNC117 mapped onto a structure of 3BNC117-bound Env. Residues within 4 angstroms of antibody 3BNC117 in the structure are outlined in black. This visualization was generated using the structure of BG505.SOSIP.664 along with antibody 3BNC117 (PDB 5V8M). (Lee, et al., Immunity 46, 690-702.) (FIG. 25C) Site escape from IDC513 mapped onto the same structure. (FIG. 25D) Scatter plot of how mutations escape serum IDC513 versus antibody 3BNC117. (FIG. 25E) Correlations of how mutations escape IDC513 versus antibodies 1-18, 3BNC117, or PGT151.

FIGS. 26A-26D. Neutralization escape maps for purified IgGs from IDF033 and IDC508 (FIG. 26A) Escape at all sites in BF520 Env ectodomain from serum IDF033 and serum IDC508. See dms-vep.github.io/HIV_Envelope_BF520_DMS_CD4bs_sera/IDF033_escape_plot.html and dms-vep.github.io/HIV_Envelope_BF520_DMS_CD4bs_sera/IDC508_escape_plot.html for interactive versions of the escape maps for IDF033 and IDC508, respectively. (FIG. 26B) Site escape from IDF033 mapped onto a structure of Env. This visualization was generated using the structure of BG505.SOSIP.664 (PDB 6UDJ, antibodies 10-1074 and 1-18 removed). (Schommers, et al., Cell 180, 471-489.e22.) (FIG. 26C) Site escape from the first IDC508 epitope mapped onto the same structure. (FIG. 26D) Site escape from the second IDC508 epitope mapped onto the same structure.

FIGS. 27A-27D. Pseudovirus neutralization assays to validate deep mutational scanning measurements. (FIG. 27A) Neutralization curves for unmutated BF520 Env and single mutants against purified IgGs from each serum. (FIG. 27B) Correlation of deep mutational scanning predicted fold change IC80s for single mutants versus fold change in IC80 measured in the neutralization assay. R indicates the Pearson correlation. (FIG. 27C) Neutralization curves for combinations of mutations (and the constituent individual mutations repeated from (FIG. 27A). (FIG. 27D) Correlation of deep mutational scanning predicted fold change IC80s versus fold change IC80s measured in neutralization assays. Points in the scatter plots with asterisks overlap; therefore, they have been slightly jittered for clarity.

FIG. 28. Titers of Env or VSV G pseudotyped lentiviruses on TZM-bl cells, related to FIGS. 21A, 21B. Lentiviruses were produced by transfecting 293T cells with the same ZsGreen reporter lentiviral backbone, Rev, and Tat expressing plasmids for each condition, along with an HIV Env from the indicated viral strain or a VSV G expressing plasmid, and either an NL4-3 or NL4-3 V34I Q26R Gag-Pol expressing plasmid as indicated. Data are from different virus preparations and titering dates. The infectious units per mL on TZM-bl cells were normalized to VSV G infectious units per mL by dividing each condition's infectious units per mL by that of the VSV G pseudotyped virus with the same Gag-Pol that was produced and titered on the same dates, performing this normalization to correct for batch effects. The titers for the VS G pseudotyped viruses ranged from 1.5-35 million infectious units per mL. Gag-Pol mutations V34I and Q62R were made based on previous studies that showed these mutations can rescue Env incorporation deficiencies (Freed, et al., J. Virol. 70, 341-351; Tedbury, et al., PLoS Pathog. 9. e1003739).

FIGS. 29A-29C. Broadly neutralizing human anti-HIV sera, related to FIGS. 24A-24E, 25A-25E, 26A-26D, and 27A-27D. (FIG. 29A) Neutralization of a global HIV panel by each serum (deCamp, et al., J. Virol. 88, 2489-2507). Values reported are IC50 in ug/mL for purified IgGs. (FIG. 29B) Clinical data related to the individual from whom each serum was collected. (FIG. 29C) f61 neutralization fingerprinting results for each serum (Doria-Rose, et al., PLoS Pathog. 13. e1006148).

FIG. 30A, FIG. 30B. Zoomed in views of mutation-level escape at some key sites for IDF033 and IDC508, related to FIGS. 26A-26D. (FIG. 30A) Heatmap of escape of individual mutations in β23-V5-β24 for IDF033. Residues marked with X are wild-type residues in BF520. Residues grayed out are not present in the variant libraries. (FIG. 30B) Heatmap for IDC508 epitope 1. See dmsvep.github.io/HIV_Envelope_BF520_DMS_CD4bs_sera/IDF033_escape_plot.html and dmsvep.github.io/HIV_Envelope_BF520_DMS_CD4bs_sera/IDC508_escape_plot.html for interactive versions of the full Env escape maps for IDF033 and IDC508, respectively.

### DETAILED DESCRIPTION

Proteins are made of strings of amino acids with different proteins having different numbers and orders of amino acids. Proteins are essential to the functioning of cells and organisms. A powerful way to study proteins is through mutagenesis. Mutagenesis refers to altering the amino acid that naturally occurs at a position along the string of amino acids that create a given protein. Systematically altering amino acids at different positions through mutagenesis can identify those amino acids that are essential to the function of the protein. Deep mutational scanning refers to methods of generating and characterizing hundreds of thousands of mutants or more of a given protein. More particularly, deep mutational scanning can refer to altering each amino acid position with all possible alternative amino acids. More particularly, deep mutational scanning can refer to altering each amino acid position with all possible alternative amino acids.

One scenario where the study of proteins is extremely beneficial is in relation to viruses. Many viruses can be effectively managed or treated. For example, vaccination has all but ameliorated smallpox and measles, once among mankind's greatest scourges. Unfortunately, however, numerous viruses continue to pose significant health threats. Examples include severe acute respiratory syndrome coronavirus (SARS-CoV), SARS-CoV-2, Middle Eastern respiratory syndrome coronavirus (MERS-CoV), influenza, human immunodeficiency virus (HIV), and Ebola virus.

To combat the spread of viruses, scientists and doctors need tools to know when therapeutic treatments (e.g., drugs, vaccines, or antibodies) are working against viral proteins, or conversely, when these viral proteins have developed resistance to therapeutics and pose a greater risk.

Replication of retroviruses such as lentiviruses, a type of virus that has an RNA genome, has been well studied. Once a retrovirus gains entry into a host cell, the viral RNA genome is copied by specialized enzymes into a DNA form that then goes to the nucleus of the host cell, where the host cell genome resides. The viral DNA integrates itself into the host cell genome. The ends of the viral RNA genome are flanked by regions of sequences called long terminal repeats (LTRs), which facilitate this integration, along with the virion integrase. The number of possible sites of integration into the cellular genome is very large and widely distributed. Cellular enzymes are used for replication of the integrated viral DNA in concert with cellular chromosomal DNA, and cellular RNA polymerase II is used for expression of the integrated viral DNA. A region of the LTR called the U3 is important for a process called transcription, where the integrated DNA form of the retrovirus is converted back to a messenger RNA (mRNA) form. After synthesis of viral mRNA, the mRNA is exported out of the nucleus into the host cell cytoplasm where this mRNA can be used in a process called translation to produce more of the viral proteins that then are assembled along with the retroviral genome into new virions. The new virions bud off from the cell to start a new cycle of infection.

In the context of viral infection, years of research have led to an understanding of many of the proteins important in the virus life cycle. A virion is a complete infective form of a virus outside of a host's cell. The first step in viral infection is the binding of the virion's viral entry protein to a host cell. This binding is followed by fusion of the virion with the host cell. For many human pathogenic viruses, the binding and fusion steps are performed by a single viral entry protein. For example, influenza virus, HIV, Ebola virus, and Lassa virus, all use a single entry protein for binding and fusion with a host cell. For other viruses, multiple proteins are involved. For example, Nipah virus has separate binding and fusion proteins.

Viral entry proteins are a primary target of immune system responses against infection. Most vaccines elicit neutralizing antibodies to the viral entry protein. Therapeutic antibodies can also be used to impair the activity of viral entry proteins, with the potential to both protect against infection as well as therapeutically treat active infection. However, viral entry proteins are able to mutate and evolve, and mutations can allow these proteins to escape recognition by immune system responses and therapeutic antibodies. Evasion or susceptibility to antibodies can be examined using mutant viral entry proteins in antibody neutralization assays.

A virus's viral entry protein is also a key determinant of the species that the particular virus can infect, and adaptive evolution of these entry proteins has been retrospectively characterized in most molecularly documented examples of non-human viruses jumping into humans. For example, the influenza pandemics of 1918, 1957, and 1968 all involved mutations that turned viral entry proteins from avian viral strains to strains that could better infect humans. The severe acute respiratory syndrome (SARS) coronavirus outbreak in 2003 was associated with mutations in the virus's entry protein that enabled it to better bind human receptors. The MERS-CoV viral entry protein has mutations that increase binding to human cells. Recent evidence also suggests that during the 2014-2016 Ebola outbreak, this virus's entry protein acquired mutations that promoted infection of human cells. Comparing the growth of viral mutants in different cell types can serve to identify mutations that contribute to host adaptation.

Therefore, it would be incredibly useful to identify particular amino acids within a viral entry protein that are important for binding and fusion to host cells and/or antibody evasion. The entry proteins of a few viruses (e.g., influenza, HIV) are well-characterized, but surprisingly little is known about the entry proteins of many less-studied viruses in part because these proteins are challenging to study. They form large metastable oligomers that are often heavily modified with sugar molecules which render them difficult targets for biochemistry and structural biology.

WO2020/006494 describes an approach for performing deep mutational scanning of proteins. The systems and methods overcame biosafety and containment considerations by storing the library of genes encoding variant proteins in a non-infective state within holding cells. More particularly, the library is stored as barcoded non-replicative variants inside cells. A storage cell includes a non-self-inactivating viral vector integrated into the storage cell's genome, where the non-self-inactivating viral vector includes a single homozygous barcoded variant nucleotide sequence from a set of barcoded variant nucleotide sequences that encode viral protein variants forming a deep mutational scanning library of a viral protein. The integrated viral protein variants in cells are considered non-replicative because expression of viral genes (e.g., gag, pol, env, tat, rev) provided by the transfection of the cells with helper plasmids is needed for the production of virions. Virions produced from the transfection of cells storing a barcoded deep mutational scanning library of protein variants are non-replicative because the genome of each virion does not contain the full complement of viral genes needed for replication. Virion production can be induced by transfecting the storing cells with viral helper plasmids that encode the rest of the retroviral particle proteins. This results in expression in each cell of retroviral particles that are packaged with a barcoded gene encoding a given mutant viral entry protein and pseudotyped with that particular mutant viral entry protein. The ability to produce virions that package a barcoded gene encoding a mutant viral entry protein following transfection with helper plasmids is achieved, in part, through the use of a vector that is not self-inactivating, for example, by including a functional U3.

Following generation of virions from the storage cells, functional studies can be conducted to assess variant proteins.

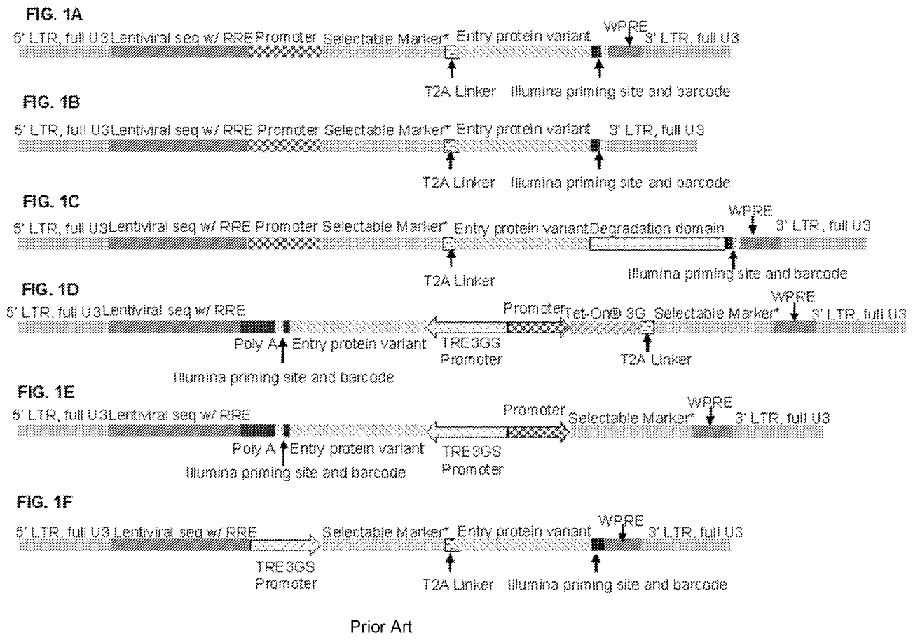

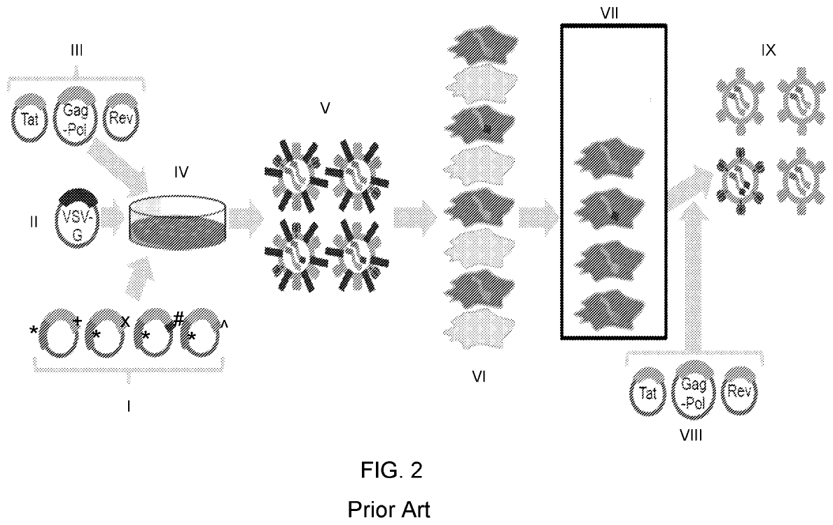

Schematics of the approach described in WO2020/006494 are depicted in FIGS. 1A-1D and 2. FIGS. 1A-1D depict exemplary lentiviral backbone constructs that can be used. Each genetic construct includes a codon variant that encodes a viral entry protein. Exemplary methods to create a library of codon variants expressing viral entry proteins are described below.

While the libraries described in WO2020/006494 provide some important advances, opportunities to further improve the libraries remain.

The current disclosure provides modifications and new uses of the libraries described in WO2020/006494. The modifications further improve the WO2020/006494 libraries.

One modification utilizes an inducible promoter for the variant viral proteins to better control viral protein expression levels. Particular embodiments utilize a reverse tetracycline transactivator (rtTA). rtTA functions differently than the original tetracycline-controlled transactivator (tTA) in that tTA turns off expression when tetracycline is introduced while rtTA turns on expression when tetracycline is introduced.

Particular embodiments utilize viral backbones that operably connect the variant protein of study to a particular inducible promoter and operably connect a reporter and resistance gene to a different promoter. For example, the variant protein can be operably connected to the inducible rtTA promoter, while the reporter and resistance gene, for example, ZsGreen linked to puromycin resistance (PuR) via a T2A linker, are operably connected to a constitutive CMV promoter.

Particular embodiments utilize the TetOn-3G system (Takara). Other inducible promoters may also be used, as described elsewhere herein.

Another modification utilizes linear amplification of viral proteins, so that there are more barcodes available for sequencing.

Particular embodiments utilize amplification (e.g., T7 amplification) of barcode sequences after selections with the virus libraries. In these embodiments, barcodes are amplified before sequencing either in the cells before isolating the viral genomes or after isolating the viral genomes from the cells with minipreps.

Another modification creates an environment permissive to variant protein expression and/or viral/target cell interaction by: (i) providing or enhancing pro-viral factors in producer cells; (ii) removing or otherwise inhibiting anti-viral factors from producer cells; (iii) providing or enhancing pro-viral factors in target cells; and/or (iv) removing or otherwise inhibiting anti-viral factors from target cells. By altering these factors either with drugs or genetically, the viral titer for experiment can be increased, making the experiments easier to perform. Higher effective titer makes experiments easier to perform because it allows the use of smaller volumes to get higher coverage of the variant libraries when doing selections, which allows the use of less antibody or serum for neutralization selections. This can be important for precious samples that should not be depleted quickly.

In certain instances, it may also be helpful to create an anti-viral environment. In these embodiments, an environment less permissive to variant protein expression and/or viral/target cell interaction can be created by: (i) providing or enhancing anti-viral factors in producer cells; (ii) removing or otherwise inhibiting pro-viral factors from producer cells; (iii) providing or enhancing anti-viral factors in target cells; and/or (iv) removing or otherwise inhibiting pro-viral factors from target cells.

Another modification includes using spike in controls for reference levels based on proteins that are not subject to pre-existing immunity in humans.

Particular embodiments utilize pseudotyped standards (e.g., VSV G pseudotyped standards) as controls, wherein the pseudotyped standards are produced in the same way as the virus library in parallel. These standards can encode a reporter protein (e.g., mCherry) rather than a variant protein in their genomes, and have a small pool of possible barcodes (e.g., 8). These standard genomes can be integrated in, for example, 293T-rtTA-mCherry cells, as the library and in parallel, and then rescued by transfecting helper plasmids and a plasmid expressing VSV G to acquire a VSV G pseudotyped standards pool produced in the same manner as the libraries. This pool can then be spiked in during selections on the libraries to be around 0.5-2% of the total virus pool.

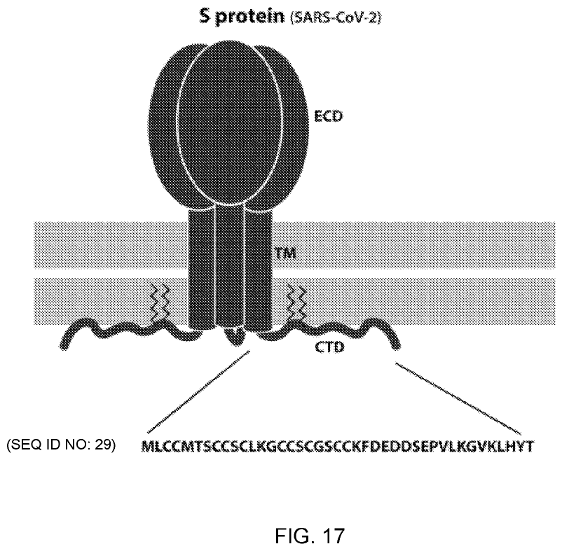

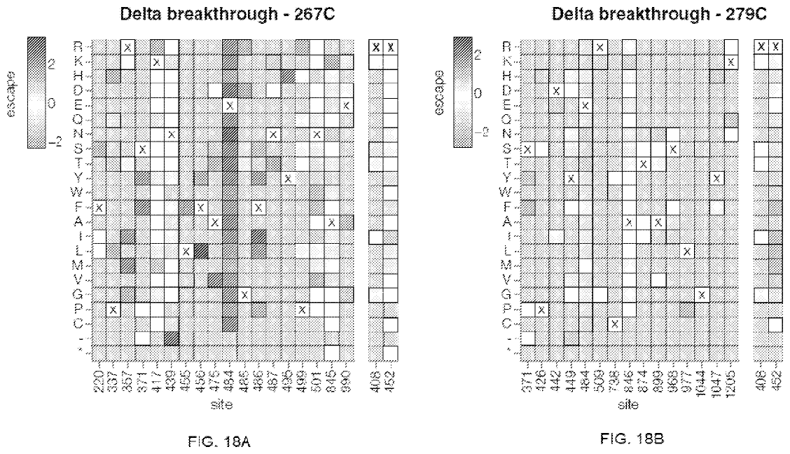

Another modification utilizes the membrane-bound surface expression of viral proteins (FIG. 17). Viral entry proteins are inherently transmembrane proteins, meaning they have a part of the protein that traverses the cell membrane and fixes them in the cell membrane. Thus, expressing these proteins from integrated lentivirus genomes results in cells that “display” the variant proteins on cell the surface.

In additional embodiments, the cytoplasmic tail of viral entry proteins can be deleted. As just one example, for the SARS-CoV-2 spike protein, the last 21 amino acids of the protein can be deleted. Similar modifications may be made for other viral entry proteins such as those for Nipah and RSV. This modification can be employed because cytoplasmic tails often have various retention signals that traffic them in intracellular compartments and limit the amount of protein that eventually reaches the cell membrane. Thus, removing cytoplasmic tails can increase protein surface expression.

The modifications described above and elsewhere herein can be practiced individually or in any combination to achieve powerful DMS libraries.

Aspects of these improvements are described in the following discussion regarding the use of disclosed libraries in relation to the SARS-CoV-2 spike protein and HIV Env protein.

The spike protein is the key target of neutralizing antibodies against SARS-CoV-2. Unfortunately, spike has undergone rapid evolution which has eroded the potency of serum neutralization and escaped many monoclonal antibodies (Cao et al., 2022, bioRxiv 2022.09.15.507787; Liu et al., 2022, Nature 602, 676-681; Wang et al., 2022, Cell Host Microbe. doi.org/10.1016/j.chom.2022.09.002; Wang et al., 2022, Nature 608, 603-608). Deep mutational scanning experiments can prospectively measure the effects of large numbers of mutations even before they emerge in viral variants, and therefore have been a valuable tool for rapidly interpreting how newly observed mutations in the spike affect antibody binding and protein folding or function (Cao et al., 2022, bioRxiv 2022.09.15.507787; Starr et al., 2021, Nature 597, 97-102; Starr et al., 2021, Science 371, 850-854). The high-throughput nature of deep mutational scanning experiments has also enabled the generation of huge datasets that can inform computational methods for predicting the antigenic properties of possible future viral variants (Cao et al., 2022, bioRxiv 2022.09.15.507787; Greaney et al., 2022, Virus Evol. 8, veac021).

However, prior deep mutational scanning of the SARS-CoV-2 spike has been limited to either solely focusing on the receptor-binding domain (RBD) (Cao et al., 2022, bioRxiv 2022.09.15.507787; Greaney et al., 2021, Nat. Commun. 12, 4196; Starr et al., 2020, Cell 182, 1295-1310.e20), other subdomains (Ouyang et al., 2022, bioRxiv 2022.06.20.496903; Tan et al., 2022, bioRxiv 2022.09.24.509341) or just a small number of mutations across spike (Javanmardi et al., 2021, Mol. Cell 81, 5099-5111.e8). Furthermore, all previous spike deep mutational scanning experiments have been based on cell-surface display using either yeast (Starr et al., 2022, doi.org/10.1101/2022.09.20.508745; Starr et al., 2020, Cell 182, 1295-1310.e20) or mammalian cells (Javanmardi et al., 2021, Mol. Cell 81, 5099-5111.e8; Ouyang et al., 2022, bioRxiv 2022.06.20.496903; Tan et al., 2022, bioRxiv 2022.09.24.509341), and therefore are limited to measuring antibody binding rather than neutralization, despite the fact that neutralization is thought to be a more relevant correlate of protection (Feng et al., 2021, Nat. Med. 27, 2032-2040; Gilbert et al., 2022, Science 375, 43-50).

Efforts to create an HIV vaccine have been stymied in part by the rapid and continuing diversification of the virus's envelope (Env) protein. While progress has been made in characterizing individual broadly neutralizing antibodies, individual antibodies do not always recapitulate the neutralizing activity of the serum of the individuals from whom they were isolated. Further, mapping the specificity of polyclonal neutralizing serum is more difficult than characterizing individual monoclonal antibodies. Current approaches characterize binding rather than neutralizing specificity, and it has been determined that many serum antibodies bind non-neutralizing epitopes Fingerprinting approaches may define neutralizing epitopes, but do not provide mutation-level specificity and require making measurements for large virus panels. Precisely mapping neutralizing specificities and escape mutations is especially challenging for antibodies that target the CD4-binding site. Such antibodies recognize conserved Env residues while typically avoiding steric clashes with glycans rather than depending on them for neutralization, unlike antibodies targeting other epitopes such as the V1/V2 loops or V3 loop. As a result, CD4-binding-site-targeting antibodies can have near pan-HIV neutralization breadth and high potency despite sequence and glycan heterogeneity across strains of HIV and are therefore promising candidates for treatment and prophylaxis strategies, but the higher conservation of their epitopes can also make it more difficult to map escape mutations for such antibodies.

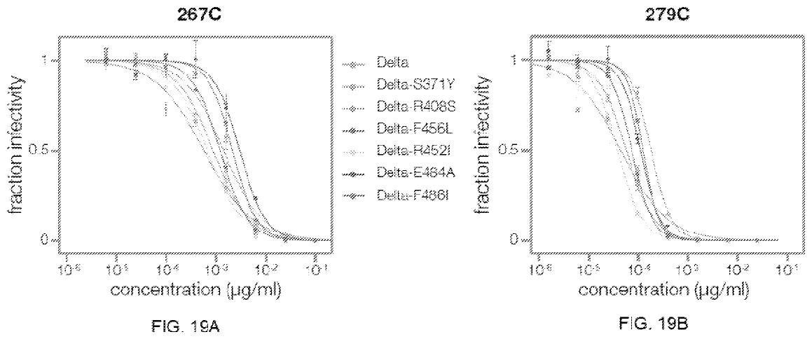

Here, a new deep mutational scanning platform is described that directly measures how mutations affect cellular infection and antibody neutralization in the context of the full SARS-CoV-2 spike pseudotyped on non-replicative lentiviral particles. A similar platform can measure how mutations affect the neutralization of Env by human anti-HIV sera that target the CD4 binding site. The system can also measure combinations of mutations, enabling quantitative deconvolution of how mutations mediate escape at distinct antibody epitopes. A key innovation behind the platform is a two-step pseudovirus generation protocol that enables the creation of large pseudovirus libraries with a link between the lentiviral genotype and the particular spike protein variant on the pseudovirus's surface or the lentiviral genotype and the HIV Env on the pseudovirus's surface. This new platform can be used to create large genotype-phenotype linked pseudovirus libraries and map how mutations to spike affect both cellular infection and neutralization by antibodies targeting diverse regions of the spike, including the RBD, N-terminal domain (NTD), and S2 domain. A similar platform can shed light on the specificity of human serum that can broadly neutralize many HIV strains. The methods may be used to evaluate and compare the neutralizing specificities of anti-HIV sera elicited by different vaccine regimens. The platform enables the creation of large libraries of single-round replicative lentiviruses with a genotype-phenotype link between barcodes in the lentivirus genomes and the mutant HIV Env entry proteins on the surfaces of virions (FIG. 21A).

Producing pseudoviruses with genotype-phenotype link. To characterize thousands of mutations in spike glycoprotein, a lentiviral pseudotyping platform was established that maintains a genotype-phenotype link between the lentiviral genome and the spike variant on the virion's surface. Traditional lentiviral spike-pseudotyping involves transfection of a backbone that carries a reporter gene flanked by the lentiviral long terminal repeats (LTRs), helper plasmids that code for structural and nonstructural genes required for the lentiviral life cycle, and an expression plasmid that codes for the spike variant of interest (Crawford et al., 2020, Viruses 12, 513; Cronin et al., 2005, Curr. Gene Ther. 5, 387-398; Naldini et al., 1996, Science 272, 263-267). When these components are transfected into producer cells, virions are formed that carry lentiviral genomes and display spikes on their surface. However, because genome incorporation into a virion does not depend on the expressed spike, there is no link between the virion's genotype and the phenotype of the spike on its surface. The absence of a genotype-phenotype link is not problematic when only a single spike variant is used for transfection-however, it precludes deep mutational scanning studies that involve studying thousands of variants in a single pooled experiment.

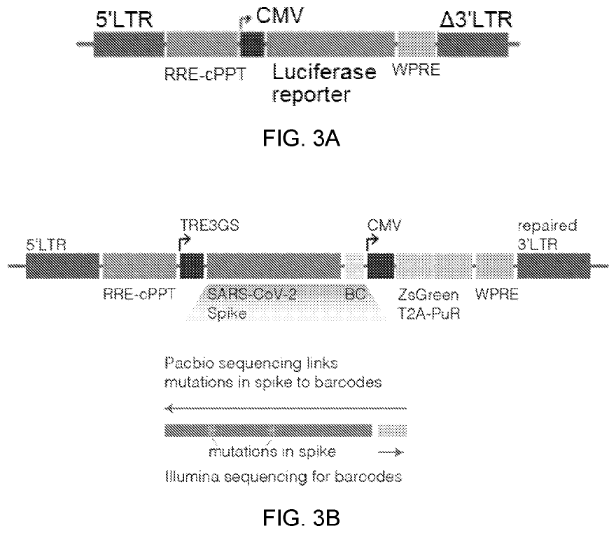

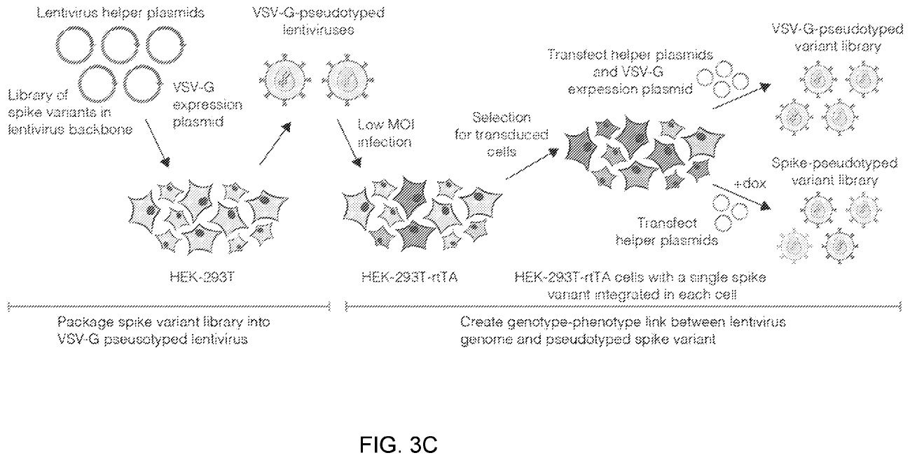

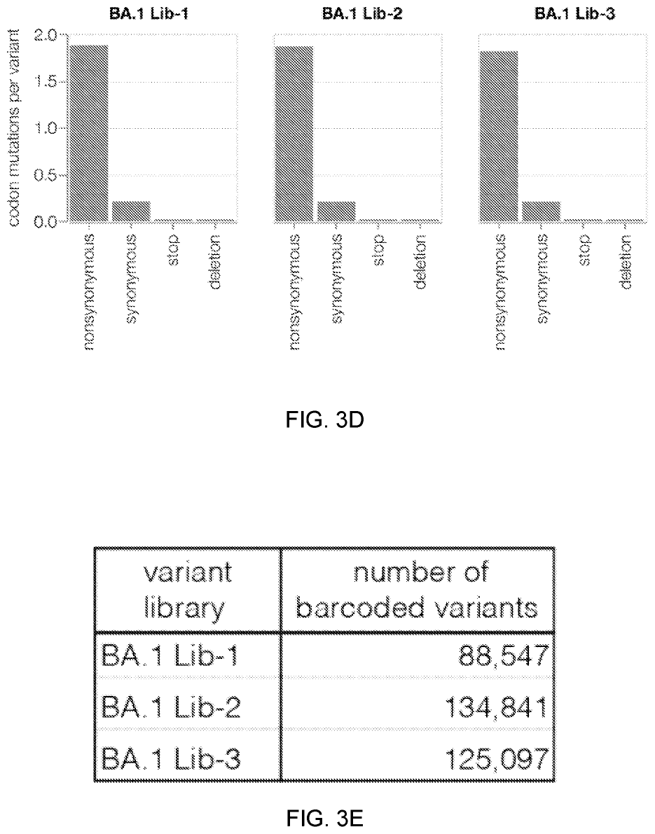

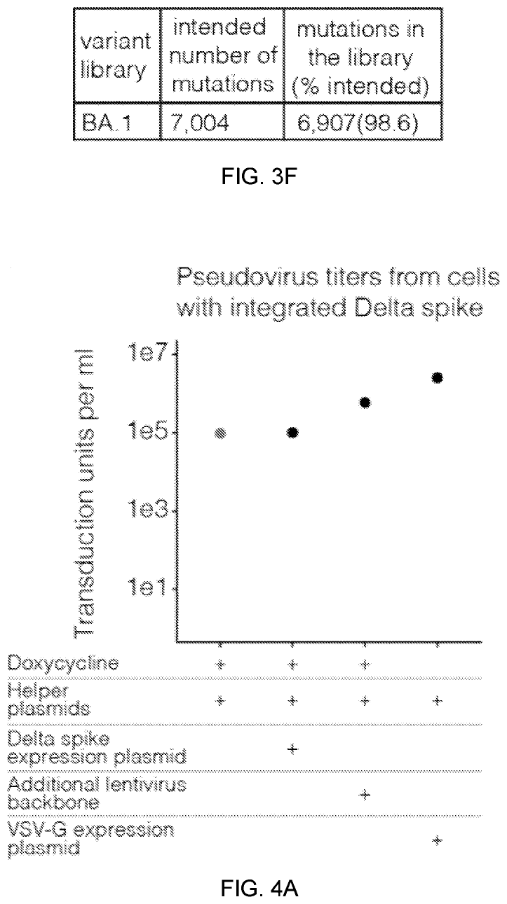

To create a lentiviral genotype-phenotype link, a lentivirus backbone was generated with the following key elements (FIG. 3B) and (FIG. 21A): (1) the ability of the lentivirus to transcribe its full genome after integration by repairing the 3′ LTR deletion present in traditional lentivirus vectors (Zufferey et al., 1998, J. Virol. 72, 9873-9880), (2) a spike or HIV Env mutant was placed in the lentivirus backbone under an inducible promoter, (3) a second constitutive promoter was added to drive both a fluorescent reporter (ZsGreen) and a puromycin resistance gene.

Next, a multi-step protocol was developed that creates a genotype-phenotype link by ensuring that each producer cell only expresses a single variant of spike (FIG. 3C) or Env mutant (FIG. 21B) respectively. In the first step of this protocol, cells were transfected with the spike-encoding backbone or barcoded Env mutants, a VSV G expression plasmid, and the necessary helper plasmids. This produces non-genotype-phenotype-linked VSV G pseudotyped lentiviruses that were used to infect target cells at low multiplicity of infection so that most infected cells receive no more than one lentiviral genome.

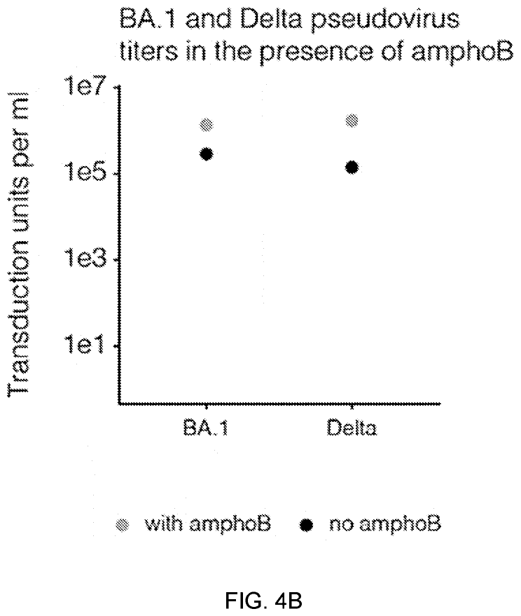

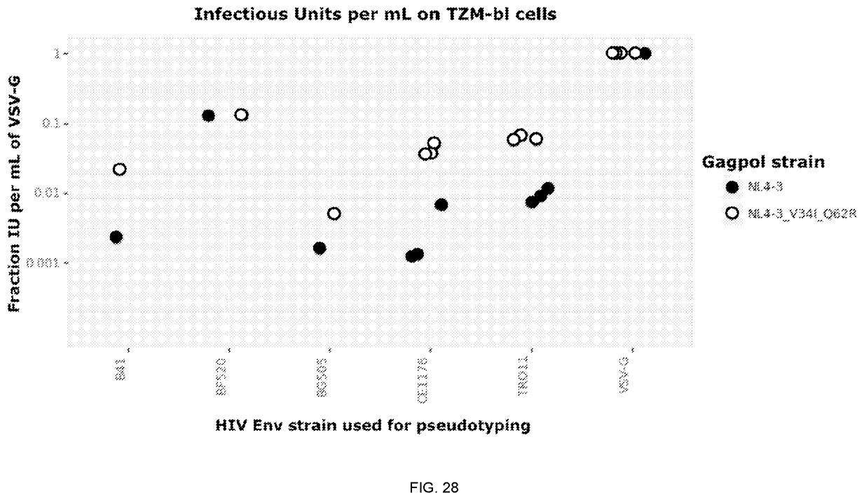

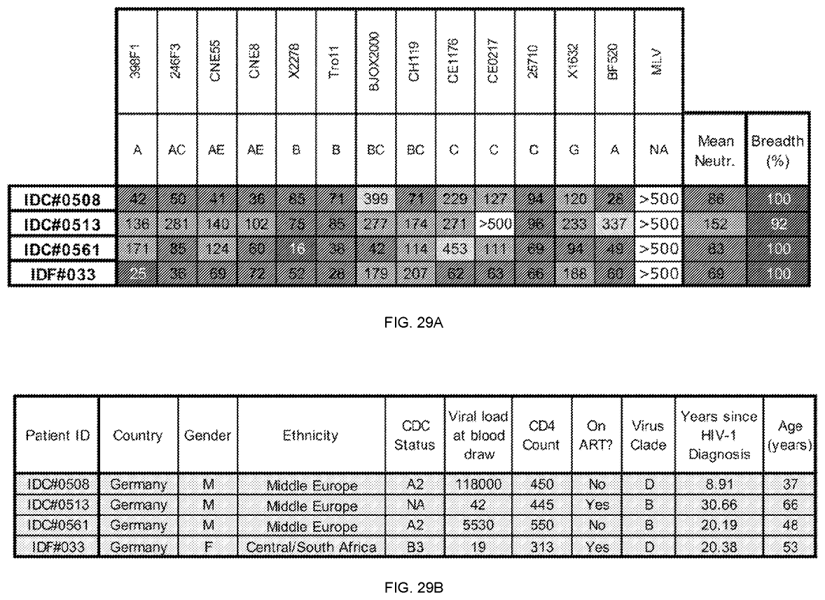

Next, cells were selected for integrated lentiviral genomes using puromycin, which yields a population of cells where each cell stores only a single spike variant or Env mutant. The spike or Env mutants are under an inducible promoter, which is only activated by the addition of doxycycline. To produce virions, spike expression or Env expression was induced with doxycycline and transfected the helper plasmids necessary to produce lentiviruses. This approach can be used to generate genotype-phenotype linked spike-pseudotyped viruses with titers >10⁵ transduction units per ml (FIG. 4A). Viral titers can further be increased by 5-10 fold by infecting cells in the presence of a putative IFITM3 inhibitor amphotericin B (Lin et al., 2013, Cell Rep. 5, 895-908), as has been reported previously (Peacock et al., 2021, Nat. Microbiol. 6, 899-909; Zhao et al., 2020, J. Virol. 94, e00562-20; Zheng et al., 2020, Microbes Infect. 9, 1567-1579) (FIG. 4B). The titers for genotype-phenotype-linked Env expressing viruses were1.5-35 million infection units per mL (FIG. 28) (Freed et al., J. Virol. 1996; 70: 341-351; Tedbury et al., PLoS Pathog. 2013; 9 (e1003739)).

Design of mutations in SARS-CoV-2 spike deep mutational scanning library. Rather than creating deep mutational scanning libraries containing all possible amino-acid mutants of spike only mutations that seem likely to arise during natural evolution and yield a functional spike protein. There were two rationales for designing the disclosed libraries in this way: (1) it reduces the total number of mutations that need to be included in the library, and (2) it increases the probability that variants with multiple mutations will remain functional by reducing the fraction of highly deleterious mutations.

Specifically, only mutations that have been observed in spike sequences deposited on the GISAID database were included (Khare et al., 2021, GISAID's Role in Pandemic Response. China CDC Wkly. 3, 1049-1051), reasoning that these mutations would represent mostly functional spike proteins. Mutations were introduced at a higher frequency when they have emerged in spike independently many times according to the pre-built SARS-CoV-2 phylogenies from UShER (Turakhia et al., 2021, Nat. Genet. 53, 809-816). Finally, every possible amino acid change was included at sites in spike that are evolving under positive selection (Maher et al., 2022, Sci. Transl. Med. 14, eabk3445). Deletions were also included at sites where such mutations are observed frequently in natural SARS-CoV-2 evolution. In total, the disclosed library design targeted 7,004 mutations in the BA.1 spike and 6,852 mutations in the Delta spike.

To introduce these mutations in the spike gene a PCR-based mutagenesis method with a primer pool containing the desired mutations was used (Bloom, 2014, Phylogenetic Fit. Mol. Biol. Evol. 31, 1956-1978). Importantly, this method introduces multiple mutations in each spike variant: 2 to 3 codon mutations were targeted per variant, ensuring the effects of most mutations are measured in multiple genetic backgrounds.

The PCR-based mutagenesis spike genes were then barcoded with 16 random nucleotides placed downstream of the spike-coding sequence (FIG. 3B) and cloned into the lentivirus backbone. As described below, after integration of the libraries into cells, these barcodes can be linked to the full set of mutations in each spike variant to facilitate downstream sequencing (Hiatt et al., 2010, Nat. Methods 7, 119-122; Matreyek et al., 2018, Nat. Genet. 50, 874-882).

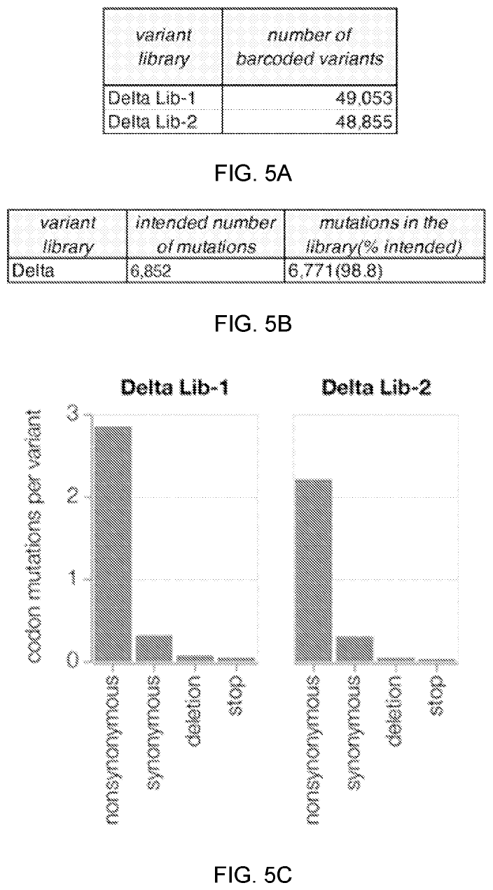

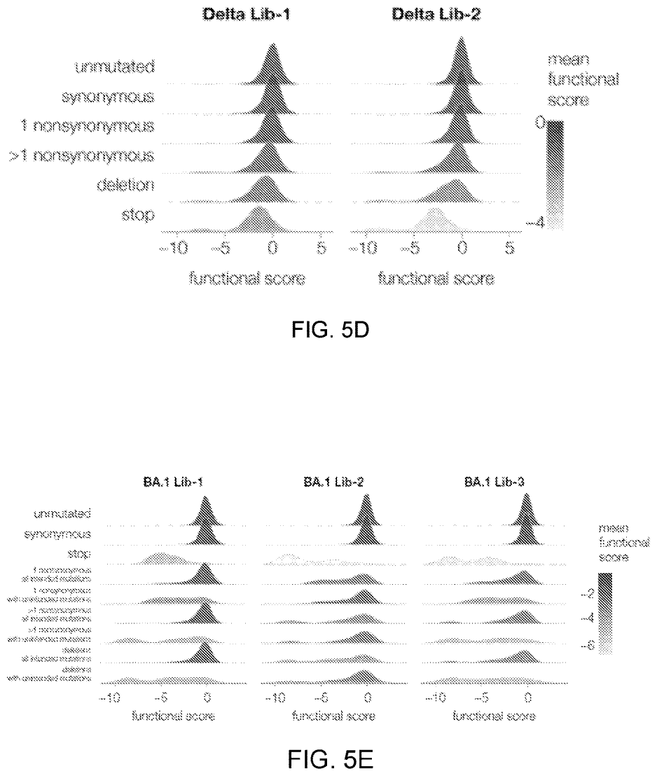

Production of pseudotyped BA.1 and Delta spike deep mutational scanning libraries. The genotype-phenotype linked pseudovirus production strategy in FIG. 3C was used to make BA.1 and Delta deep mutational scanning libraries. Three independent BA.1 libraries were created, each containing 100,000 barcoded variants, and two independent Delta libraries each containing 50,000 barcoded variants (FIGS. 3F, 5A; a “barcoded variant” is a spike with a unique nucleotide barcode and some random mutation set; different barcoded variants usually but not always contain different mutations). After integrating the libraries into cells at low multiplicity of infection, VSV G pseudotyped lentivirus was generated from these cells by co-transfecting a plasmid expressing VSV G alongside the other lentiviral helper plasmids (FIG. 3C, top right). The use of VSV G pseudotyped virus ensures that infectious lentiviral virions were generated from all integrated backbones independently of the functionality of the spike mutant they encode. This VSV G pseudotyped lentivirus was then infected into a new round of cells, and long-read PacBio sequencing was performed to link the barcodes to the full set of spike mutations for each variant. PacBio barcode-mutation linking was performed after integration into cells because recombination of the pseudodiploid lentiviral genome during integration (Jetzt et al., 2000, J. Virol. 74, 1234-1240; Schlub et al., 2010, PLOS Comput. Biol. 6) means the barcode-mutation pairings may be different in the integrated cells than the original lentiviral backbone plasmids (Hill et al., 2018, Nat. Methods 15, 271-274). Importantly, linking barcodes to spike variants allows for the use of short-read Illumina sequencing of the barcode to obtain the full spike genotype in all subsequent experiments.

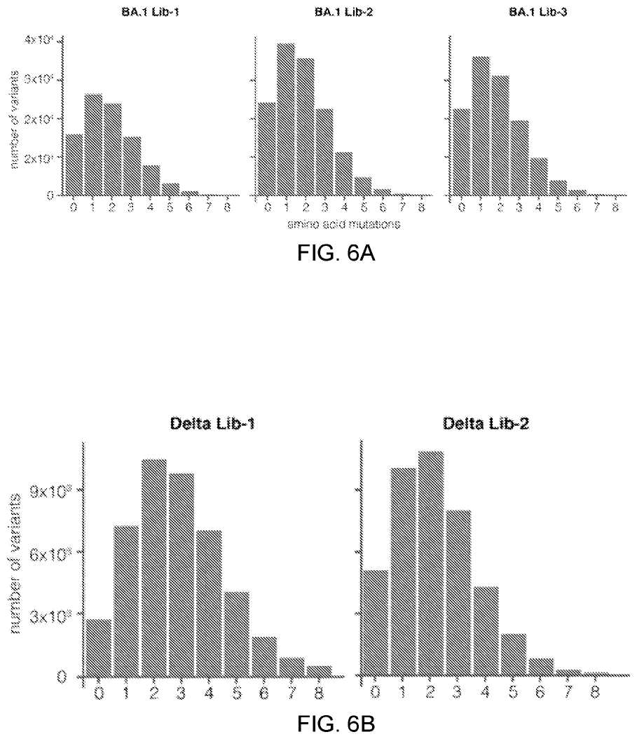

Overall, the sequencing revealed the successful introduction of 99% of the targeted mutations in the BA.1 and Delta spike libraries (FIGS. 3F, 5B). The barcoded variants in the BA.1 libraries had on average 2 codon mutations per spike, while the variants in the Delta libraries had 3 codon mutations per spike (FIGS. 3D, 5C). The number of mutations per variant is roughly Poisson distributed, so some variants had zero or one mutation, while others had many more (FIG. 6A).

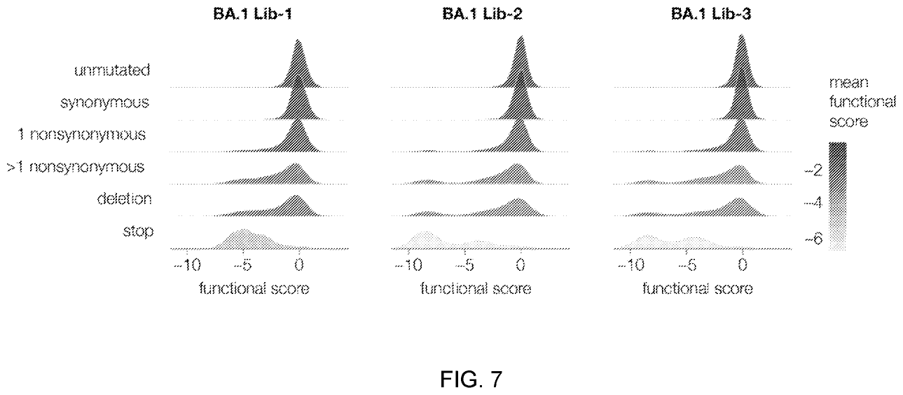

The actual spike-pseudotyped deep mutational scanning libraries were then generated from the variants stored as a single copy in the cells (FIG. 3C, lower right). A functional score was calculated for each variant based on its relative frequency in the spike versus VSV G pseudotyped libraries. Positive functional scores indicate spike variants mediate pseudovirus infection better than the parental spike, whereas negative functional scores indicate worse pseudovirus infection. As expected, spike variants with premature stop codons had highly negative functional scores, while unmutated and synonymously mutated spike variants had functional scores close to zero (FIGS. 7, 5D). Some variants with nonsynonymous mutations had functional scores close to zero, while others had more negative scores, reflecting the fact that some but not all nonsynonymous mutations are deleterious (FIGS. 7, 5D; recall that the disclosed library design protocol preferentially introduced nonsynonymous mutations expected to yield functional spikes). Variants with multiple nonsynonymous mutations tended to have lower functional scores than variants with just one nonsynonymous mutation (FIGS. 7, 5D), reflecting the cost of accumulating multiple often mildly deleterious mutations.

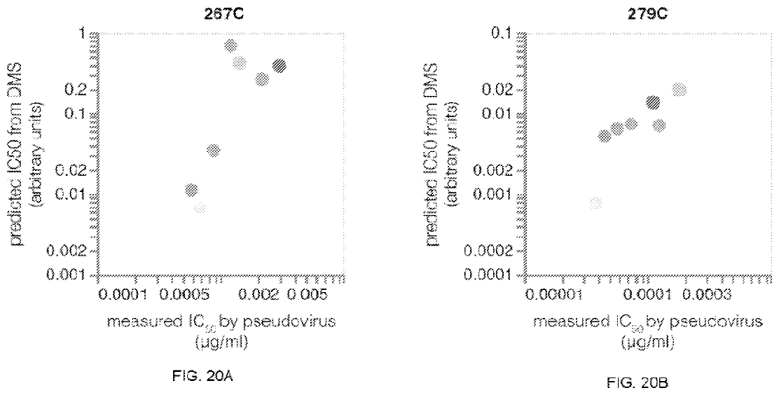

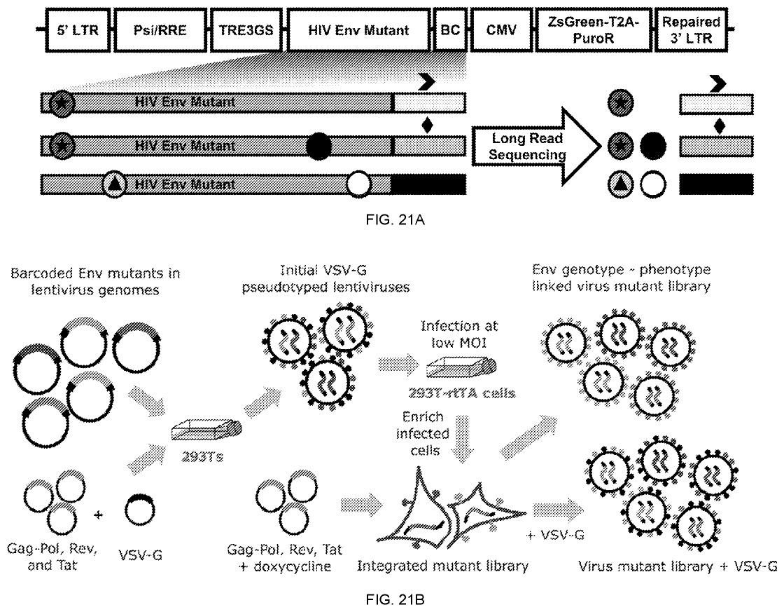

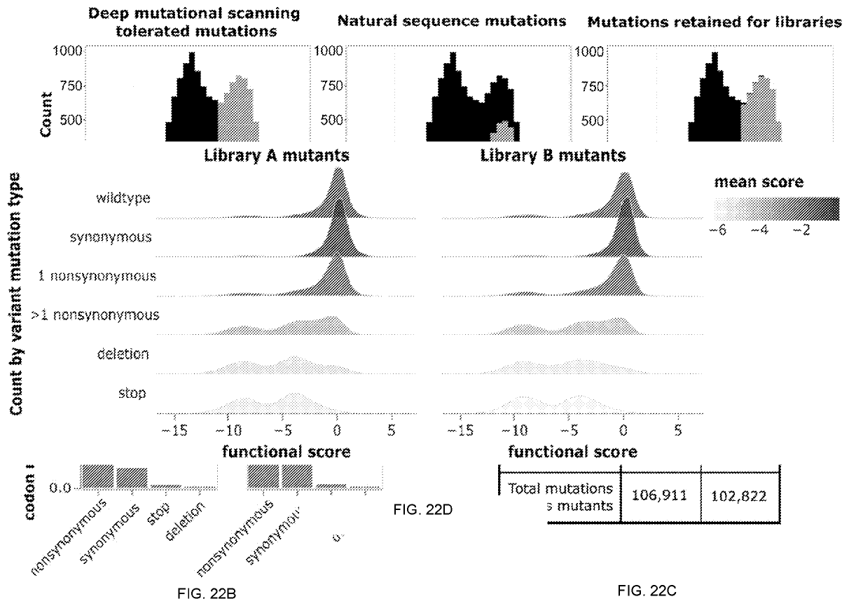

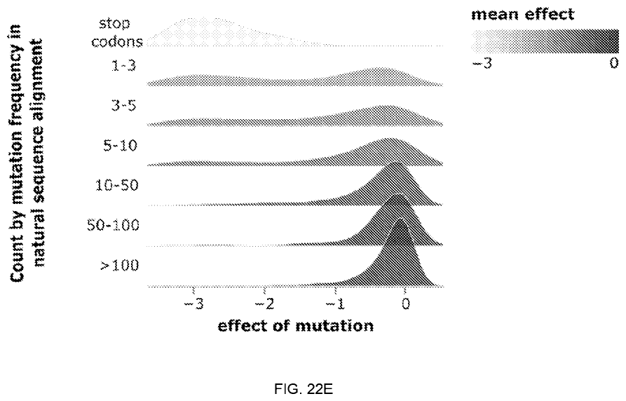

Design of Env mutant deep mutational scanning library. While any Env may be used, in some aspects Env from the transmitter/founder virus BF520.W14M.C2 (BF520). Transmitted/founder viruses are particularly relevant for antibody/neutralization studies as they are more challenging to neutralize with antibodies. Prior studies of BF520 Env generated using full-length replicative HIV virions in a system that could only measure the average effect of mutations across different genetic backgrounds were reviewed to identify well-tolerated mutations (FIG. 22A, left panel). An alignment group of M HIV-1 sequences was used to identify any mutations relative to BF520 present more than once in natural sequences (FIG. 22A, middle panel). The library design included 7110 amino acid mutations in the BF520 ectodomain that were either tolerated in the prior deep mutational scanning or present multiple times in the natural sequence alignment (FIG. 22A, right panel).

Two independent Env mutant libraries were generated. PacBio sequencing showed that each library had 2.5 nonsynonymous mutations per Env mutant, which are linked via the barcode and can therefore be evaluated in combination (FIG. 21A). There was a low frequency of synonymous mutations, stopcodons, and in-frame deletions (FIG. 22B). Overall, 84% of the mutations were among the 7,110 mutations targeted by the library design. Each library contains 40,000 barcoded mutations, and together the two libraries sampled 97% of the targeted mutations (FIG. 22C).

To evaluate how mutations affected the ability of Env to mediate viral infection in cell culture, libraries pseudotyped with just the Env mutants or Env mutants with vesicular stomatitis virus G protein (VSV G) were generated (FIG. 21B). The libraries were then used to infect TZM-bl cells which express Env's primary receptor (CD4) and co-receptors (CCR5 and CXCR4). All virons with functional Envs are expected to infect cells when VDV-G is present, but only virons with functional Envs will infect cells in the absence of VSV G. Each barcoded Env variant was assigned a functional score calculated as the log of the ratio of the frequency of the variant (relative to unmutated B520 Env) in the Env versus VSV G mediated infections. Negative functional scores indicate an Env mutant is worse at infecting cells than unmutated BF520 Env, whereas positive functional sores indicate it is better at infecting cells.

Env mutants with only synonymous mutations have “wild-type-like” functional scores of near zero whereas mutants with stop codons generally have highly negative functional scores (FIG. 22D). Most mutants in the libraries with only one nonsynonymous mutation have functional scores close to zero, suggesting that the library design largely incorporated functionally tolerated mutations as intended. Env mutants with multiple nonsynonymous mutations more often have substantially negative functional scores, as expected from the accumulation of multiple sometimes deleterious mutations (FIG. 22D). Mutations found more often among natural sequences tend to have more favorable effects in these experiments than mutations rarely found among natural sequences (FIG. 22E) suggesting that mutations that are favorable for viral entry in these experiments are generally also favorable during natural HIV evolution.

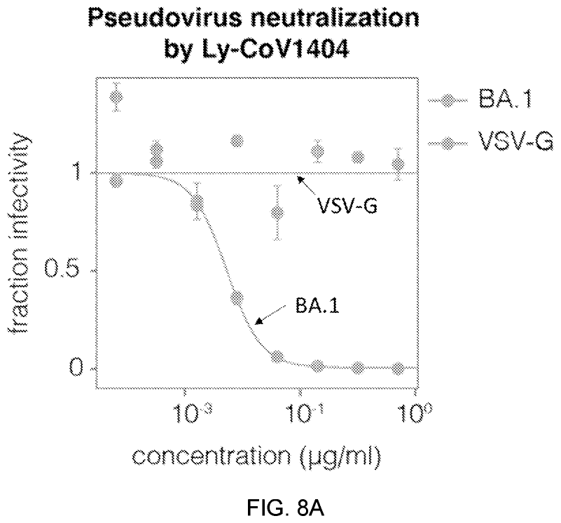

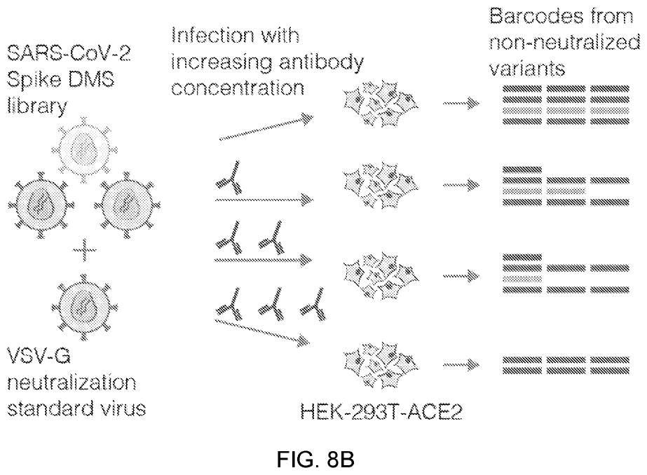

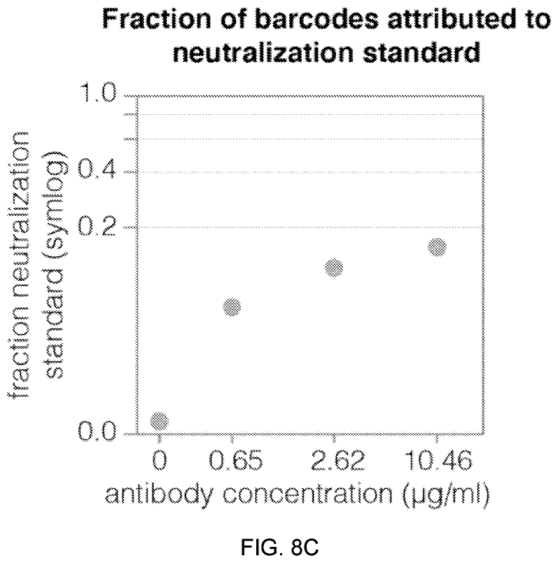

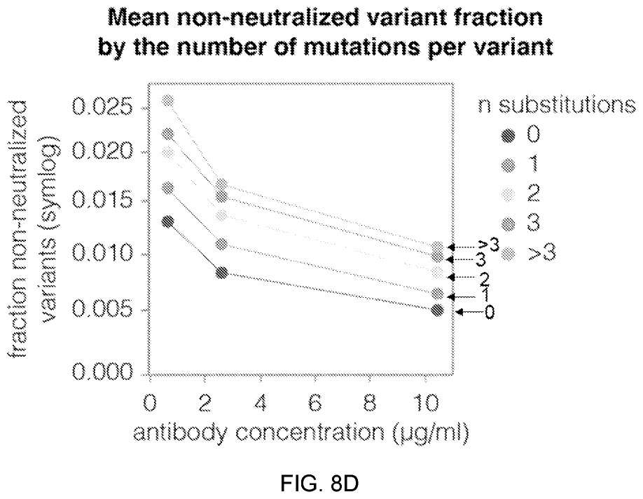

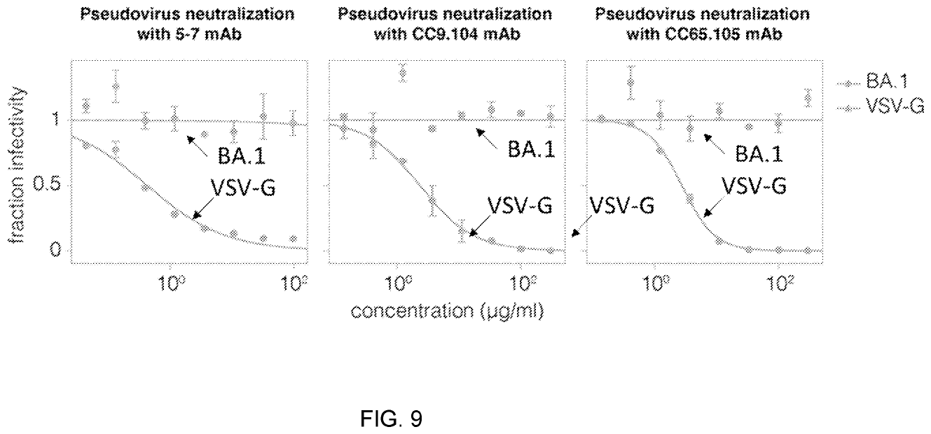

Use of an absolute standard to measure viral neutralization by deep sequencing. Traditional neutralization assays measure the infectivity of a single virus variant at multiple antibody concentrations. Deep sequencing can measure the relative infectivities of many viral variants in pooled infections in the presence of an antibody. However, to convert the relative infectivities measured by deep sequencing into actual neutralization values, it is necessary to have an absolute standard that does not vary in its infectivity as a function of antibody concentration (FIG. 8A). To enable such measurements in the disclosed experiments, a barcoded VSV G pseudotyped virus into the disclosed libraries was added. Importantly, this VSV G pseudotyped virus is not neutralized by any of the spike binding antibodies (FIGS. 8A, 9), and so the counts for VSV G barcodes provide an absolute neutralization standard. To calculate the non-neutralized fraction for each viral variant, one can compute the change in its barcode frequencies relative to the VSV G standard (FIG. 8B). Similar procedures may be used for Env binding antibodies.

To validate this approach, the VSV G absolute standard was added at 1% of the disclosed BA.1 library titers and the virus library was incubated with increasing concentrations of the Ly-Cov1404 antibody, as schematized in FIG. 8B. The library was then infected into ACE2-expressing target cells overnight, viral genomes were recovered, and the abundance of each viral barcode was quantified using deep sequencing. As expected, the fraction of VSV G standard reads increased with antibody concentration because fewer spike variants could still infect in the presence of antibody (FIG. 8C). The non-neutralized fraction for each viral variant in the disclosed libraries was then quantified after selection at different concentrations of the antibody. As expected, increasing antibody concentrations led to decreased non-neutralized fraction averaged over variants (FIG. 8D). Notably, variants with a greater number of substitutions had higher non-neutralized fractions, as expected if some substitutions escaped the antibody.

Mapping antibody escape using full spike deep mutational scanning system. To demonstrate that pseudovirus-based deep mutational scanning can map escape from neutralizing antibodies targeting any region of spike, a set of BA. 1-neutralizing antibodies that bind distinct regions of spike was chosen: RBD-binding Ly-CoV1404, NTD-binding 5-7, and S2-binding CC67.105 (Cerutti et al., 2021, Cell Rep. 37, 109928; Westendorf et al., 2022, bioRxiv 2021.04.30.442182; Zhou et al., 2022, bioRxiv 2022.03.04.479488). Note that Ly-CoV1404, also known as bebtelovimab, is one of the few clinically approved antibodies that retains potency against BA.1, BA.2, and other major Omicron lineages (Wang et al., 2022, Nature 608, 603-608; Westendorf et al., 2022, bioRxiv 2021.04.30.442182).

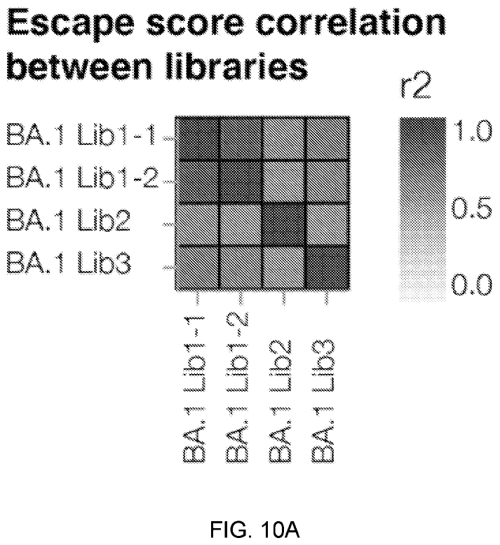

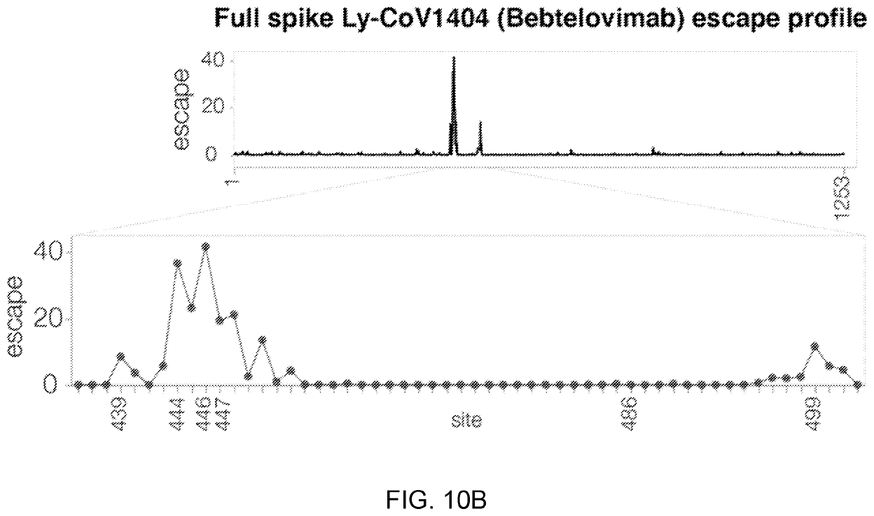

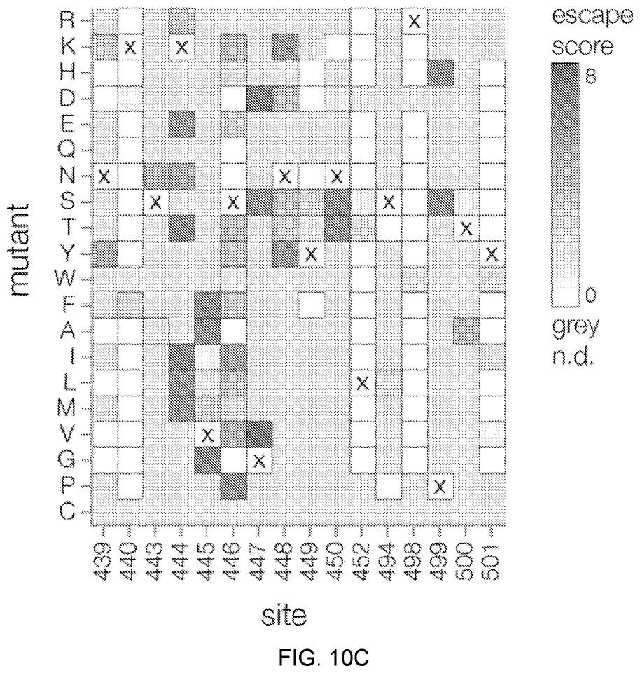

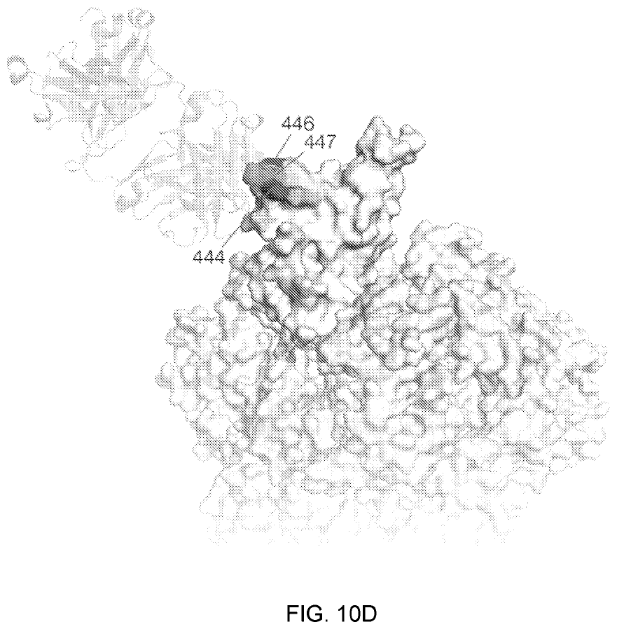

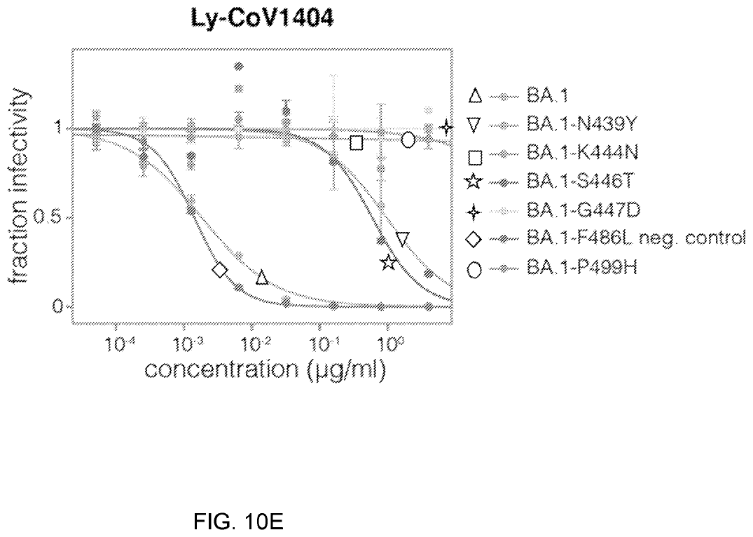

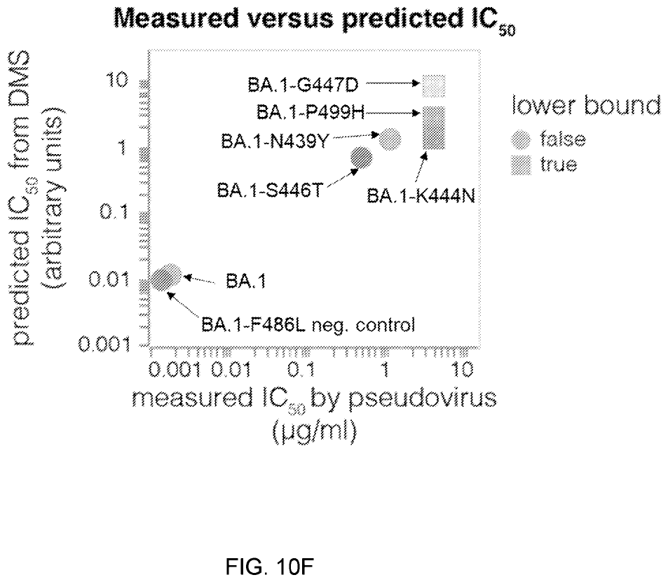

Escape from Ly-CoV1404 was mapped by applying the approach outlined in FIG. 8B to the disclosed three independent BA.1 libraries and performing a technical replicate for one library. A biophysical model was used to decompose the measurements for the spike variants in the disclosed libraries (some of which are multiply mutated) into escape scores for individual mutations (Yu et al., 2022, bioRxiv 2022.09.17.508366). These mutation escape scores correlated well among both the technical and biological replicates (FIG. 10A). As expected, the key Ly-CoV1404 escape sites were in the antibody's previously described epitope in the RBD (Westendorf et al., 2022, bioRxiv 2021.04.30.442182), which spans sites 439-452 and 498-501 (FIGS. 10B-10D). However, the disclosed deep mutational scanning emphasizes that only some mutations at these sites escape Ly-CoV1404 neutralization. For instance, many amino acid mutations at site 446 strongly escape Ly-CoV1414, but mutating this site from G (the identity in Wuhan-Hu-1) to S (the identity in BA.1 and BA.2.75) does not have a large effect. This observation emphasizes the somewhat serendipitous nature of the preserved potency of Ly-CoV1404. However, this antibody may soon be escaped because sub-variants of BA.5 and BA.2.75 with mutations in the key escape site of K444 are increasingly being detected (Chen et al., 2022, Bioinformatics 38, 1735-1737).

To validate the Ly-CoV1404 deep mutational scanning, a set of mutations was cloned in the BA.1 spike with a range of effects in the deep mutational scanning data, and standard pseudovirus neutralization assays were performed (FIG. 10E). All of the tested mutations exhibited neutralization phenotypes consistent with those measured in the deep mutational scanning. Furthermore, the neutralization assay IC₅₀ values correlated well with those predicted by the disclosed biophysical model (Yu et al., 2022, bioRxiv 2022.09.17.508366) parameterized by the deep mutational scanning data (FIG. 10F).

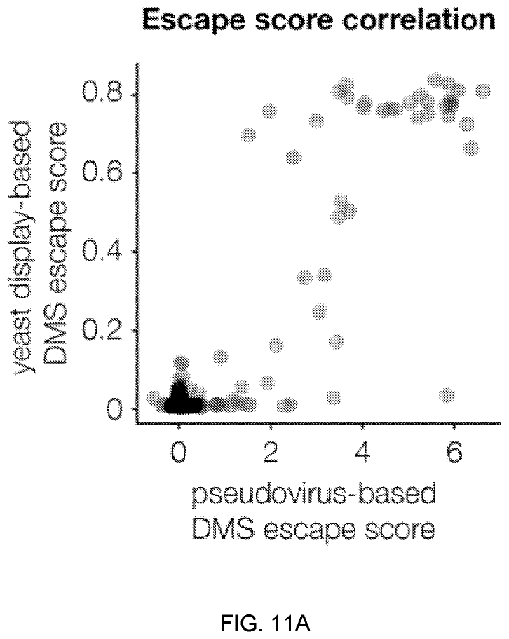

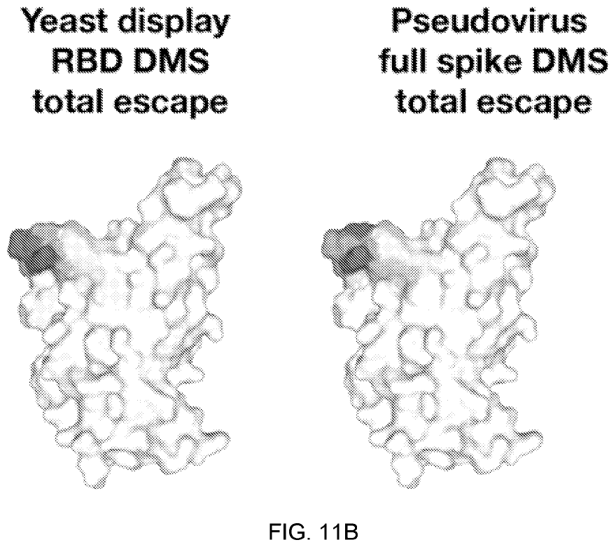

The full spike Ly-CoV1404 deep mutational scanning measurements were also compared to results from previously described yeast-display system for deep mutational scanning of only the RBD (Starr et al., 2022, doi.org/10.1101/2022.09.20.508745; Starr et al., 2020, Cell 182, 1295-1310.e20). The escape scores between the two experimental approaches correlated well (FIG. 11A) and both methods identified the same epitope (FIG. 11B).

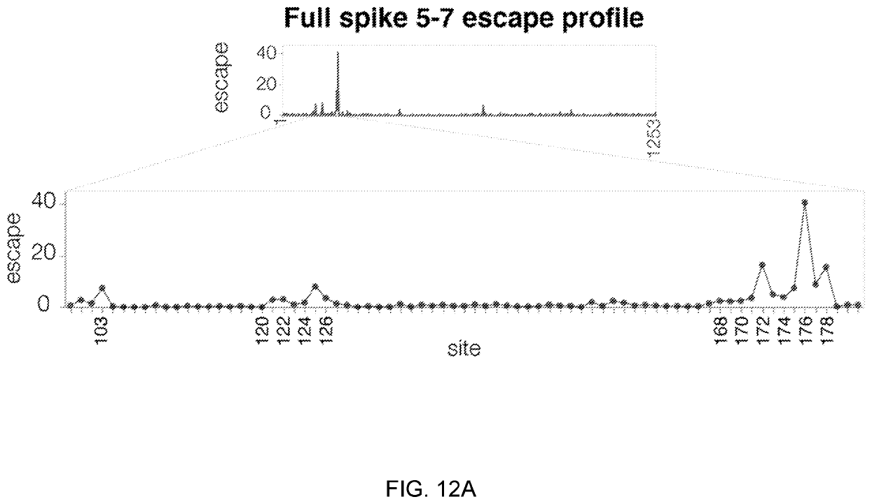

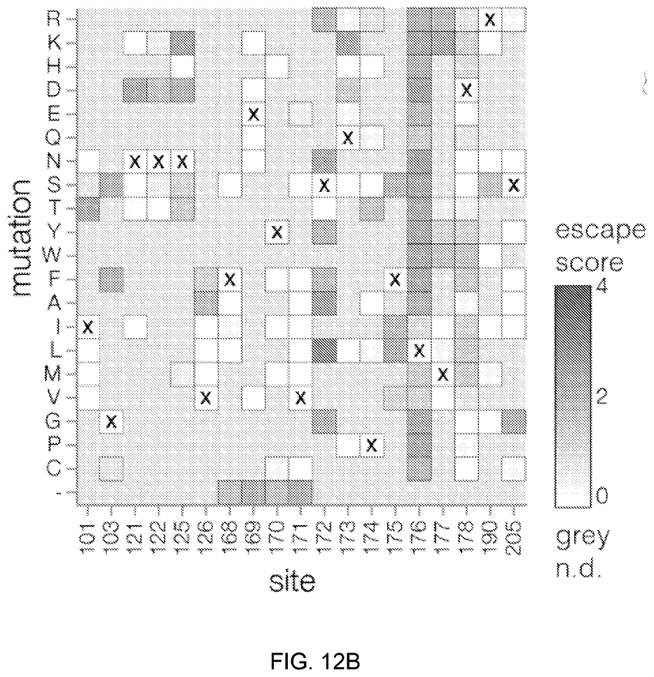

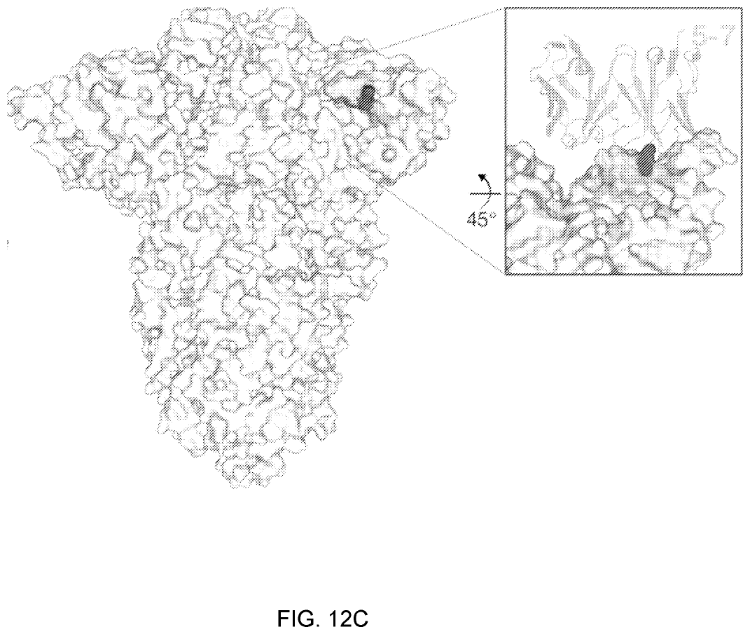

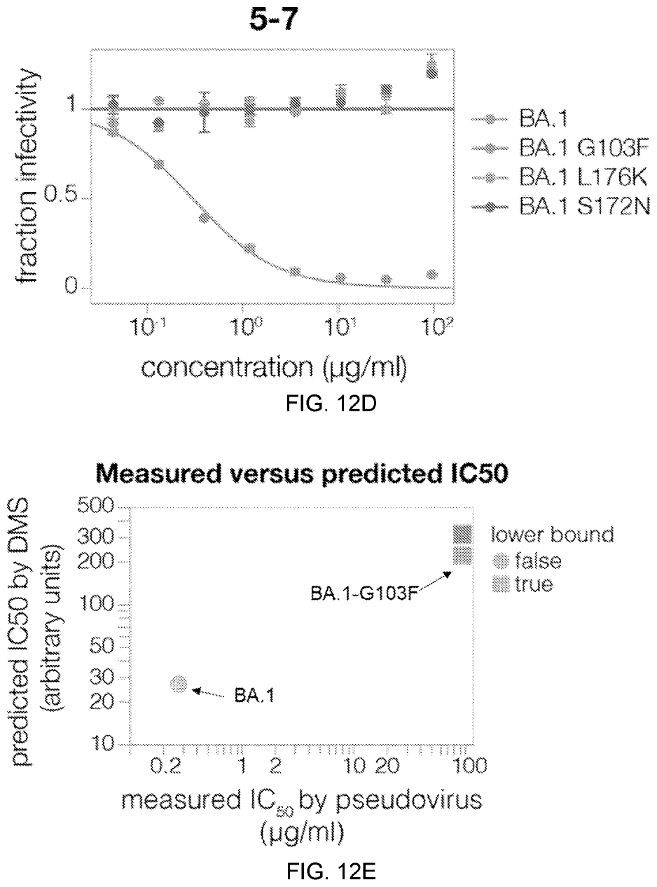

To show that escape from non-RBD-targeting antibodies can be mapped using full-spike deep mutational scanning, the NTD-targeting 5-7 antibody was next mapped (Cerutti et al., 2021, Cell Rep. 37, 109928). This antibody targets an epitope outside the defined antigenic supersite in NTD and is one of the few NTD-targeting antibodies isolated pre-Omicron that still retains some potency against Omicron variants (Cerutti et al., 2021, Cell Rep. 37, 109928; Liu et al., 2022, Nature 602, 676-681; McCallum et al., 2021, Cell 184, 2332-2347.e16). The deep mutational scanning showed that the key escape sites for 5-7 were in a hydrophobic pocket next to the N4 loop (site 172-178) (FIGS. 12A-12C), consistent with prior structural characterization of this antibody's epitope (Cerutti et al., 2021, Cell Rep. 37, 109928). In addition, deletions in 167-171 β-sheet, as well as mutations at the base of the adjacent loops such as G103 and V126 also escaped antibody 5-7 (FIGS. 12A, 12B). This deep mutational scanning was validated by performing individual neutralization assays with pseudoviruses containing L176K, S172N, and G103F mutations (FIG. 12D), all of which had the expected effect of completely escaping neutralization (FIG. 12E).

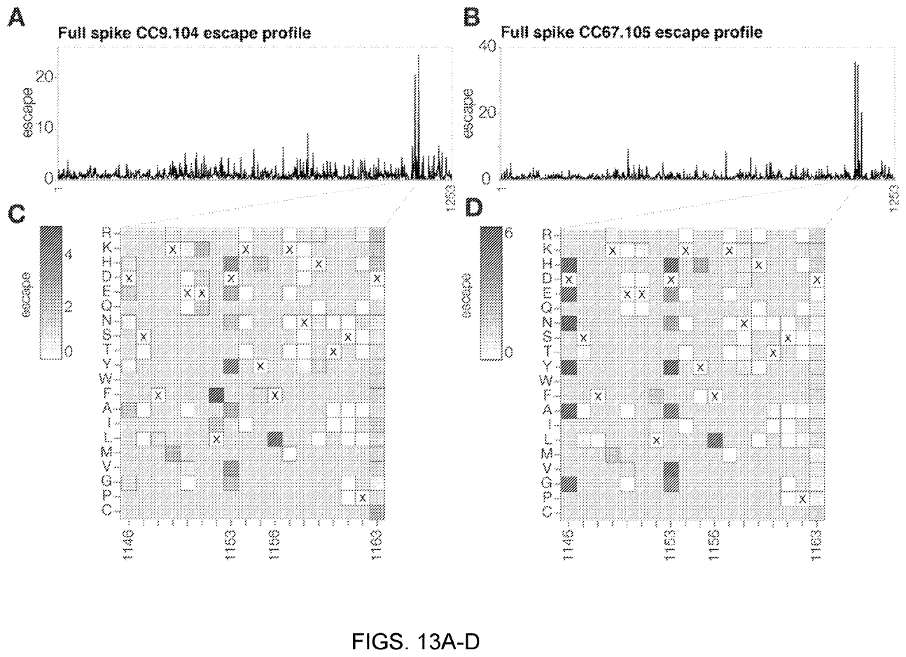

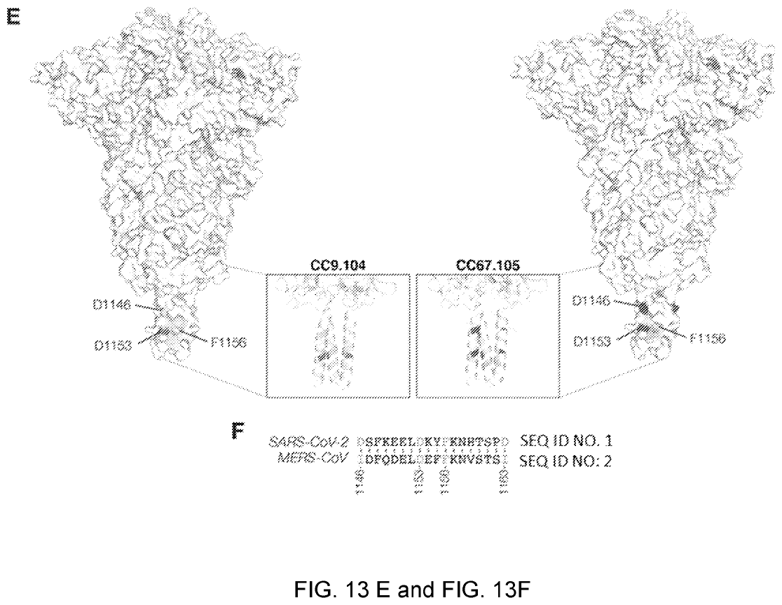

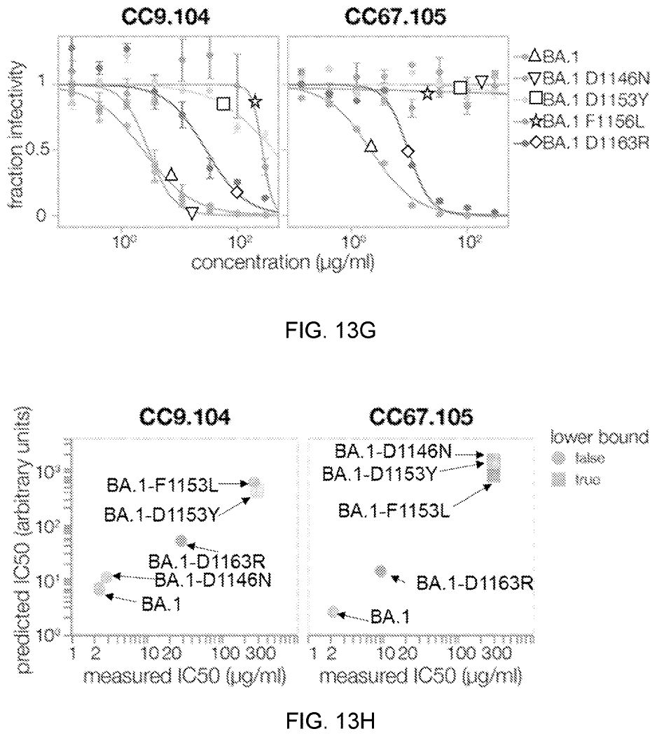

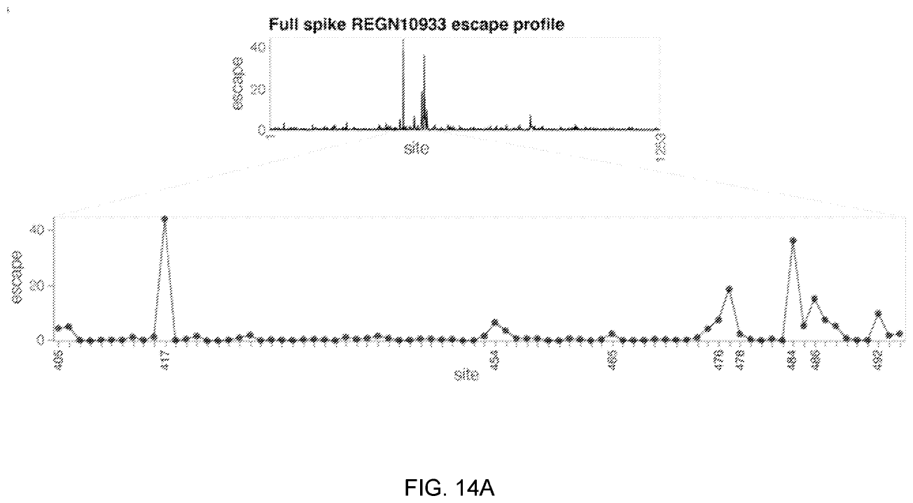

The full-spike deep mutational scanning was next applied to S2 domain-targeting antibodies CC9.104 and CC67.105, which were isolated using the conserved S2 stem-helix peptide as bait (Zhou et al., 2022, bioRxiv 2022.03.04.479488). Both CC9.104 and CC67.105 broadly neutralize SARS-related coronaviruses, and CC9.104 also retains some potency against Middle East respiratory syndrome coronavirus (MERS-CoV). As expected, the disclosed deep mutational scanning showed that escape sites for both antibodies cluster in the S2 stem helix region (FIGS. 13A-13E). The data also explains why only CC9.104 neutralizes MERS-CoV. The deep mutational scanning shows that the CC67.105 epitope centers on sites D1146, D1153, and F1156 (FIGS. 13B, 13D), and consistent with the deep mutational scanning, mutating these sites leads to complete escape in validation neutralization assays (FIG. 13G). By contrast, the deep mutational scanning shows that while CC9.104's epitope also includes sites D1153 and F1156, mutations at site D1146 cause only modest escape (FIGS. 13C, 13E), and validation neutralization assays again confirm these deep mutational scanning results (FIG. 13G). Notably, sites D1153 and F1156 are conserved between SARS-CoV-2 and MERS-CoV S2 stem-helix regions, but site D1146 is mutated to isoleucine in MERS-CoV (FIG. 13F). Therefore, while the change at D1146 to isoleucine completely escapes CC67.105 mAb it does not substantially impact neutralization by CC9.104 (FIG. 13G). Note that site D1163 is also mutated to isoleucine in MERS-CoV and both antibodies show some escape at that site, which may explain why CC9.104's potency against MERS-CoV is lower than against SARS-CoV-2.

The above deep mutational scanning of escape from the S2 antibodies emphasizes the difference between SARS-related coronavirus breadth and resistance to escape in SARS-CoV-2. Both CC9.104 and CC67.105 neutralize many diverse SARS-related coronaviruses, but Omicron sub-variants with mutations that completely escape these antibodies already exist (e.g. D1153Y in BA.2.46 and BA.2.59). Therefore, even pan-sarbecovirus neutralizing antibodies can be escaped by mutational diversity within SARS-CoV-2, which emphasizes the importance of directly mapping escape mutations in SARS-CoV-2 in addition to assessing breadth across other natural SARS-related coronaviruses.

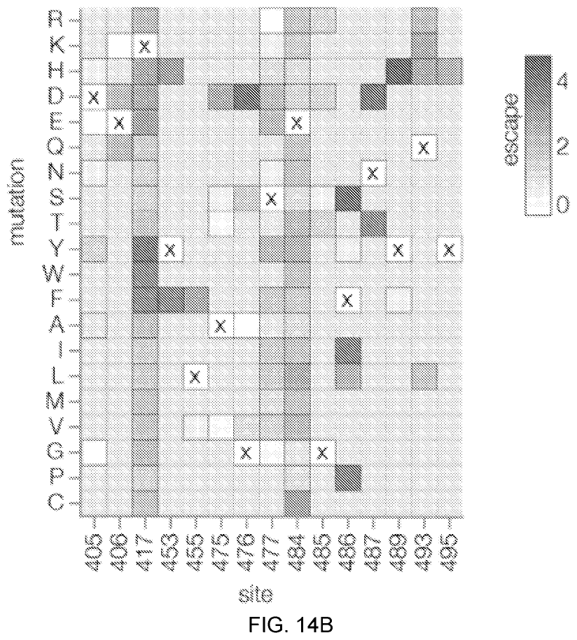

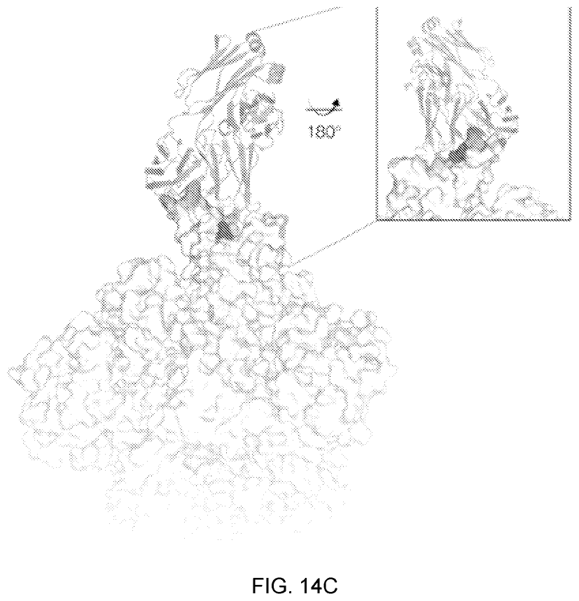

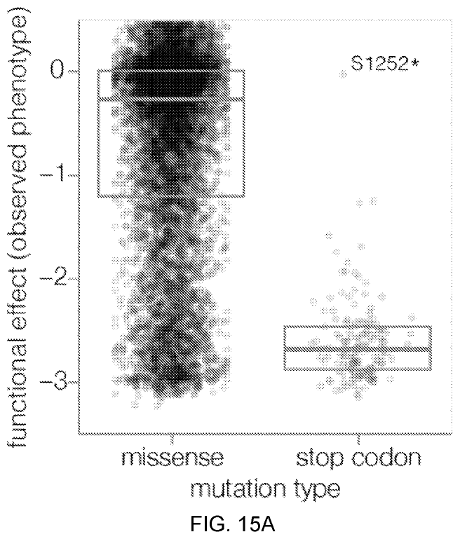

To show that deep mutational scanning of the spikes from different SARS-CoV-2 strains can be performed, escape from the REGN10933 antibody using Delta spike deep mutational scanning libraries was mapped (FIG. 14A, 14B). REGN10933 is a class 1 antibody that directly competes with ACE2 binding, and was part of the REGN-COV2 therapeutic cocktail used early in the pandemic but has lost potency against Omicron variants (Baum et al., 2020, Science eabd0831; Hansen et al., 2020, Science 369, 1010-1014; Liu et al., 2022, Nature 602, 676-681). Escape sites for REGN10933 mapped with the disclosed deep mutational scanning system overlapped with the antibody binding footprint and included previously described escape mutations (FIG. 14C) (Baum et al., 2020, Science eabd0831; Hansen et al., 2020, Science 369, 1010-1014; Starr et al., 2021, Science 371, 850-854).

To estimate the effects of individual mutations from the library measurements including both singly and multiply mutated Envs, a biophysical model in which antibody neutralization has a Hill-curve dependence on antibody concentration and mutations with a given epitope have additive effects on antibody was used. The model, which is implemented in the polyclonal software (jbloomlab.github.io/pouclonal/) utilizes information from both singly and multiply mutated Env variants under realistic assumptions about how mutations combine to escape antibody binding.

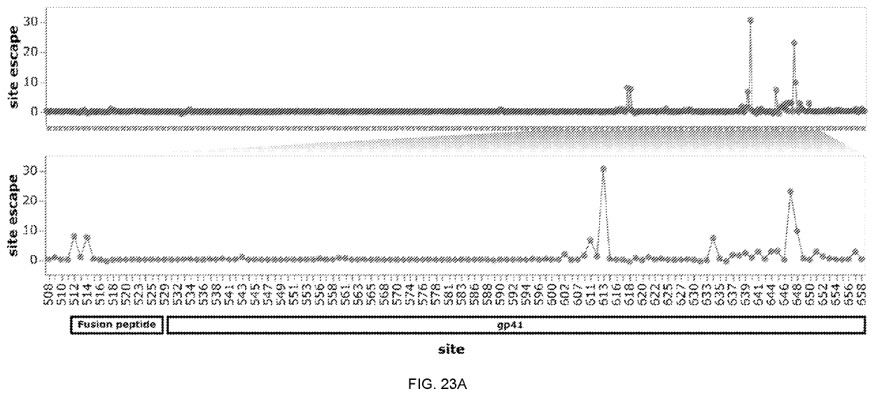

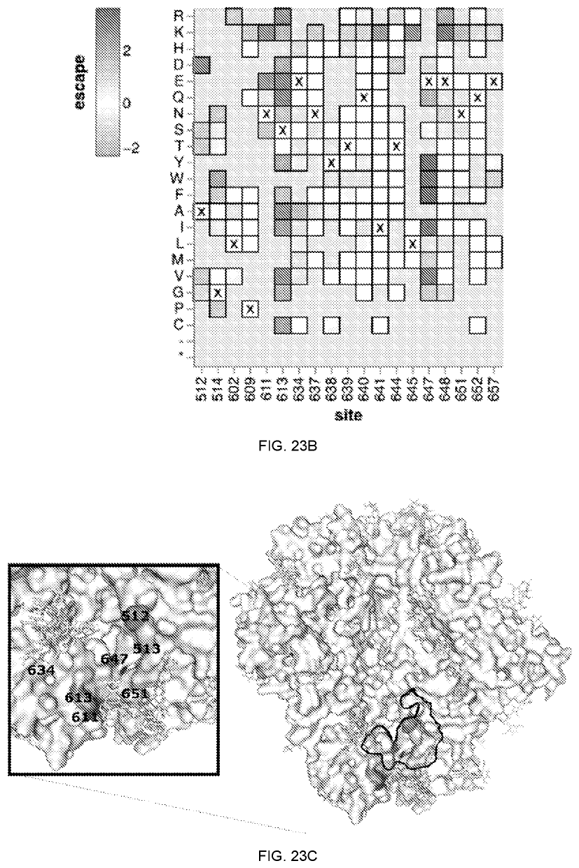

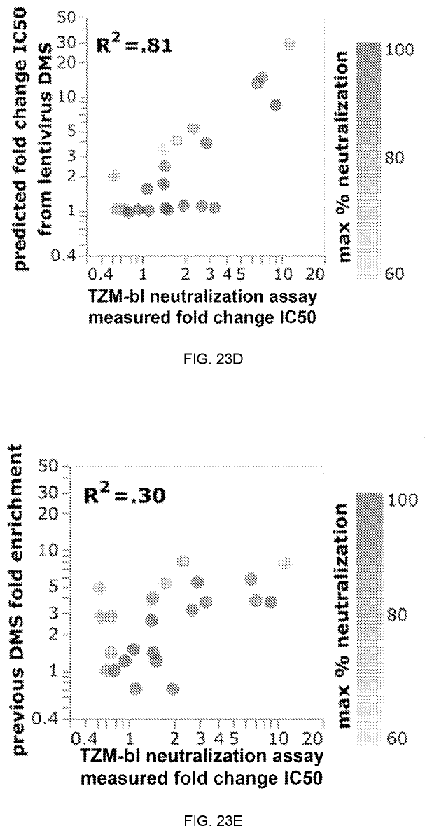

Mapping showed that PGT151 is escaped by Env mutations in the fusion peptide or affecting N-linked glycans recognized by PGT151 (FIGS. 23A and 23B). In particular, PGT151 is strongly escaped by any mutations knocking out the N611 glycan, specification mutations at the N637 glycan, mutations at sites 647 and 648, and mutations at sites 512 and 514 in the fusion peptide (FIGS. 23A and 23B). Lower magnitude escape was mapped at sites 537-543. All of these mutations are in or near the binding footprint PGT151 (FIG. 23C). The escape predicted by the deep mutational scanning was highly correlated with IC50s measured in previously performed TZM-bl neutralization assays (FIG. 23D). The correlation with neutralization IC50s was substantially better for the current deep mutational scanning than an earlier approach that used libraries of HIV virions in a system in which it was not possible to measure multiple mutations or absolute neutralization (FIGS. 23D and 23E).

Analysis of functional effects of mutations on spike-mediated pseudovirus infection. The disclosed deep mutational scanning also enables measurement of how mutations affect spike-mediated viral infection in the absence of antibodies. These measurements can be made by computing a functional score for each variant from its relative frequency in infectious spike pseudotyped lentiviruses generated from the disclosed single-copy cell integrated cells versus VSV G pseudotyped lentivirus generated from the same cells (FIG. 3C). Spike variants with negative functional scores are worse at mediating cellular infection than the parental unmutated spike, while variants with positive functional scores are better at mediating infection (FIG. 7). To deconvolve the functional scores for the variants (which often contain multiple mutations) into the effects of individual mutations on spike-mediated entry, global epistasis models were used (Otwinowski et al., 2018, Proc. Natl. Acad. Sci. 115, E7550-E7558).

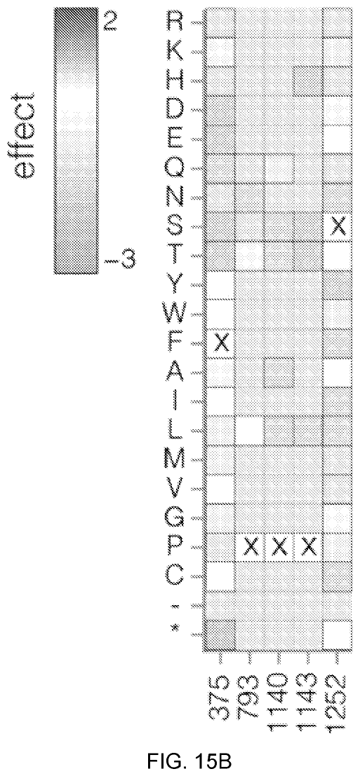

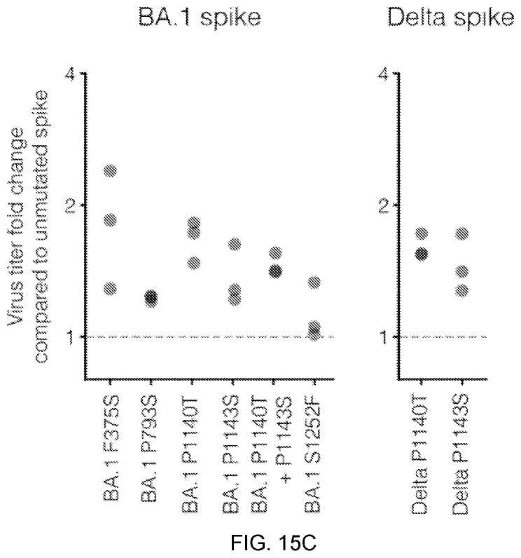

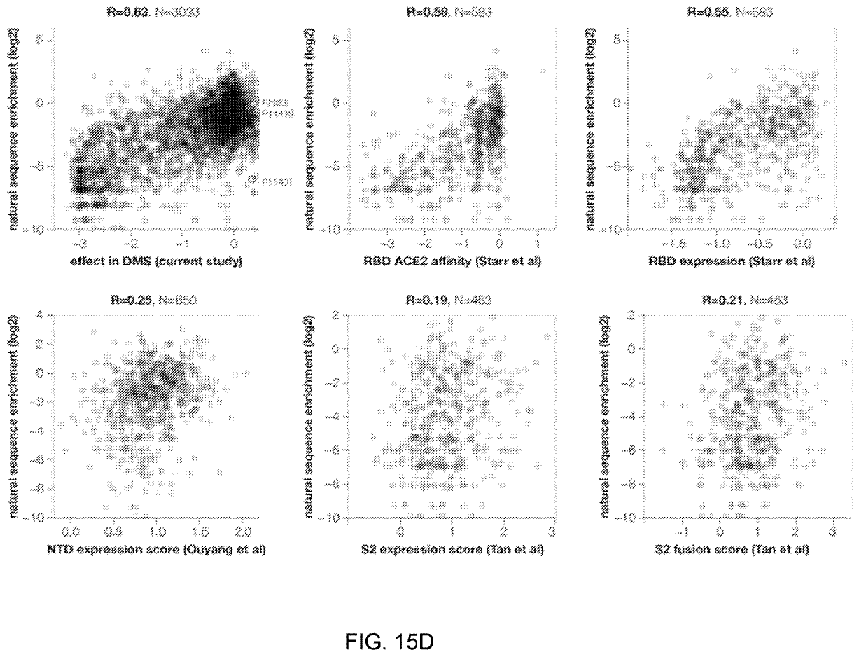

As expected stop-codon mutations to the BA.1 spike were highly deleterious for spike-mediated infection, whereas amino-acid mutations showed a wide range of effects ranging from slightly beneficial to roughly neutral to highly deleterious (FIG. 15A; recall that the disclosed library design excludes many of the most deleterious amino-acid mutations). To test whether the mutations measured to have slightly beneficial effects improved spike-mediated infection, five mutations that the deep mutational scanning indicated improved infection (FIG. 15B) were chosen, and pseudovirus mutants carrying these mutations were generated. The validation experiments confirmed that all the tested mutations indeed slightly improved spike-mediated infection (FIG. 15C), validating that the disclosed deep mutational scanning can identify mutations that increase spike-mediated pseudovirus infection.

To examine the relationship between the functional effects of spike mutations in the deep mutational scanning and the actual evolution of human SARS-CoV-2, the extent that mutations are enriched or depleted across a phylogenetic tree of all publicly available human SARS-CoV-2 sequences was determined (Turakhia et al., 2021, Nat. Genet. 53, 809-816). To do this, the number of independent observations of each mutation was calculated on the tree and these observed numbers were compared to the expected numbers under neutrality as estimated from four-fold synonymous sites, analyzing only mutations expected to have ≥20 occurrences (see Methods for details). The disclosed deep mutational scanning measurements of the effects of mutations on spike-mediated infection were reasonably correlated with the enrichment of mutations among actual sequences (FIG. 15D), indicating the disclosed experiments at least partially reflect the functional selection actually shaping spike evolution. A similar analysis for prior spike deep mutational scanning using yeast display of the RBD (Starr et al., 2022, doi.org/10.1101/2022.09.20.508745), or mammalian cell display of the NTD (Ouyang et al., 2022, bioRxiv 2022.06.20.496903) or a region of S2 (Tan et al., 2022, bioRxiv 2022.09.24.509341) (FIG. 15D) was performed. The disclosed pseudovirus-based spike deep mutational scanning measurements were more correlated with the enrichment of mutations during actual evolution than any of these prior cell-surface display deep mutational scanning studies, presumably because the disclosed experiments mimic the true biological function of spike better than cell-surface display experiments.

However, none of the mutations with positive deep mutational scanning functional scores that were validated to improve spike-mediated infection in a pseudovirus context are enriched during actual SARS-CoV-2 evolution (FIG. 15D). This is because there is some divergence between the selection pressure in the disclosed pseudovirus-based experiments and true natural selection on spike. For instance, mutations at sites P1140 and P1143, which are located at the beginning of the S2 stem-helix, could potentially destabilize the prefusion trimer leading to more rapid cell entry in a pseudovirus context but negatively affecting spike stability in the context of actual human transmission. Nonetheless, the disclosed functional measurements still provide the most accurate large-scale measurements to date on the effects of mutations to spike and should be useful for assessing which antibody-escape mutations are well enough tolerated to pose a plausible risk of emerging naturally. The disclosed experiments indicate that there are no further mutations to the BA.1 spike that improve pseudovirus titers to the same extent as the D614G mutation that fixed early in SARS-CoV-2's evolution in humans (Benton et al., 2021, Proc. Natl. Acad. Sci. 118, e2022586118; Plante et al., 2021, Nature 592, 116-121; Zhang et al., 2021, Science 372, 525-530).

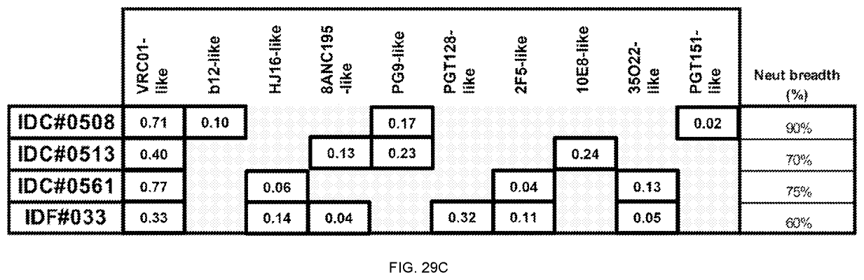

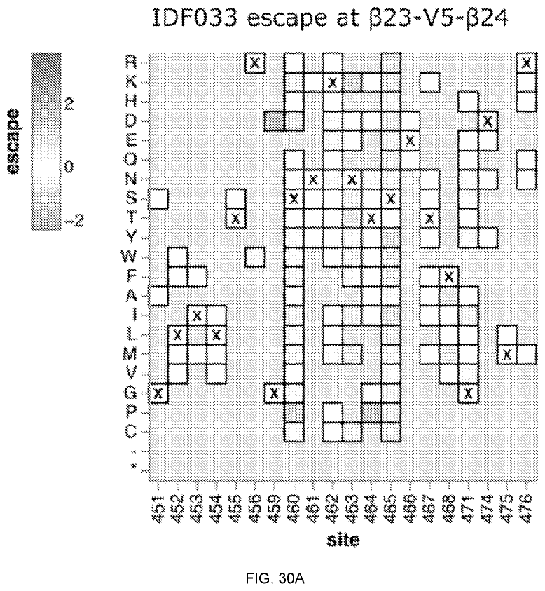

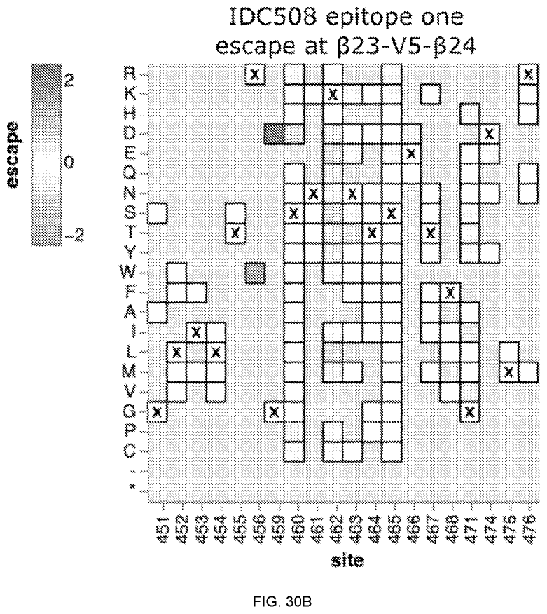

Analysis of functional effects of mutations on Env-mediated pseudovirus infection. In order to map neutralizing specificities in a polyclonal context, sera were selected based on their ability to broadly neutralize a global HIV panel and potently neutralize BF 520 pseudovirus. Based on these criteria, four sera were collected from individuals in Germany living with HIV: two with clade B viruses and two with clade D viruses (FIGS. 29A and 29B). Based on the f61 neutralizing fingerprinting panel, these sera were predicted to be primarily VRC01 like, meaning they target the CD4-binding site (FIG. 29C). Purified IgGs were used to ensure the removal of antiretroviral drugs.

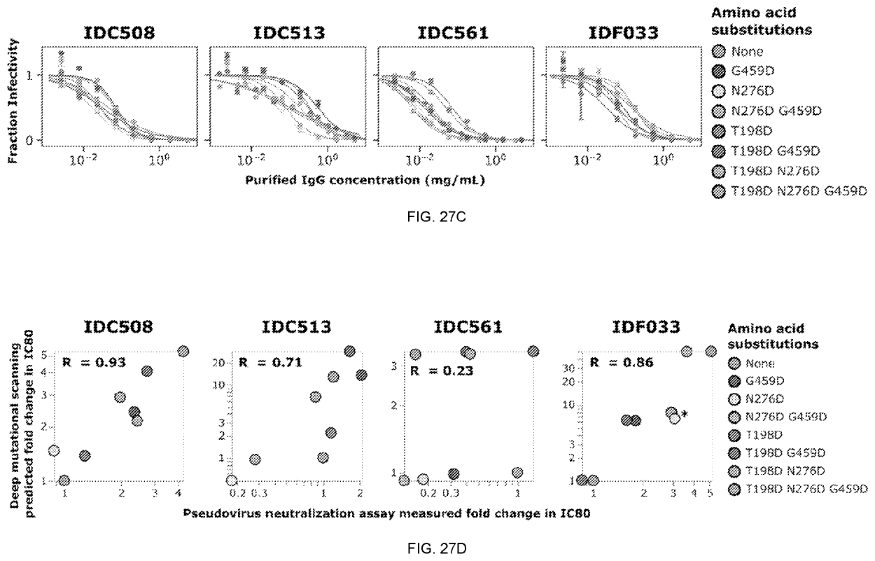

The results of the deep mutational scanning were validated by performing pseudovirus neutralization assays on single amino-acid mutants of Env with a range of effects in the escape maps. The changes in pseudovirus neutralization assay IC802 correlated well with the mutational effects predicted by the deep mutational scanning for all four sera (FIGS. 27A and 27B).

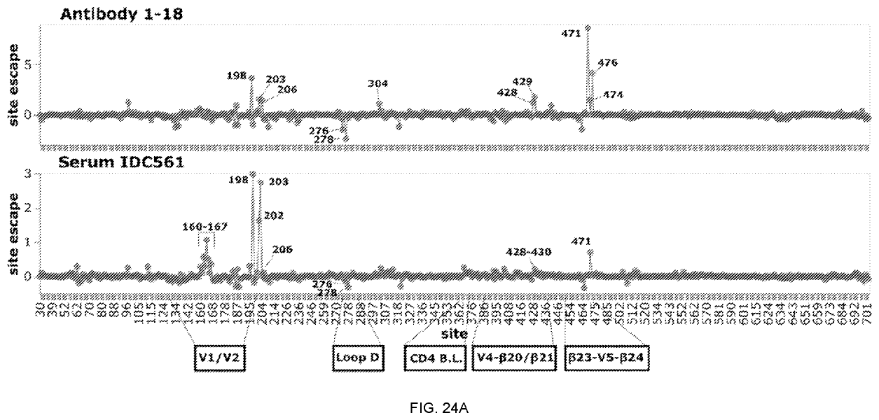

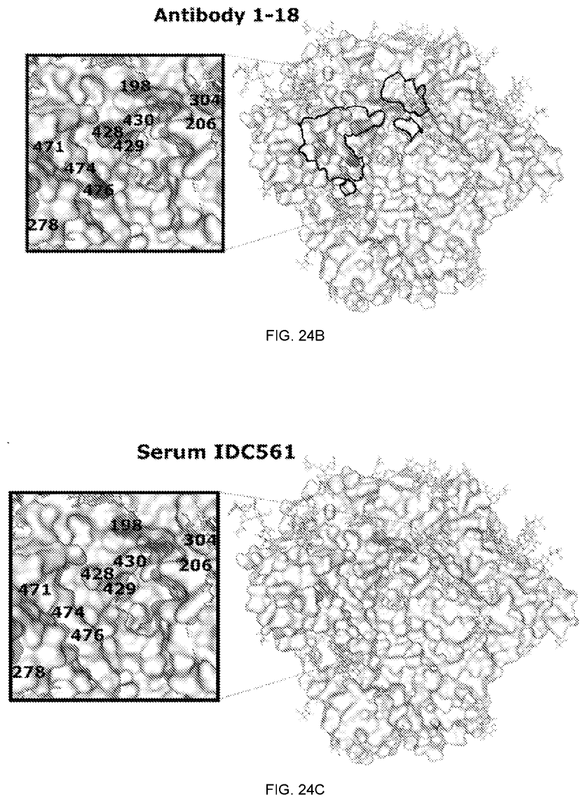

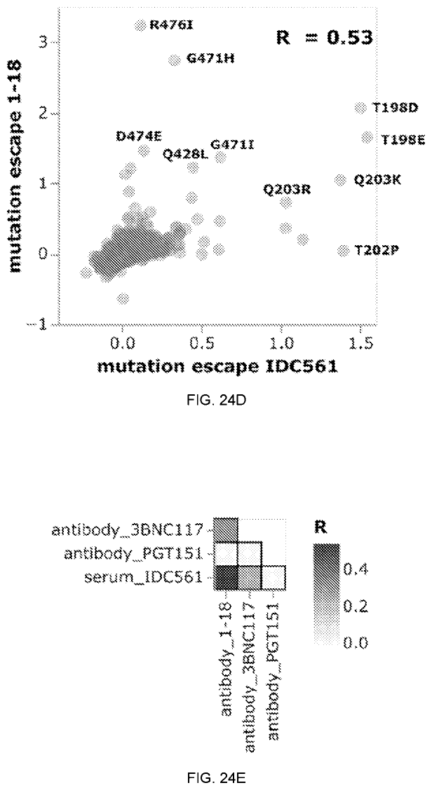

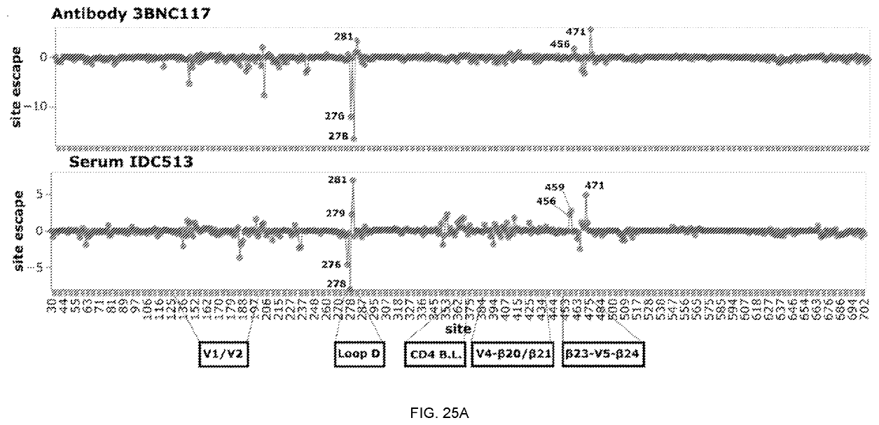

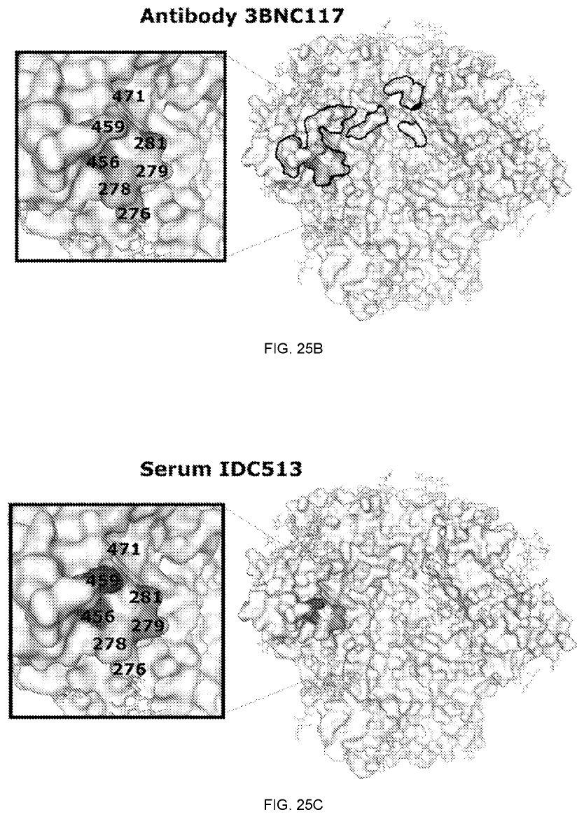

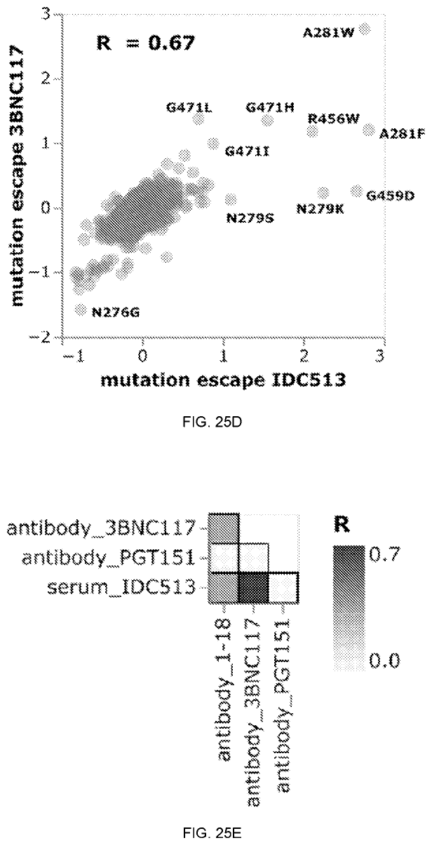

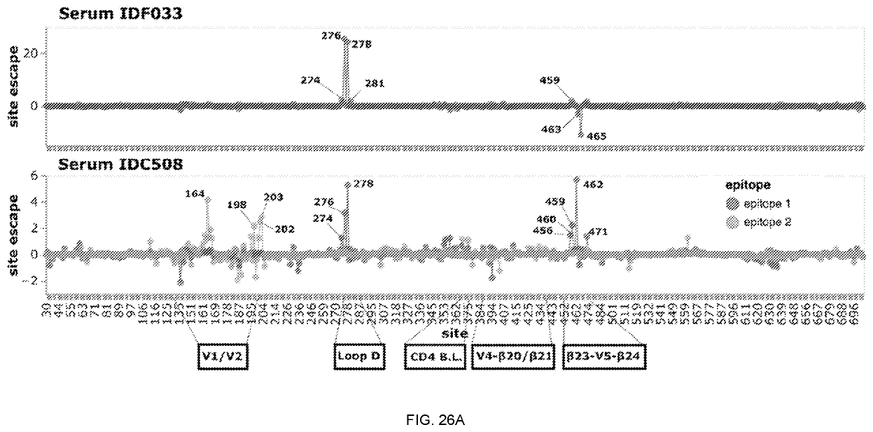

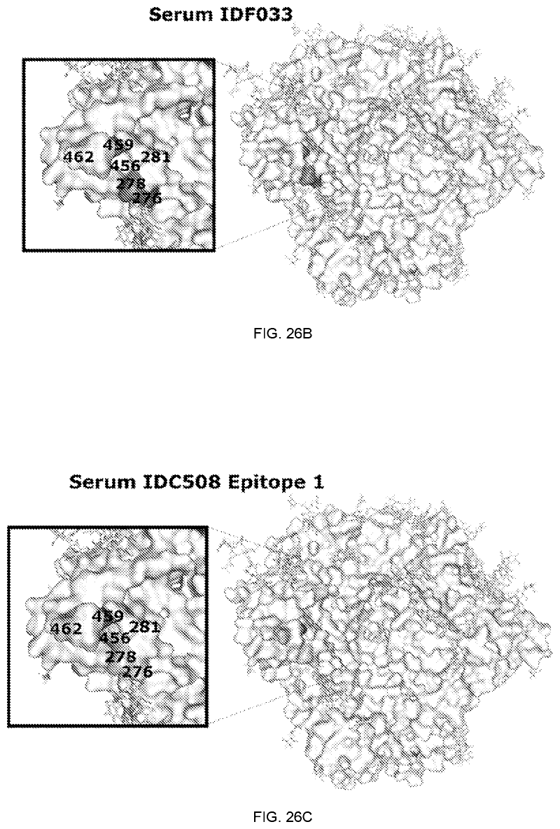

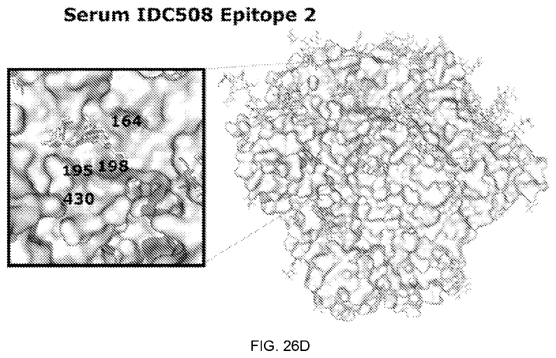

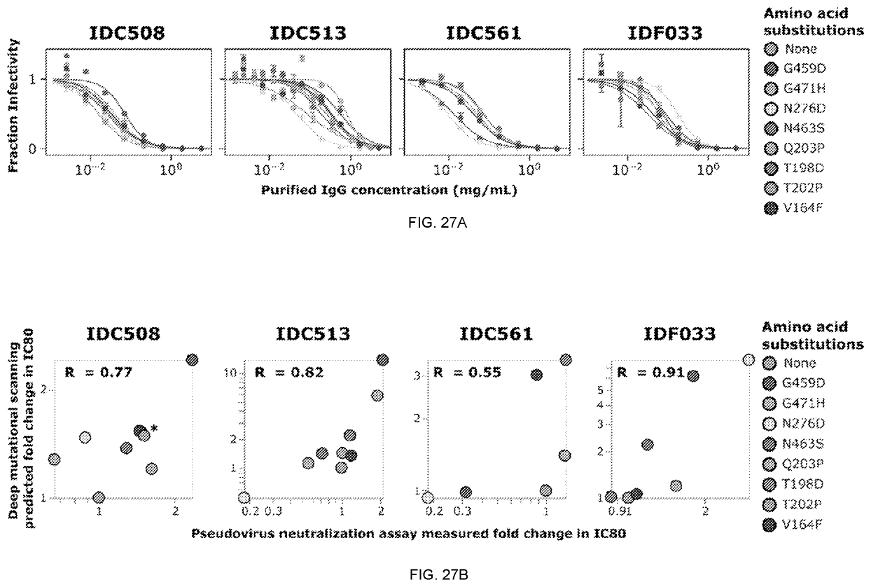

The correlation between the deep mutational scanning and neutralization assays was particularly good for strong escape mutations. For every serum, the tested mutations predicted to escape neutralization most strongly by the deep mutational scanning increased the neutralization assays IC80 (FIGS. 27A and 27B). The correlation was less consistent for mutations that enhanced neutralization sensitivity rather than escape. For instance, N276D for serum IDC561 caused greater enhancement of neutralization sensitivity in the neutralization assays than predicted from the deep mutational scanning (FIGS. 27A and 27B). The reduced accuracy of the deep mutational scanning for identifying sensitizing mutations is likely because the mapping experiments were performed at relatively higher serum concentrations making them better suited to identify escape rather than sensitizing mutations. Good correlation was also found for Env variants with combinations of mutations as shown in FIGS. 27C and 27D. The deep mutational scanning mapped IDC508 to have two epitopes and pseudovirus neutralization assays using combinations of mutations supports this prediction. Specifically, T198D is in one epitope of IDC508, whereas N276D and G459D are in the other epitope (FIG. 26A)—and as predicted, N276D and G459D each cause more escape when combined with T198D (FIGS. 27C and 27D). Note, however, that the effects of these combinations of mutations are complex because N276D has some sensitizing effect on its own (FIGS. 27C and 27D). The greatest escape from IDC508 is caused by combining all three of T198D, N276D, and G459D, suggesting that this combination escapes a substantial fraction of the neutralizing antibodies in the serum. For sera IDC513 and IDC561, the deep mutational scanning predicted that combinations of mutations would not have substantially more escape than the single mutations with the highest effect, and this was validated in neutralization assays (FIGS. 27C and 27D). Deep mutational scanning mapped IDC513 and IDC561 to each have one epitope (FIGS. 24A and 25A). As expected for sera that target a single epitope, no combinations of mutations caused higher fold change IC80 in neutralization assays than the best escaping single mutations for IDC513 and IDC561. For IDC561, only one mutation tested in combinations (T198D) was measured in the deep mutational scanning to be an escape mutation with the others being sensitizing mutations. Consistent with the deep mutational scanning, only the T198D single mutant caused escape in the neutralization assays. Despite being a sensitizing mutation for some sera, deep mutational scanning predicted mutations to site 276 to cause strong escape from serum IDF033 (FIG. 26A), and as expected N276D caused a large increase in neutralization assay IC80 both alone and in combination with other mutations (FIGS. 27A-27D). Consistent with the deep mutational scanning, combining N276D with another strong escape mutation, G459D, further increased the neutralization assay ICD80 (FIGS. 27C and 27D). The mapping shows that although the sera all target Env's CD4 binding site, they differ markedly in the actual epitopes that are the focus of the neutralizing response.

The foregoing discussion describes the development of a new deep mutational scanning system for assessing the antigenic and functional effects of mutations in the SARS-CoV-2 spike and HIV viral envelope proteins. This deep mutational scanning system is the first to measure how mutations to the entirety of spike affect cellular infection and therefore enables the mapping of escape from antibodies targeting any part of the spike. Further, this system allows for the measurement of combinations of mutations, enabling more effective mapping of escape from polyclonal serum that may be target multiple epitopes. The disclosed system directly measures how mutations affect antibody neutralization and shows that these measurements correlate well with traditional pseudovirus neutralization assays. The ability to directly measure neutralization as opposed to binding will be especially useful when applied to polyclonal sera since the magnitude of how mutations affect neutralization versus binding can differ in a polyclonal context (Cao et al., 2022, bioRxiv 2022.09.15.507787).

Here the use of this new deep mutational scanning system to map escape from monoclonal antibodies using libraries based on different SARS-CoV-2 variants was described. Escape from antibodies was mapped targeting RBD, NTD, and S2 domains of spike. It was shown how mutation-level escape mapping can be used to predict the ability of emerging variants to escape therapeutic antibodies (such as Ly-CoV1404). In addition, using S2 stem-helix binding antibodies it was shown how the disclosed deep mutational scanning system can be used to assess the trade-off between antibody breadth and within variant escapability. The same approach can be used to map escape from polyclonal antibody mixtures such as vaccinated and convalescent sera from individuals.

The deep mutational scanning system was also used to map Env mutations that escape antibody neutralization using mutant libraries. The mapping showed that PGT151 is escaped by mutations in the fusion peptide or affecting N-inked glycans recognized by PGT151 (FIGS. 23A and 23B). Strong effects of mutations were observed at the N276 glycan for several broad and potent sera targeting the CD4 binding site, suggesting this glycan may be important to vaccination strategies. Maps of escapes from neutralizations of combinations of broadly neutralizing antibodies targeting different regions of Env could aid in antibody selection. Vaccine-elicited sera can also be mapped to evaluate experimental vaccines and compare their neutralization activity with known broadly neutralizing antibodies or sera. The method described here can thus be used to inform the design of both therapeutics and vaccines.

The new deep mutational scanning system can be straightforwardly extended to any virus with an entry protein amenable to lentiviral pseudotyping. This set of viruses includes other coronaviruses, influenza viruses, filoviruses, arenaviruses, and henipaviruses-all of which have receptor-binding and fusion proteins for which lentiviral pseudotyping provides a safe way to study cellular infection and antibody neutralization without requiring direct work with the actual pathogenic virus (Huang et al., 2020, Biomed. J. 43, 375-387; Khetawat and Broder, 2010, Virol. J. 7, 312; Kobinger et al., 2001, Nat. Biotechnol. 19, 225-230; Larson et al., 2008, J. Virol. 82, 10768-10775; Medina et al., 2003, Mol. Ther. 8, 777-789). Deep mutational scanning of the entry proteins of all these viruses could provide valuable information for antigenic surveillance and vaccine design since these proteins are the dominant target of neutralizing antibodies. However, data generated by such a system could be used to inform the introduction of gain-of-function mutations into actual potential pandemic viral pathogens. With the highly pathogenic avian influenza viruses, such as the H5N1 virus and H7N9 virus, preserving poly-basic furin cleavage sites in experiments can be used for biosafety. Advances in the high-throughput characterization of mutations to viral proteins should be coupled with thoughtful limits on any downstream experiments with actual replicating viruses (Inglesby et al., 2022, www.centerforhealthsecurity.org/news/center-news/pdfs/220629-RecstostrengthenUSGePPPand DURCPolicies.pdf) to ensure that safely generated information is used to benefit public health without creating new risks.

Having described in detail this exemplary use of the new mutational scanning libraries, additional optional uses and components are now described.

In particular embodiments, a deep mutational scanning library includes variants with 19 possible amino acid substitutions at each amino acid position and all possible codons of the associated 63 codons at each amino acid position. In particular embodiments, a deep mutational scanning library includes variants with every possible codon substitution at every amino acid position in a gene of interest with one codon substitution per library member. In particular embodiments, a deep mutational scanning library includes variants with one, two, or three nucleotide changes for each codon at every amino acid position in a gene of interest with one codon substitution per library member. In particular embodiments, a deep mutational scanning library includes variants with one, two, or three nucleotide changes for each codon at two amino acid positions, at three amino acid positions, at four amino acid positions, at five amino acid positions, at six amino acid positions, at seven amino acid positions, at eight amino acid positions, at nine amino acid positions, at ten amino acid positions, etc., up to at all amino acid positions, in a gene of interest with one codon substitution per library member. In particular embodiments, the start codon is not mutagenized. In particular embodiments, the start codon is Met.

In particular embodiments, a deep mutational scanning library includes variants with one, two, or three nucleotide changes for each codon at every amino acid position in a gene of interest with more than one codon substitution, more than two codon substitutions, more than three codon substitutions, more than four codon substitutions, or more than five codon substitutions, per library member. In particular embodiments, a deep mutational scanning library includes variants with one, two, or three nucleotide changes for each codon at every amino acid position in a gene of interest with up to all codon substitutions per library member. In particular embodiments, 20% of library members can be wildtype, 35% can be single mutants, and 45% can be multiple mutants. Multiple mutants can be advantageous, and the sequencing required by the systems and methods disclosed herein is so efficient that using 20% of reads on wild type is not a problem. Additionally, there are alternative (more complex) mutagenesis methods that give a larger proportion of single amino acid mutants [see, e.g., Kitzman, et al. (2015) Nature Methods 12: 203-206; Firnberg & Ostermeier (2012) PLoS One 7: e52031; Jain & Varadarajan (2014) Analytical Biochemistry 449: 90-98; and Wrenbeck, et al. (2016) Nature Methods 13: 928].

In particular embodiments, a deep mutational scanning library includes or encodes all possible amino acids at all positions of a protein, and each variant protein is encoded by more than one variant nucleotide sequence. In particular embodiments, a deep mutational scanning library includes or encodes all possible amino acids at all positions of a protein, and each variant protein is encoded by one nucleotide sequence. In particular embodiments, a deep mutational scanning library includes or encodes all possible amino acids at less than all positions of a protein, for example at 10%, 20%, 30%, 40%, 50%, 60%, 70%, 80%, 90%, 95%, 96%, 97%, 98% or 99% of positions. In particular embodiments, a deep mutational scanning library includes or encodes less than all possible amino acids (for example 10%, 20%, 30%, 40%, 50%, 60%, 70%, 80%, 90%, 95%, 96%, 97%, 98% or 99% of potential amino acids) at all positions of a protein. In particular embodiments, a deep mutational scanning library includes or encodes less than all possible amino acids (for example 10%, 20%, 30%, 40%, 50%, 60%, 70%, 80%, 90%, 95%, 96%, 97%, 98% or 99% of potential amino acids) at less than all positions of a protein, for example at 10%, 20%, 30%, 40%, 50%, 60%, 70%, 80%, 90%, 95%, 96%, 97%, 98% or 99% of positions. In particular embodiments, a deep mutational scanning library including a set of variant nucleotide sequences can collectively encode protein variants including at least a particular number of amino acid substitutions at at least a particular percentage of amino acid positions. “Collectively encode” takes into account all amino acid substitutions at all amino acid positions encoded by all the variant nucleotide sequences in total in a deep mutational scanning library.

In particular embodiments, a codon mutant library can be synthetically constructed by and obtained from a synthetic DNA company such as Twist Bioscience (San Francisco, CA). In particular embodiments, methods to generate a codon mutant library include: nicking mutagenesis as described in Wrenbeck et al. (2016) Nature Methods 13: 928-930 and Wrenbeck et al. (2016) Protocol Exchange doi:10.1038/protex.2016.061; PFunkel (Firnberg & Ostermeier (2012) PLoS ONE 7(12): e52031); massively parallel single-amino-acid mutagenesis using microarray-programmed oligonucleotides (Kitzman et al. (2015) Nature Methods 12: 203-206); and saturation editing of genomic regions with CRISPR-Cas9 (Findlay et al. (2014) Nature 513(7516): 120-123).

Examples of inducible promoter systems that can be used in the systems and methods of the present disclosure include: lac operon [Brown et al. (1987) Cell 49: 603-612; Hu and Davidson (1987) Cell 48: 555-566]; tetracycline (Tet) (or derivative doxycycline)-inducible systems (Tet-On and Tet-Off) [Gossen et al. (1995) Science 268: 1766-1769; Baron et al. (1997) Nucleic Acids Res 25: 2723-2739; Blau and Rossi (1999) Proc Natl Acad Sci USA 96: 797-799]; mifepristone-inducible systems (GeneSwitch) [Burcin et al. (1999) Proc. Natl. Acad. Sci. USA 96(2): 355-360; Wang et al. (1994) Proc. Natl. Acad. Sci. USA 91(17): 8180-8184]; ecdysone-regulated system [Galimi et al. (2005) Blood 105(6): 2400-2402]; streptogramin-adjustable expression system derived from Streptomyces coelicolor [Mitta et al. (2004) Nucleic Acids Res 32(12): e106]; gaseous acetaldehyde-inducible expression system derived from Aspergillus nidulans [Hartenbach S & Fussenegger M (2005) J Biotechnol 120(1): 83-98]; and cumate-inducible systems [U.S. Pat. No. 7,745,592; Mullick et al. (2006) BMC Biotechnology 6:43].

Examples of constitutive promoters include CMV (Karasuyama et al. 1989. J. Exp. Med. 169:13), ubiquitin, beta-actin (Gunning et al. 1989. Proc. Natl. Acad. Sci. USA 84:4831-4835) and pgk (see, for example, Adra et al. 1987. Gene 60:65-74; Singer-Sam et al. 1984. Gene 32:409-417; and Dobson et al. 1982. Nucleic Acids Res. 10:2635-2637).

“Encoding” refers to the property of specific sequences of nucleotides in a gene, such as a cDNA, or an mRNA, to serve as templates for the synthesis of other macromolecules such as a defined sequence of amino acids.

Polynucleotide gene sequences encoding more than one portion of an expressed binding domain molecule can be operably linked to each other and relevant regulatory sequences. For example, there can be a functional linkage between a regulatory sequence and an exogenous nucleic acid sequence resulting in expression of the latter. For instance, a promoter is operably linked to a coding sequence if the promoter affects the transcription or expression of the coding sequence.

In particular embodiments, a functional 3′LTR includes a functional U3. A functional 3′LTR can be obtained by repairing a deleted or disrupted 3′ LTR from a self-inactivating (SIN) lentiviral system that disrupts genome packaging (Miyoshi et al. (1998) Journal of Virology 72(10): 8150-8157). The repair can include cloning the 5′ LTR into the correct location at the 3′. These SIN retroviral systems include pHAGE, pHAGE2, and other pHAGE systems (described in protocols by the Kotton Lab at the Center for Regenerative Medicine, Boston University), and pHIV and other variants such as pHIV-7 (Miyoshi et al. (1998), supra). In particular embodiments, a functional U3 can be obtained from LTRs of other retroviruses, such as murine leukemia virus (MLV). Moloney MLV (MoMLV) retroviral systems include replication-competent (functional) LTRs (Dalba et al. (2007) Molecular Therapy 15(3): 457-466). In particular embodiments, a functional U3 can be obtained from an LTR of a retrovirus belonging to the Retroviridae family. In particular embodiments, a functional U3 is a full U3 sequence. In particular embodiments, a functional 3′LTR is a 3′LTR from a retrovirus that has not been modified. In particular embodiments, a functional 3′LTR allows transcription of the integrated variant gene, reporter, selectable marker, and barcode, and subsequent packaging into retroviral particles. In particular embodiments, the inclusion of a functional U3 means that the integrated variant gene, selectable marker, and barcode are transcribed and packaged into retroviral particles when cells storing the library are additionally transfected with helper plasmids.

In particular embodiments, alternative envelope glycoproteins (GPs) that can be used instead of VSV G include MLV GP and feline endogenous retrovirus (RD114) GP, gibbon ape leukemia virus (GALV) Env, and variants of these. Cronin et al. (2005) Curr Gene Ther. 5(4): 387-398. In particular embodiments, GPs that can be used are derived from a family including Rhabdoviridae, Arenaviridae, Togaviridae, Filoviridae, Retroviridae, Coronaviridae, Paramyxoviridae, Flaviviridae, Orthomyxoviridae, and Baculoviridae. In particular embodiments, GPs that can be used are derived from a genus including Vesiculovirus, Lyssavirus, Arenavirus, Alphavirus, Filovirus, Alpharetrovirus, Betaretrovirus, Gammaretrovirus, Deltaretrovirus, Spumavirus, Lentivirus, Coronavirus, Respirovirus, Hepacivirus, Influenzavirus A, and nucleopolyhedrovirus. In particular embodiments, GPs that can be used are derived from a species including vesicular stomatitis virus (Indiana virus), Chandipura virus, rabies virus, Mokola virus, Lymphocytic choriomeningitis virus (LCMV), Ross River virus (RRV), Sindbis virus, Semliki Forest virus (SFV), Venezuelan equine encephalitis virus, Ebola virus Reston, Ebola virus Zaire, Marburg virus, Lassa virus, avian leukosis virus (ALV), Jaagsiekte sheep retrovirus (JSRV), MLV, GALV, RD114, human T-lymphotropic virus 1 (HTLV-1), human foamy virus, Maedi-visna virus (MVV), severe acute respiratory syndrome coronavirus (SARS-CoV), Sendai virus, Respiratory syncytial virus (RSV), human parainfluenza virus type 3, hepatitis C virus (HCV), influenza virus, fowl plague virus (FPV), and Autographa californica multiple nucleopolyhedro virus (AcMNPV).

In particular embodiments, the barcode is 18 nucleotides in length. In particular, embodiments, because there are 4¹⁸-7¹⁰ different 18-nucleotide sequences, virtually every variant can have a unique barcode. The barcode can be any appropriate length and composition that does not negatively affect the fitness of the encoded variant protein. In particular embodiments, the length of the barcode is based on the size of the deep mutation scanning library. If more distinct barcodes are needed, then barcodes of greater length can be used. If less distinct barcodes are needed, then barcodes of lesser length can be used. In particular embodiments, the barcode can be 5-100 nucleotides in length. In particular embodiments, the barcode can be 10-80 nucleotides in length. In particular embodiments, the barcode can be 10-50 nucleotides in length. In particular embodiments, the barcode can be 8-30 nucleotides in length. In particular embodiments, the barcode can be 12-24 nucleotides in length. In particular embodiments, the barcode can be 16-20 nucleotides in length. In particular embodiments, the barcode can be 3 nucleotides in length, 4 nucleotides in length, 5 nucleotides in length, 6 nucleotides in length, 7 nucleotides in length, 8 nucleotides in length, 9 nucleotides in length, 10 nucleotides in length, 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, 29 nucleotides in length, 30 nucleotides in length, 31 nucleotides in length, 32 nucleotides in length, 33 nucleotides in length, 34 nucleotides in length, 35 nucleotides in length, 36 nucleotides in length, 37 nucleotides in length, 38 nucleotides in length, 39 nucleotides in length, 40 nucleotides in length, or more.

In particular embodiments, the reporter is ZsGreen or green fluorescent protein (GFP). However, as is understood by those of ordinary skill in the art, any appropriate reporter or selectable marker can be used. Additional examples include blue fluorescent proteins (e.g. eBFP, eBFP2, Azurite, mKalamal, GFPuv, Sapphire, T-sapphire); cyan fluorescent proteins (e.g. eCFP, Cerulean, CyPet, AmCyanl, Midoriishi-Cyan); additional green fluorescent proteins (e.g. GFP-2, tagGFP, turboGFP, eGFP, Emerald, Azami Green, Monomeric Azami Green, CopGFP, AceGFP, ZsGreenl); orange fluorescent proteins (mOrange, mKO, Kusabira-Orange, Monomeric Kusabira-Orange, mTangerine, tdTomato); red fluorescent proteins (mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, Jred); yellow fluorescent proteins (e.g., YFP, eYFP, Citrine, Venus, YPet, PhiYFP, ZsYellowl); and any other suitable fluorescent proteins, including, for example, firefly luciferase. In particular embodiments, the reporter or selectable marker can include any cell surface displayed marker that can be detected with an antibody that binds to that marker and allows sorting of cells that have the marker. In particular embodiments, the reporter or selectable marker can include the magnetic sortable marker streptavidin binding peptide (SBP) displayed at the cell surface by a truncated Low Affinity Nerve Growth Receptor (LNGFRF) and one-step selection with streptavidin-conjugated magnetic beads (Matheson et al. (2014) PloS one 9(10): el 11437).

Particular embodiments can also utilize cerulenin resistance genes (e.g., fas2m, PDR4; Inokoshi et al., Biochemistry 64: 660, 1992; Hussain et al., Gene 101: 149, 1991); copper resistance genes (CUP1; Marin et al., Proc. Natl. Acad. Sci. USA. 81: 337, 1984); and geneticin resistance gene (G418r) as markers.

Additional useful selectable markers include β-galactosidase (β-gal) and β-glucuronidase (GUS) (see, e.g., European Patent Publication EP2423316). These reporter proteins function by hydrolyzing a secondary marker molecule (e.g., a β-galactoside or a β-glucuronide). Thus it will be understood that methods and systems that employ one of these marker proteins will also involve providing the compound(s) needed to produce a detectable reaction product. Assays for detecting β-gal or GUS activity are well known in the art.

In particular embodiments, it may be appropriate to use auxotrophic markers as reporters or selectable markers. Exemplary auxotrophic markers include methionine auxotrophic markers (e.g., met1, met2, met3, met4, met5, met6, met7, met8, met10, met13, met14 or met20); tyrosine auxotrophic markers (e.g., tyr1 or isoleucine); valine auxotrophic markers (e.g., ilv1, ilv2, ilv3 or ilv5); phenylalanine auxotrophic markers (e.g., pha2); glutamic acid auxotrophic markers (e.g., glu3); threonine auxotrophic markers (e.g., thr1 or thr4); aspartic acid auxotrophic markers (e.g., asp1 or asp5); serine auxotrophic markers (e.g., ser1 or ser2); arginine auxotrophic markers (e.g., arg1, arg3, arg4, arg5, arg8, arg9, arg80, arg81, arg82 orarg84); uracil auxotrophic markers (e.g., ura1, ura2, ura3, ura4, ura5 or ura6); adenine auxotrophic markers (e.g., ade1, ade2, ade3, ade4, ade5, ade6, ade8, ade9, ade12 or ade15); lysine auxotrophic markers (e.g., lys1, lys2, lys4, lys5, lys7, lys9, lys11, lys13 or lys14); tryptophan auxotrophic markers (e.g., trp1, trp2, trp3, trp4 or trp5); leucine auxotrophic markers (e.g., leu1, leu2, leu3, leu4 or leu5); and histidine auxotrophic markers (e.g., his1, his2, his3, his4, his5, his6, his7 or his8).

Particular embodiments of the libraries disclosed herein utilize pro- and/or anti-viral factors to make experimental environments more or less conducive to viral fitness. In particular embodiments, cells of libraries (or cells used to make libraries) can be modified to express a pro- and/or anti-viral factor. In particular embodiments, a pro- and/or anti-viral factor can be added to the environment of cells of libraries (or cells used to make libraries).

In certain examples, proviral factors include proteases (e.g., furin, trypsin, trypsin-like serine proteases, cathepsin L/D).

In particular embodiments, proviral factors of SARS-CoV2 include ACE2, CD147, AXL, HS, NRP1/2, SR-BI, ASGR1/KREMEN1, HMGB1, RAB7A, TMPRSS2/4/11, Furin, Cathepsin L, PIKfyve, TPC2, TMEM106B, SRPK1/2, VPS34, and SCAP.

In particular embodiments, proviral factors are viral proteins that are required to release viral particles (such as neuraminidase for influenza). In particular embodiments, proviral factors of influenza A virus include importin-α, importin-β, ANP32, epidermal growth factor recetpr (EGFR), receptor tyrosine kinases (RTKs), Rab GTPases, TMPRSS2 (transmembrane protease serine 2) and HAT (human airway trypsin-like protease), phosphatidylinositol 3-kinase (PI3K), HDAC6, dynein, dynactin and myosin II, PTBP1, NHP2L1, SNRP70, SF3B1, SF3A1, P14 and PRPF8, vacuolar-type ATPases, serine proteases, HSP90AA1, AMK2B, cellular RNA pol II, CLK1 (CDC-like kinase 1), CRM1, Golgi-specific brefeldin A-resistant guanine nucleotide exchange factor GBF1, JAK1, Raf/MEK/ERK pathway, and IKK/NFκB pathway.

In particular embodiments, proviral factors include IKKa, SREBPI, and ubiquitin-specific protease 7 (USP7/HAUSP).

In particular embodiments, a proviral factor for Ebola is NPC1.

In particular embodiments, proviral factors of HIV-1 include CD4, CCR5/CXCR4, retrograde Golgi transport proteins (Rab6 and Vps53) in viral entry, a karyopherin (TNPO3) in viral integration, the Mediator complex (Med28) in viral transcription, NFATc, and Rab11-FIP1C. For HIV Envelope, over-expressing Furin during viral production can be pro-viral.

Proviral drugs include those that can increase lentivirus production from cells (e.g., sodium butyrate, caffeine, etc).

Polycations can reduce negative charge repulsion between viral entry proteins and the cell membranes. Particular embodiments can use polybrene or DEAE dextran added during infection at a level and for a time that is non-toxic to the cells.

Amophotrericin B increases the infectivity of viruses pseudo-typed with SARS spike, but inhibits the infectivity of viruses pseudo-typed with HIV Envelope.

Antiviral factors or antiviral restriction factors are host cellular proteins that constitute a first line of defense, blocking viral replication and propagation.

In particular, embodiments, host antiviral factors of SARS-CoV2 include HD5, PSGL-1, Sialic acids, CH25H, LY6E, ZAP, and LARP1.

In particular embodiments, host antiviral factors of HIV-1 include APOBEC3G, SAMHD1, Tetherin/BST-2, TRIM5a, MX-2, SERINC3/5, IFITMs, Schlafen 11, and MARCH2/8.

In particular embodiments, antiviral factors of influenza virus include B4GALNT2, Viperin, PAl-1, BST-2, Cyclin D3, RIN2, TM9SF2, ZMPSTE24, IFITM2, IFITM3, MOV10, MxA, ISG15, TRIM32, TRIM22, ZAPL, CypA, PKR, ZAPS, Mx1, TRIM56, ISG20, PKP2, DDX21, and CypE.

Additional examples of antiviral factors include IFITM1 (Accession number: NM_003641), IFITM2 (ACCESSION NM_006435), IFITM3 (ACCESSION NM_021034.3, NR_049759.2 (non-coding)), ZMPSTE24 (ACCESSION NM_005857), CH25H (ACCESSION NM_003956), LY6E (Accession NM_002346.3, NM_001127213.2), NCOA7 (Accession NM_181782.5, NM_001122842.3, NM_001199619.2, NM_001199620.2, NM_001199621.2, NM_001199622.2, KC238672.1 (NCOA7-AS)); GILT (Accession: AF097362.1), CD74 (Accession NM_001025159.3, NM_004355.4, NM_001025158.3, NM_001364083.3, NM_001364084.3, NR_157074.3 (non-coding RNA)), ADAP2 (Accession NM_001346712.2, NM_018404.3, NM_001346714.2, NM_001346716.2, NR_144488.2 (non-coding)), and ZAP (Accession NM_020119.4, NM_024625.4, NM_001363491.2).

In particular embodiments, a library of 10⁴ to 10⁵ variants of a given protein is constructed and selection for function is imposed. Under modest selection pressure, variant frequencies are perturbed according to the function of each variant. Variants harboring beneficial mutations increase in frequency, whereas variants harboring deleterious mutations decrease in frequency. In particular embodiments, high throughput sequencing can measure the frequency of each variant during the selection experiment, and a functional score can be calculated from the change in frequency over the course of the experiment. In particular embodiments, the result is a large scale mutagenesis data set containing a functional score for each variant in the library. Fowler et al. (2014) Nature Protocols 9: 2267-2284.

In particular embodiments, the selection pressure is heat. Heat can include temperatures above 25° C., above 26° C., above 27° C., above 28° C., above 29° C., above 30° C., above 31° C., above 32° C., above 33° C., above 34° C., above 35° C., above 36° C., above 37° C., above 38° C., above 39° C., above 40° C., above 41° C., above 42° C., above 43° C., above 44° C., above 45° C., above 46° C., above 48° C., above 49° C., above 49° C., above 50° C., or more. In particular embodiments, heat can include temperatures from 28° C. to 70° C. In particular embodiments, heat can include temperatures from 30° C. to 65° C. In particular embodiments, heat can include temperatures above 30° C. In particular embodiments, the selection pressure is cold. Cold can include temperatures below 25° C., below 24° C., below 23° C., below 22° C., below 21° C., below 20° C., below 19° C., below 18° C., below 17° C., below 16° C., below 15° C., below 14° C., below 13° C., below 12° C., below 11° C., below 10° C., below 9° C., below 8° C., below 7° C., below 6° C., below 5° C., below 4° C., below 3° C., below 2° C., below 1° C., below 0° C., or lower. In particular embodiments, cold can include temperatures from 22° C. to 0° C. In particular embodiments, cold can include temperatures from 20° C. to 4° C. In particular embodiments, cold can include temperatures below 20° C. In particular embodiments, the selection pressure is low pH. Low pH can include pH of 6.9, 6.5, 6.0, 5.5, 5.0, 4.5, 4.0, 3.5, 3.0, 2.5, 2.0, or lower. In particular embodiments, low pH can be from pH of 6.8 to 2.0. In particular embodiments, low pH can be from pH of 6.5 to 3.0. In particular embodiments, low pH can include a pH below 6.5. In particular embodiments, the selection pressure is high pH. High pH can include pH of 7.5, 7.6, 7.7, 7.8, 7.9, 8.0, 8.5, 9.0, 9.5, 10.0, 10.5, 11.0, 11.5, 12.0, or higher. In particular embodiments, high pH can include a pH of 8.0 to 14.0. In particular embodiments, high pH can include a pH of 8.5 to 12.0. In particular embodiments, high pH can include a pH above 8.0. In particular embodiments, the selection pressure is a toxic agent. Toxic agents can include polar organic solvents (e.g., dimethylformamide), herbicides (e.g., glyphosate), pesticides (e.g., malathion, dichlorodiphenyltrichloroethane), salinity, ionizing radiation, and hormonally active phytochemicals (e.g., flavonoids, lignins and lignans, coumestans, or saponins).

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Exemplary Embodiments

1. A method of creating a mutational scanning library of variants of a viral protein including:
transfecting a population of cells with:
   a plurality of viral vectors, each viral vector including
      a functional 3′LTR,
      an inducible promoter operably linked to a nucleic acid encoding a bar code and a variant of a viral protein; and
      a constitutive promoter operably linked to a reporter and a selectable marker;
   a pseudo-typing expression plasmid; and helper plasmids,
   wherein viral vectors within the plurality have distinct bar codes and encoded variant viral proteins in relation to other viral vectors within the plurality, and wherein the transfecting results in production of pseudo-typed viruses;
infecting cells with the pseudo-typed viruses at a low multiplicity of infection (MOI); and selecting for infected cells
thereby creating the mutational scanning library of variants of the viral protein.

2. The method of embodiment 1, further including:
inducing expression of the variant of the viral protein in the infected cells; and
transfecting the infected cells with helper plasmids.

3. The method of embodiment 1 or 2, wherein the low MOI results in each infected cell being infected by only one pseudo-typed virus.

4. The method of any of embodiments 1-3, wherein the inducible promoter is the reverse tetracycline-controlled transactivator (rtTA) promoter.

5. The method of any of embodiments 1-4, wherein the selecting includes administering puromycin.

6. The method of any of embodiments 1-5, wherein the variants of the viral protein include viral entry protein variants.

7. The method of any of embodiments 1-6, wherein the variants of the viral protein are selected from severe acute respiratory syndrome coronavirus (SARS-CoV), SARS-CoV-2, Chikungunya, Ebola, Hendra, hepatitis B, hepatitis C, human immunodeficiency virus (HIV)-1, HIV-2, HIV, Env, simian immunodeficiency virus (SIV), influenza, Lassa, measles, Middle East respiratory syndrome coronavirus (MERS-CoV), Nipah, Rabies, or respiratory syncytial virus (RSV) viral proteins.

8. The method of any of embodiments 1-7, wherein the variants of the viral protein include variants of a viral entry protein selected from SARS-CoV-2 Spike (S), influenza hemagglutinin (HA), HIV envelope (Env), Chikungunya E1 Env, Chikungunya E2 Env, Ebola glycoprotein (EBOV GP), Hendra F glycoprotein, Hendra G glycoprotein, hepatitis B large (L), hepatitis B middle (M), hepatitis B small (S), hepatitis C glycoprotein E1, hepatitis C glycoprotein E2, Lassa virus envelope glycoprotein (LASV GP), measles hemagglutinin glycoprotein (H), measles fusion glycoprotein F0 (F), MERS-CoV Spike (S), Nipah fusion glycoprotein F0 (F), Nipah glycoprotein G, Rabies virus glycoprotein G (RABV G), RSV fusion glycoprotein F0 (F), or RSV glycoprotein G.

9. The method of embodiment 8, wherein the viral entry protein is the S protein of SARS-CoV-2 or HIV Env.

10. The method of any of embodiments 1-9, wherein the variants of the viral protein include viral Gag Pol variants.

11. The method of any of embodiments 1-10, wherein the variants of the viral protein include viral Tat variants.

12. The method of any of embodiments 1-11, wherein the variants of the viral protein include viral Rev variants.

13. The method of any of embodiments 1-12, wherein the viral vector includes a retroviral vector.

14. The method of embodiment 12, wherein the retroviral vector includes a lentiviral vector.

15. The method of any of embodiments 1-14, wherein the barcode includes 4 to 30 nucleotides.

16. The method of any of embodiments 1-15, wherein the barcode is located after the stop codon of the variant sequence.

17. The method of any of embodiments 1-16, wherein the population of cells includes 293T, HEK293T/17, HEK293F, HEK293S, HEK293SGH, EK293FTM, HEK293SGGD, GP2-293, HeLa, HeLa S3, HeLa B, HeLa T4, COS, COS-1, COS-6, COS-M6A, COS-7, A549, MDCK, HepG2, C2C12, THP-1, HUDEP-2, C8161, CCRF-CEM, MOLT, mIMCD-3, NHDF, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TI155, Jurkat, J45.01, LRMB, Bcl-1, BC-3, IC21, DLD2, Raw264.7, NRK, NRK-52E, MRC5, MEF, BS-C-1, monkey kidney epithelial, BALB/3T3 mouse embryo fibroblast, 3T3 Swiss, 3T3-L1, 132-d5 human fetal fibroblasts, 10.1 mouse fibroblasts, 3T3, 721, 9L, A2780, A2780ADR, A2780cis, A172, A20, A253, A431, A-549, ALC, E16, B35, BCP-1, BEAS-2E, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, Hepa1c1c7, HL-60, HMEC, HT-29, JY, K562, Ku812, KCL22, KG1, KYO1, LNCap, Ma-Mel 1-48, MC-38, MCF-7, MCF-10A, MDA-MB-231, MDA-MB-468, MDA-MB-435, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT2, RenCa, RIN-5F, RMA/RMAS, Saos-2, Sf-9, SkBr3, T2, T-47D, T84, THP1, U373, U87, U937, VCaP, Vero, WM39, WT-49, X63, YAC-1, or YAR cells.

18. The method of any of embodiments 1-17, wherein the transfected population of cells expresses or is exposed to a pro-viral factor.

19. The method of any of embodiments 1-18, wherein the infected cells express or are exposed to a pro-viral factor.

20. The method of any of embodiments 1-19, wherein the transfected population of cells expresses or is exposed to an anti-viral factor.

21. The method of any of embodiments 1-20, wherein the infected cells express or are exposed to an anti-viral factor.

22. A SARS-CoV-2 mutational scanning library as described herein.

23. Use of a mutational scanning library as described herein.

24. A method of identifying mutations in a viral protein that affect the sensitivity of the virus to a selection pressure using a mutational scanning library including barcoded cells encoding variant viral proteins, wherein the method includes:
obtaining the mutational scanning library including the barcoded cells encoding variant viral proteins, wherein at least 90% of the cells include a non-self-inactivating viral vector including a single homozygous barcoded variant nucleotide sequence from a set of homozygous barcoded variant nucleotide sequences in the library integrated into the storage cell's genome, wherein the set of homozygous barcoded variant nucleotide sequences collectively encode viral protein variants including at least 15 amino acid substitutions at at least 95% of amino acid positions of the viral protein;
transfecting the storage cells with plasmids including sequences encoding viral proteins for production of virions;
culturing the transfected storage cells to produce virions, wherein each virion includes a homozygous barcoded variant nucleotide sequence encoding the viral protein variant; exposing the virions to the selection pressure;
sequencing barcodes of variant nucleotide sequences from surviving virions; and linking sequenced barcodes to encoded viral protein variants to identify mutations in each surviving variant relative to a reference under the selection pressure, thereby identifying mutations in the viral protein that affect the sensitivity of a virus to the selection pressure.

25. The method of embodiment 24, wherein each viral protein variant is expressed.

26. The method of embodiment 24 or 25, wherein the reference is a counterpart viral protein of a wild-type virus, of a parental virus, or of a baseline clinical isolate.

27. The method of any of embodiments 24-26, wherein the selection pressure is a therapeutic compound.

28. The method of embodiment 27, wherein the therapeutic compound is undergoing pre-clinical development.

29. The method of embodiment 27 or 28, wherein the therapeutic compound is undergoing clinical development.

30. The method of any of embodiments 27-29, wherein the therapeutic compound includes viral entry and/or fusion inhibitors.

31. The method of any of embodiments 27-30, wherein the therapeutic compound is an antibody, or sera from humans or animals following infection or vaccination.

32. The method of embodiment 31, wherein the antibody is disclosed herein in relation to SAR-CoV-2 and/or selected from leronlimab (PRO 140), PRO 542, TNX-355 (ibalizumab), human monoclonal IgG1 anti-gp120 antibody b12, polyclonal caprine anti-HIV antibody PEHRG214, anti-HIV antibody PGT121, anti-HIV antibody 3BNC117, anti-RSV G protein monoclonal antibody clone 131-2G, anti-CXCR4 monoclonal antibody clone 12G5 12G5, anti-RSV F protein antibody MAB8582, anti-RSV F protein antibody MAB8581, anti-RSV F protein antibody MCA490, anti-RSV F protein antibody 104E5, anti-RSV F protein antibody 38F10, anti-RSV F protein antibody 14G3, anti-RSV F protein antibody 90D3, anti-RSV F protein antibody 56E11, anti-RSV F protein antibody 69F6, anti-Ebola virus glycoprotein (GP) monoclonal antibody c13C6, anti-Ebola virus glycoprotein (GP) monoclonal antibody c2G4, anti-Ebola virus glycoprotein (GP) monoclonal antibody c4G7, anti-Ebola virus glycoprotein (GP) monoclonal antibody c1H3, LCA60, REGN3051, REGN3048, anti-Lassa virus glycoprotein antibody 37.2D, anti-Lassa virus glycoprotein antibody 8.9F, anti-Lassa virus glycoprotein antibody 19.7E, anti-Lassa virus glycoprotein antibody 37.7H, anti-Lassa virus glycoprotein antibody 12.1F, and Hendra virus neutralizing antibody m102.4.

33. The method of any of embodiments 27-32, wherein the therapeutic compound includes a small molecule, a protein, a peptide, a polynucleotide, a polysaccharide, an oil, a solution, or a plant extract.

34. The method of any of embodiments 27-33, wherein the selection pressure is selected from heat, cold, low pH, high pH, and a toxic agent.

35. The method of any of embodiments 27-34, wherein the selection pressure is the ability of the virus to enter (i) a host cell of a species or (ii) a cell expressing a receptor protein of a species that is different from the species from which the cell was derived, wherein the ability is not dependent on presence of a functional unrelated viral entry protein.

36. The method of embodiment 35, wherein the species is human.

37. The method of embodiment 35 or 36, wherein the host cell is derived from human liver, human lung epithelia, or human lung.

38. An HIV Env mutational scanning library as described herein.

### Experimental Examples

### Example I—Design and Deep Mutational Scanning of the Full SARS-CoV-2 Spike

Design of lentiviral backbone and spike gene nucleotide sequence optimization. The structure of the lentiviral backbone is shown in FIG. 3B and the plasmid map of the lentivirus backbone containing BA.1 spike is at github.com/dms-vep/SARS-CoV-2_Omicron_BA.1_spike_DMS/blob/main/library_design/reference_sequences/3282_pH2rU3_F orlnd_Omicron_sinobiological_BA.1_B11529_Spiked21_T7_CMV_ZsGT2APurR.gb. Map for the Delta spike-containing backbone is at github.com/dms-vep/SARS-CoV-2_Delta_spike_DMS/blob/main/library_design/reference_sequences/pH2rU3_Forlnd_sinobiolog ical_617.2_Spiked21_CMV_ZsGT2APurR.gb. The vector is based on pHAGE2 lentiviral backbone in which the 3′ LTR sequence was repaired, which allowed for the re-rescue of the pseudovirus from the cells in which lentiviral backbones have been integrated. The lentiviral backbone is non-replicative unless helper plasmids (Gag/Pol (NR-52517), Tat1b (NR-52518), and Rev1b (NR-52519)) are also transfected into the cells containing this backbone. Expression of the spike gene in the lentivirus backbone is driven both by inducible TRE3G promoter and by Tat1b. TRE3G promoter is activated by the addition of doxycycline in the presence of the reverse tetracycline transactivator (rtTA), which is endogenously expressed in HEK-293T-rtTA cells. The spike gene has been codon optimized and lacks 21 amino acids in its cytoplasmic tail. The cytoplasmic tail deletion has been previously shown to significantly increase pseudovirus titers (Havranek et al., 2020, Viruses 12, 1465). For spike sequence codon optimization, a large panel of optimized sequences was tested and it was found that virus titers can vary between codon optimizations by as much as 100-fold. Of the tested codon optimizations, the sequence optimized spike from SinoBiological (VG40609-UT) gave by far the best virus titers; therefore all variant sequences were based on the original SinoBiological optimization. In addition to the inducible promoter and spike gene, the backbone also has a CMV promoter that drives expression of the ZsGreen gene linked by a T2A linker to the puromycin resistance gene. ZsGreen is used as a reporter gene to detect pseudovirus infection and the puromycin resistance gene is used as a selection marker for cells with successfully integrated lentiviral backbones.

Design of spike mutations to include in BA.1 and Delta full spike deep mutational scanning libraries. A library was created with mutations that would result in mostly functional spike proteins and would be important for the antigenic evolution of spike. To this end, for BA.1 and Delta deep mutational scanning libraries, the following types of mutations were included: (1) mutations (nonsynonymous changes and deletions) observed in spike sequences deposited on the GISAID database, (2) mutations that reoccur in spike phylogeny independently multiple times, (3) all possible amino acid changes at sites in spike that show positive selection. Specifically for the BA.1 library, all possible amino acid changes were included for sites that are mutated in the BA.1 spike relative to Wuhan-Hu-1.

The following criteria were used to select the above described mutations for the BA.1 library: nonsynonymous mutations need to be present in the GISAID database >16 times, deletions need to occur in the NTD and be observed on the GISAID database >300 times, nonsynonymous mutations need to reoccur on spike phylogenetic tree independently at least 21 times. To get all spike mutations observed in GISAID deposited sequences a CoVsurver curated spike amino acid frequency table (with sequences deposited up to Jan. 31, 2022) (Khare et al., 2021, GISAID's Role in Pandemic Response. China CDC Wkly. 3, 1049-1051) was used. To get independently recurring spike mutation counts pre-built SARS-CoV-2 phylogenies from UShER (Turakhia et al., 2021, Nat. Genet. 53, 809-816) were used. Information on sites in spike undergoing positive selection was taken from taken from table here raw.githubusercontent.com/spond/SARS-CoV-2-variation/master/windowed-sites-fel-2021-07.csv which was built using methods described in Maher et al. (2022, Sci. Transl. Med. 14, eabk3445). The full list of mutations included in the BA.1 library can be found at github.com/dms-vep/SARS-CoV-2_Omicron_BA.1_spike_DMS/blob/main/library_design/results/aggregated_mutations.csv.

The following criteria were used to select the above described mutations for the Delta library: nonsynonymous mutations and deletions need to be observed on the GISAID database more than once and nonsynonymous mutations need to reoccur on spike phylogenetic tree independently more than 7 times. To get all spike mutations observed in GISAID deposited sequences all spike sequences deposited on GISAID up to Jul. 26, 2021 were aligned and mutation frequency counts were extracted. Independently recurring spike mutations and positively selected sites were identified as described for BA.1 library above. The full list of mutations included in the Delta library can be found at github.com/dms-vep/SARS-CoV-2_Delta_spike_DMS/blob/main/library_design/results/aggregated_mutations.csv.

Design of primers for BA.1 and Delta spike mutagenesis. For each set of mutations described in the section above separate primer pools were designed: (1) a pool of primers for observed mutations, (2) a pool of primers for recurrent mutations, (3) a pool of primers for positive selection site mutations, and (4) a pool of primers that would cover changes at multiple positive selection sites if those positive selection sites are close enough to each other so that the primers in the pool (3) would overlap. For the BA.1 library primers were also designed that would introduce multiple amino acid deletions at recurrent deletion regions described in (McCarthy et al., 2021, Science 371, 1139-1142) and included them in the observed mutation primer pool. Also for the BA.1 library, the set of primers that cover all possible amino acid changes at the sites already mutated in BA.1 was pooled with the positive selection site primer pool.

All primer pools were ordered from Integrated DNA Technologies as oPools. Scripts for designing the BA.1 library primer pools and the resulting oPools that were ordered can be found at github.com/dms-vep/SARS-CoV-2_Omicron_BA.1_spike_DMS/tree/main/library_design. Scripts for designing the Delta library primer pools and the resulting oPools that were ordered can be found at github.com/dms-vep/SARS-CoV-2_Delta_spike_DMS/tree/main/library_design.

Design of full-spike deep mutational scanning plasmid libraries. Making of the plasmid libraries for deep mutational scanning required the following three steps (1) mutagenesis of the spike gene, (2) barcoding of the mutagenized spike sequence, and (3) cloning of the mutagenized and barcoded spike into the lentiviral backbone-carrying plasmid.

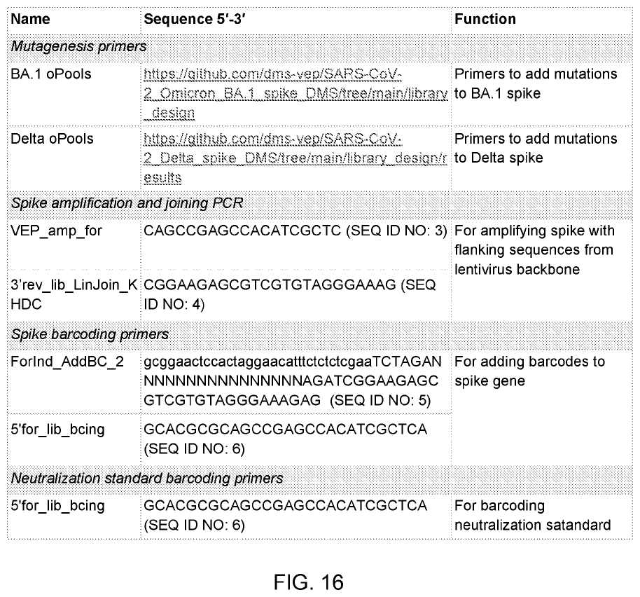

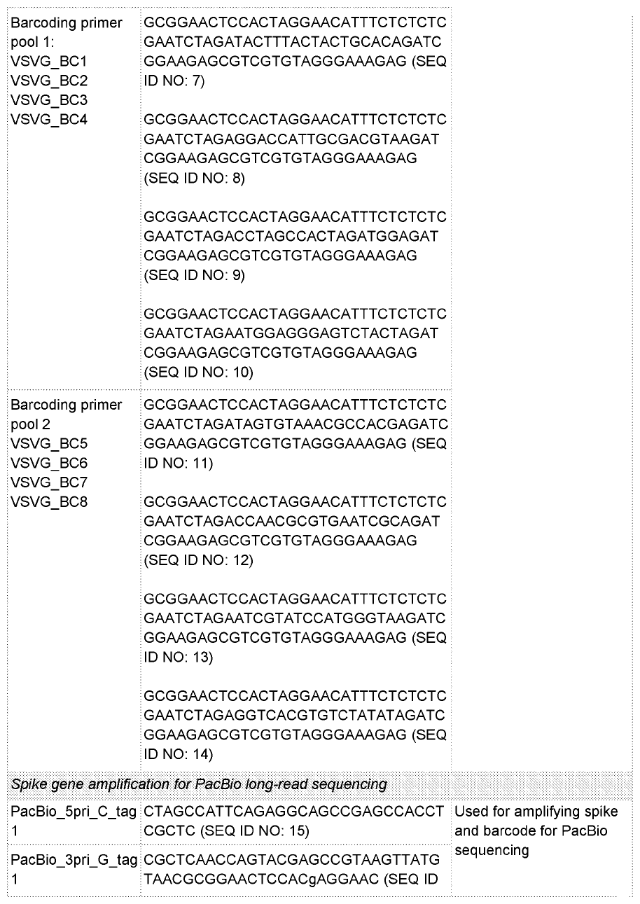

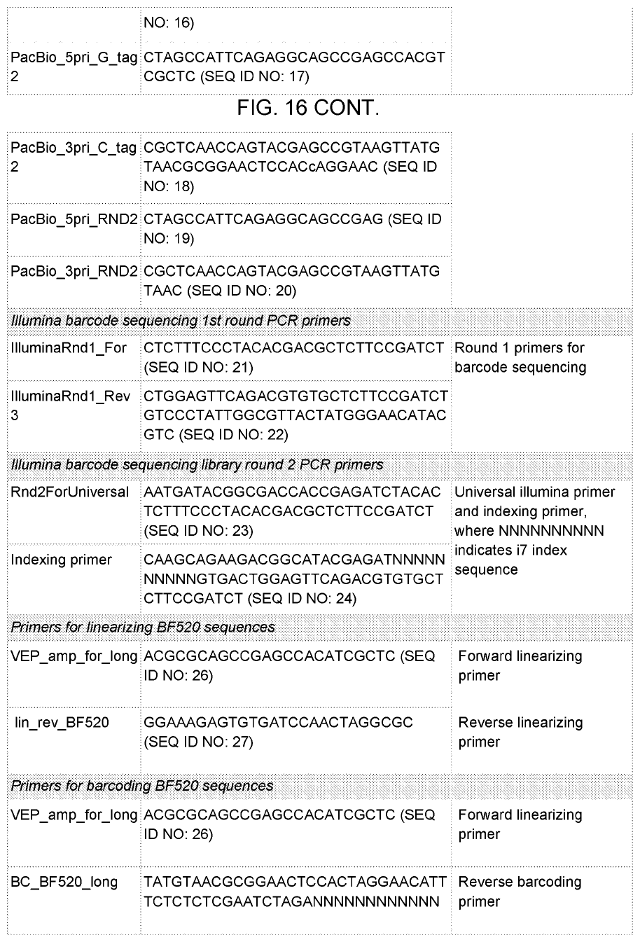

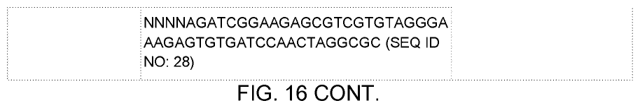

Spike mutagenesis was carried out by first amplifying BA.1 or Delta spike gene sequence from a plasmid carrying lentiviral backbone with a codon optimized spike sequence (see section ‘Design of lentiviral backbone and spike gene nucleotide sequence optimization’ for plasmid maps). The spike sequence was amplified using ‘Spike amplification’ primers from FIG. 16 with the following PCR conditions: 1.5 μl of 10 μM forward primer, 1.5 μl of 10 μM reverse primer, 10 ng of amplified spike gene template, 25 μl of KOD polymerase (KOD Hot Start Master Mix, Sigma-Aldrich, Cat. No. 71842), and water for the final volume of 50 μl. PCR cycling conditions were as follows: 95° C. for 2 min; 95° C. for 20 s; 62° C. for 15 s; 70° C. for 2 min (return to step 2 for another 19× cycles); Hold at 4° C. Amplified spike sequence was first gel-purified using NucleoSpin Gel and PCR Clean-up kit (Takara, Cat. No. 740609.5) and then further purified using Ampure XP beads (Beckman Coulter, Cat. No. A63881) at 1:2.6 sample to bead ratio.

Next, the purified spike template was used in mutagenesis PCR using the protocol described previously in (Bloom, 2014, Phylogenetic Fit. Mol. Biol. Evol. 31, 1956-1978) with a few modifications. Primers for mutagenesis PCR were pooled at 1:2:2:0.2 molar ratio between observed primer pool:recurrent primer pool:positive selection primer pool:paired positive selection primer pool. The pooling ratios are determined by the fact that recurrent and positively selected sites may be more antigenically and structurally important for spike. Two independent mutagenesis reactions were performed for each spike creating two independent biological library replicates (which means that they will have a unique set of barcodes and a unique set of mutation combinations in spike). For BA.1 libraries two rounds of mutagenesis were performed with the first round including 8 mutagenic PCR cycles followed by the second round of 10 mutagenic PCR cycles. For Delta libraries one biological replicate including a single round of 10 mutagenic PCR cycles and the second biological replicate included one round of 8 and another round of 10 mutagenic PCR cycles. Between each mutagenic PCR round, 20 cycles of joining PCR were performed and the mutagenized spike templates were gel and Ampure XP purified.

After the spike sequence was mutagenized a barcoding PCR that appended a random 16 nucleotide barcode sequence downstream of the spike gene stop codon was performed. 16 nucleotide barcodes were chosen as this allows for a total of 416 unique barcoded variants, which is a much greater diversity of barcodes than the final size of the disclosed deep mutational scanning plasmid libraries and therefore limits potential barcode duplications. For barcoding ‘Spike barcoding’ primers from FIG. 16 were used with the following PCR conditions 1.5 μl of 10 μM forward primer, 1.5 μl of 10 μM reverse primer, 30 ng of the mutagenised spike gene template, 25 μl of KOD polymerase, and water for the final volume of 50 μl. PCR cycling conditions were as follows: 95° C., 2 min; 95° C., 20 s; 70° C., 1 s; 55.5° C., 20 s, cooling at 0.5° C./s; 70° C., 2 min (return to step 2 for another 9× cycles); 4° C. hold. The mutagenized and barcoded spike was then cloned into lentiviral backbone-containing plasmid. First, a lentiviral backbone containing plasmid using MluI and XbaI restriction sites was digested. The map of the plasmid used for vector digestion can be found at github.com/dms-vep/SARS-CoV-2_Omicron_BA.1_spike_DMS_mAbs/blob/main/library_design/reference_sequences/other_plasmid_maps_for_library_design/3137_pH2rU3_Forlnd_mCherry_CMV_Zs GT2APurR.gb.

Digested vector was gel and Ampure XP purified. A 1:3 insert-to-vector ratio was used in a 1 hour Hifi assembly reaction using NEBuilder HiFi DNA Assembly kit (NEB, Cat. No. E2621). After the HiFi assembly, Ampure XP purified the reaction and eluted it in 20 μl of water (note that elution in water as opposed to elution buffer enhances the subsequent electroporation efficiency). 1 μl of the purified HiFi product was used to transform 20 μl of 10-beta electrocompetent E. coli cells (NEB, C3020K). 10 electroporation reactions were performed to get a final count of >2 million CFUs per library and plated transformed cells out on LB+ ampicillin plates. The plasmid library was made from a much greater number of CFUs than the number of variants in the disclosed final virus libraries to minimize barcode duplication, as explained in the next section. 16 hours after transformation bacterial colonies were scraped using liquid LB+ ampicillin and plasmid stocks were prepared using QIAGEN HiSpeed Plasmid Maxi Kit (Cat. No. 12662). The final structure of the lentiviral genome with a mutagenized spike cloned into it is shown in FIG. 3B.

Production of cell-stored spike deep mutational scanning libraries. Production of cell-stored deep mutational scanning libraries required the following steps: (1) production of VSV G pseudotyped lentivirus, (2) infection of rtTA-expressing cells with VSV G pseudotyped virus, and (3) selection for transduced cells. These steps are illustrated in FIG. 3C.

To generate VSV G pseudotyped virus for each library 0.5 M HEK-293T cells per eight wells were plated of two 6-well tissue culture dishes. The aim was to produce VSV G pseudotyped virus stocks that have a greater number of infectious particles than the number of colonies scraped for plasmid libraries in order to not introduce any bottleneck on barcodes at this stage. The next day 0.25 μg of each helper plasmid (Gag/Pol, Tat1b, and Rev1b), 0.25 μg of VSV G expression plasmid github.com/jbloomlab/SARS-CoV-2-BA.1_Spike_DMS_validations/blob/main/plasmid_maps/29_HDM_VSV_G.gb) and 1 μg of mutagenized and barcoded spike containing lentiviral vector (described in the section above) were transfected. Transfections were done using BioT reagent (Bioland Scientific, Cat. No. B01-02) according to the manufacturer's instructions. 48 hours post-transfection supernatants from each well were pooled, filtered through a surfactant-free cellulose acetate 0.45 μm syringe filter (Corning, Cat. No. 431220), and stored at −80° C. VSV G pseudotyped viruses were titrated as described in (Crawford et al., 2020, Viruses 12, 513).

Next HEK-293T-rtTA cells were infected with the generated VSV G pseudotyped virus. The number of infectious virus units used in these infections allowed for the bottlenecking of the library size at the desired final variant number. BA.1 libraries were bottlenecked at 100,000 variants and Delta libraries were bottlenecked at 50,000 variants. Notably, a substantially lower number of variants to infect cells was used compared to the possible diversity of variants in the disclosed plasmid libraries. This allows for limiting any potential duplication of barcodes between different variants due to recombination in the lentivirus genome, which would be (the number of infectious viruses used to make the library)/(number of colonies used to make the plasmid library). Note for BA.1 libraries Lib-1 and Lib-2 originate from the same mutagenized lentiviral backbone plasmid stock but independent VSV G virus infections and Lib-3 originates from independent mutagenized plasmid library stock. For Delta libraries Lib-1 and Lib-2 are both from independent mutagenised spike plasmid stocks. Infections were performed at MOI <0.01 (in order to ensure that only a single spike variant is integrated in each cell), which was verified 48 hours after infection using fluorescence-activated cell sorting by detecting ZsGreen expression from the lentiviral backbone. After MOI was verified, cells were expanded for another 48 hours, and then started puromycin selections to select for cells with successfully integrated lentivirus genomes. The selection was done using 0.75 μg/ml of puromycin with a fresh change of puromycin-containing D10 (see ‘Cell lines’ section below) every 48 hours. Selections were terminated when visual inspection using a fluorescent microscope indicated that all cells express ZsGreen (6-8 days). After puromycin selection was finished cells were expanded for another 48 hours in fresh D10 and frozen cell aliquots in tetracycline-free FBS ((Gemini Bio, Cat. No. 100-800) containing 10% DMSO. Frozen cell aliquots were stored in liquid nitrogen long-term.

Generation of spike and VSV G-pseudotyped viruses from cell-stored spike deep mutational scanning libraries. To generate spike pseudotyped viruses from cell-stored deep mutational scanning libraries 100 million library-containing cells were plated per 5-layer flask (Corning Falcon 875 cm² Rectangular Straight Neck Cell Culture Multi-Flask, Cat. No. 353144) in 150 ml of D10 without phenol red supplemented with 1 μg/ml for doxycycline (which allows to induce spike expression ahead of pseudovirus production). 24 hours after plating cells were transfected with 50 μg of each helper plasmid (Gag/Pol, Tat1b, Rev1b) using BioT reagent according to the manufacturer's instructions. 48 hours post transfection cell supernatant was collected and filtered through a 0.45 μm SFCA Nalgene 500 mL Rapid-Flow filter unit (Cat. No. 09-740-44B). Filtered supernatant was then concentrated by spinning at 4° C. 3000 rcf for 30 min using Pierce Protein Concentrator (ThermoFisher, 88537). Virus aliquots were stored long-term at −80° C.

To generate VSV G pseudotyped viruses (for functional selection and long-read PacBio sequencing) from cell-stored deep mutational scanning libraries 60 million library-containing cells were plated per 3-layer flask (Corning Falcon 525 cm² Rectangular Straight Neck Cell Culture Multi-Flask, 353143) in 90 ml of D10 without phenol red (doxycycline was not added in this case). 24 hours after plating cells were transfected with 30 μg of each of the helper plasmid (Gag/Pol, Tat1b, Rev1b) and 18.75 μg of VSV G expression plasmid using BioT reagent according to the manufacturer's instructions. 32-36 hours post transfection cell culture supernatant was collected and filtered through a 0.45 μm SFCA Nalgene filter unit. Filtered supernatant was then concentrated by spinning at 4° C. 3000 rcf for 30 min using Pierce Protein Concentrator. Virus aliquots were stored long-term at −80° C.

Long-read PacBio sequencing of barcoded spike variants in deep mutational scanning libraries. Long-read PacBio sequencing was used to acquire reads spanning the spike and the random 16 nucleotide barcode sequences. To prepare amplicons for PacBio sequencing 1 million HEK-293T cells were infected with 30 million VSV G pseudotyped lentiviruses carrying the deep mutational scanning libraries. This number of viruses is significantly greater than the expected number of variants in the library, which achieves high variant coverage, avoids bottleneck of barcode diversity, and corrects for any potential PCR or sequencing errors. 12-15 hours after infection cells were trypsinized, washed with PBS and non-integrated lentiviral genomes were recovered using QIAprep Spin Miniprep Kit (Cat. No. 27106X4) (Dingens et al., 2018, PLOS Pathog. 14, e1007159; Haddox et al., 2016, PLOS Pathog. 12, e1006114). Non-integrated viral genomes were used as the disclosed sequencing templates because they are the more abundant forms of the lentiviral genome than the integrated proviruses (Chun et al., 1997, Nature 387, 183-188; Pang et al., 1990, Nature 343, 85-89; Sharkey et al., 2000, Nat. Med. 6, 76-81; Van Maele et al., 2003, J. Virol. 77, 4685-4694). Elution volume for the miniprep was adjusted to 144 μl. Next two rounds of PCR were performed to amplify the region in the lentivirus genome spanning the spike and the random 16 nucleotide barcode. In the first round of PCR primers were used containing single nucleotide tags, which allows for later detection of strand exchange that may occur during PCR amplification. To limit strand exchange during PCR (which would disrupt barcode/spike variant linkage) the number of PCR cycles performed was minimized and multiple PCR reactions per sample were performed (Liu et al., 2014, PLoS ONE 9, e106658; Omelina et al., 2019, BMC Genomics 20, 536). Each sample was split into eight PCR reactions, four of which use ‘tag_1’ forward and reverse primers and four of which use ‘tag_2’ forward and reverse primers from the ‘Spike gene amplification for PacBio long-read sequencing’ primer set in FIG. 16. PCR reaction conditions were as follows: 1 μl of forward primer, 1 μl of reverse primer, 20 μl of KOD, and 18 μl of sample. PCR cycling conditions for round 1 PCR were as follows: 95° C. for 2 min; 95° C. for 20 s; 70° C. for 1 s; 60° C. for 10 s (ramp 0.5° C./s); 70° C. for 2.5 min (go to 2 for another 7 cycles); 70° C. for 5 min; 4° C. hold.

After the first PCR round, all reactions were pooled for each sample and purified using Ampure XP beads with 1:0.8 beads to sample ratio and the PCR product was eluted in 84 μl of elution buffer. Eluted PCR product was divided into four PCR tubes and the second round of PCR was performed using ‘RND2’ forward and reverse primers from the ‘Spike gene amplification for PacBio long-read sequencing’ primer set in FIG. 16. PCR reaction conditions were as follows: 2 μl of forward primer, 2 μl of reverse primer, 25 μl of KOD, and 21 μl of purified sample. PCR cycling conditions were the same as for the round 1 PCR for a total of 10 PCR cycles. PCR reactions for each sample were pooled, purified using Ampure XP beads with 1:0.8 beads to sample ratio, and eluted in 27 μl of elution buffer. Barcodes were attached to each sample using sample SMRTbell prep kit 3.0 before multiplexing. Multiplexed SMRTbell libraries were then bound to polymerase using Sequel II Binding Kit 3.2 and sequenced with PacBio Sequel IIe sequencer with a 20-hour movie collection time.

Antibody escape mapping using full spike deep mutational scanning libraries. For antibody escape mapping between 4-15 times more infectious virions than the estimated total number of barcodes in a deep mutational scanning library were used. Using significantly more infectious virions relative to the number of variants per library avoids bottlenecking by having multiple copies of each variant. Several fold more lentiviral genomes per selection experiment were expected compared to the number of infectious units used because the non-integrated viral genomes were recovered for sequencing, which are more abundant than integrated proviral DNA (Chun et al., 1997, Nature 387, 183-188; Pang et al., 1990, Nature 343, 85-89; Sharkey et al., 2000, Nat. Med. 6, 76-81; Van Maele et al., 2003, J. Virol. 77, 4685-4694) on which the disclosed library virus titers are based. For each antibody escape mapping experiment a master mix was made of library spike-pseudotyped virus mixed with VSV G pseudotyped neutralization standard (described below). Neutralization standard was added at 1-2% of the total virus titer used in the experiment. Virus master mix was then aliquoted into Eppendorf tubes to which either different mounts of antibody or no antibody was added. For Ly-CoV1404, CC9.104, and CC67.105 antibodies selection experiments were performed at 3 concentrations, starting with IC9 concentration predetermined using standard pseudovirus neutralization assay and then increasing this concentration 4 fold and 16 fold. Starting with IC9 concentration because around 1% of the library is expected to be able to escape antibody selection. Additional concentrations were used as it helps to cover a greater dynamic concentration range in cases where the exact IC9 value is difficult to determine. Also, the use of multiple concentrations enables more precise mutation-escape predictions by the biophysical model used to decompose single-mutation effects (Yu et al., 2022, bioRxiv 2022.09.17.508366). For Ly-CoV1404 starting concentration was 0.654 μg/ml, for CC9.104-68 μg/ml, for CC67.105-52.5 μg/ml. For the REGN10933 it was started at IC_{99.5} at 0.146 μg/ml and also increased that concentration by 4 fold and 16 fold. For the NTD 5-7 antibody, which does not fully neutralize the virus, it was started with >IC₉₆ concentration at 150 μg/ml and then increased that concentration by 2 fold. Virus was mixed with the antibody by inverting tubes several times, spun down at 300 g, and incubated at 37° C. for 1 h. After incubation virus and antibody mix or no antibody control were used to infect 0.5 million target cells, which were plated a day before in D10 supplemented with 2.5 μg/ml of amphotericin B (Sigma, Cat. No. A2942) (which increases viral titers as shown in FIG. 4B). The target cell line for different antibodies is determined by whether an antibody is able to neutralize pseudovirus on that cell line. As previously described in Farrell et al., (2022, Viruses 14, 2061), non-ACE2 competing antibodies do not fully neutralize pseudovirus on ACE2 overexpressing cells. While testing antibodies for the current example, also it was noticed that some S2-targeting antibodies are also not affected by ACE2 overexpression. Therefore, for Ly-CoV1404, CC9.104, and CC67.105 antibodies HEK-293T-ACE2 were used as target cells but for NTD-targeting 5-7 antibody HEK-293T-ACE2-medium cells were used. For REGN10933 HEK-293T-ACE2-TMPRSS2 were used as target cells because TMPRSS2 overexpression increases Delta pseudovirus titers. 12-15 hours after infection cells were trypsinized, washed with PBS and non-integrated lentiviral genomes were recovered using QIAprep Spin Miniprep Kit and eluted in 21 μl of Qiagen elution buffer. Barcode reads for each sample were then prepared for Illumina sequencing using a method described in the ‘Barcode amplicon preparation for Illumina sequencing’ section below.

Functional selections using full spike deep mutational scanning libraries. To perform functional spike selections 1 million HEK-293T-ACE2 cells were infected with 1-2 million of the spike or VSV G pseudotyped viruses produced from deep mutational scanning library carrying cells (described earlier). As for antibody selections, the amount of virus used is greater than the number of variants in each library which limits potential bottlenecking of the library barcodes. 12-15 hours after infection cells were trypsinized, washed with PBS and non-integrated lentiviral genomes were recovered using QIAprep Spin Miniprep Kit. Barcode reads for each sample were then prepared for Illumina sequencing using methods described in ‘Barcode amplicon preparation for Illumina sequencing’ section below.

Barcode amplicon preparation for Illumina sequencing. To prepare barcode reads for Illumina sequencing two rounds of PCR were performed. In the first round of PCR primers that align to Illumina Truseq Read 1 primer site located directly upstream of the barcode in the lentiviral backbone and a primer annealing downstream of the barcode containing an overhand with Illumina Truseq Read 2 sequence (see ‘Illumina barcode sequencing 1st round PCR primers’ in FIG. 16) were used. Conditions for the first round PCR were as follows: 1 μl of 10 uM forward primer, 1 μl of 10 uM reverse primer, 26 μl of KOD, and 24 μl of minipreped sample DNA. PCR cycling conditions for round 1 PCR were as follows: 95° C. for 2 min; 95° C. for 20 s; 70° C. for 1 s; 58° C. for 10 s, cooling at 0.5° C. per s; 70° C. 20 s (return to step 2 for another 27 cycles); 4° C. hold.

PCR reactions were purified with Ampure XP beads using a 1:3 sample to beads ratio and eluted in 37 μl of Qiagen elution buffer. Second round of PCR used primers primer annealing to the Illumina Truseq Read 1 primer site with P5 Illumina adapter overhang and reverse primers from the PerkinElmer NextFlex DNA Barcode adaptor set, which anneal to Truseq Read 2 site and contain P7 Illumina adapter and i7 sample index. Conditions for the second round PCR were as follows: 1.5 μl of 10 uM universal primer, 1.5 μl of 10 uM indexing primer, 25 μl of KOD, and 20 ng of first round PCR product. PCR cycling conditions were the same as the first round PCR for a total of 20 cycles. After the second PCR round, all samples were pooled at desired ratios and gel and Ampure XP bead purified. Barcode amplicons were sequenced using NextSeq 2000 with either P2 or P3 reagent kits.

Production of barcoded neutralization standard. To make the neutralization standard that was added to the disclosed deep mutational scanning libraries the same general barcoding approach as described above for the deep mutational scanning plasmid library generation was used with a few important differences. The lentiviral backbone used for neutralization standard includes TRE3G inducible mCherry protein and CMV promoter driven ZsGreen. The plasmid map of the template backbone is at github.com/dms-vep/SARS-CoV-2_Omicron_BA.1_spike_DMS_mAbs/blob/main/library_design/reference_sequences/other_plasmid_maps_for_library_design/2871_pH2rU3_Forlnd_mCherry_CMV_ZsG_NoBC_cloningvector.gb. Note this backbone does not encode any viral glycoproteins and to rescue VSV G pseudotyped virus VSV G expression plasmid in trans was provided. mCherry plasmid was amplified from the lentiviral template and barcoded it in two independent PCR reactions using 2 sets of primers containing 4 unique barcodes (see ‘Neutralization standard barcoding primers’ in FIG. 16). Importantly, the unique barcodes were balanced in a way that there's a unique nucleotide at each position of the 16-nucleotide barcode between each of the four barcoding primers in a PCR reaction. Furthermore, the 8 barcoding primers are unique to the neutralization standard and are not present in any of the disclosed deep mutational scanning libraries. The PCR for barcoding was done the same way as described for deep mutational scanning plasmid library production and both PCR reactions were pooled together before Hlfi assembly into the lentiviral backbone. Barcoded lentiviral backbone was then used to rescue VSV G pseudotyped lentiviruses that were then used to infect HEK-293T-rtTA cells at low MOI. Successfully transduced HEK-293T-rtTA were then selected by flow-activated fluorescence sorting and expanded. VSV G pseudotyped neutralization standard was generated by transfecting helper plasmids and VSV G expression plasmid in the same way as described for deep mutational scanning library virus rescues. Note, that the neutralization standard generated from the integrated cells was used as opposed to the original transfection in order to prevent any potential lentiviral backbone-containing plasmid contamination of the virus stocks that can occur when viruses are produced from transfections.

Validation of deep mutational scanning by pseudovirus titration and neutralization. Spike genes carrying desired mutations were cloned by performing PCR reactions with partially overlapping desired mutation-containing primers followed by HiFi assembly. HDM_omicron_B11529_IDTDNA plasmid was used as the template for PCR. The map of the plasmid can be found at github.com/jbloomlab/SARS-CoV-2-BA.1_Spike_DMS_validations/blob/main/plasmid_maps/3277_HDM_omicron_B11529_IDTDNA.gb. All plasmid sequences were verified using full plasmid sequencing by Primodium. Mutated spike plasmids or VSV G expression plasmids were then used to generate and titrate pseudoviruses as described in (Crawford et al., 2020, Viruses 12, 513) except that the backbone used for virus generation was pHAGE6_Luciferase_IRES_ZsGreen and which also only required Gag/Pol helper plasmid for virus rescues. Note, for the spike variants cloned to validate functional selections three replicate virus rescues were performed for each variant and each rescue was done using an independent plasmid preparation for that spike variant.

BA.1 spike variants rescued for functional selection validation were titrated on HEK-293T-ACE2 and Delta spike variants were titrated on HEK-293T-ACE2-TMPRSS2 cells. Duplicate serial dilutions were performed using supernatants collected from the virus rescues and measured luciferase expression at each dilution using Bright-Glo Luciferase Assay System (Promega, E2610). Virus titers were calculated as relative light units (RLU) per μl for each dilution and taking the average RLU/μl values across dilutions within a linear range. For spike variants used to validate antibody escape experiments virus titration was performed in the same way using the same target cells as the neutralization assays were performed in (see below).

For pseudovirus neutralization 12.5 thousand target cells were plated into poly-L-lysine coated, black-walled, 96-well plates (Greiner 655930) in D10 supplemented with 2.5 μg/ml of amphotericin B. For neutralization assays using Ly-CoV1404, CC9.104, or CC67.105 antibodies HEK-293T-ACE2 were used as target cells, for REGN10933 HEK-293T-ACE2-TMPRSS2 were used as target cells, and for NTD 5-7 mAb HEK-293T-ACE2-medium were used as target cells. The use of different cell lines for each antibody is determined by the ability of that antibody to neutralize the virus on that cell line as described previously. Next day replicate serial dilutions were prepared for each antibody. The starting concentration for each antibody was as follows: Ly-CoV1404—4 μg/ml, CC9.104 and CC67.105—300 μg/ml, 5-7—96 μg/ml, REGN10933—6 μg/ml. Serial dilutions were then mixed with pseudovirus and incubated for 1 h at 37° C. After incubation, the virus-antibody mix was transferred onto the target cells. 48-55 h after infection Bright-Glo Luciferase Assay System (Promega, E2610) was used to measure luciferase activity. Fraction infectivity for each antibody dilution was calculated by subtracting background readings and dividing RLU values in the presence of antibody by RLU values in the absence of it. Neutralization curves were then plotted by fitting a Hill curve to fraction infectivity values using neutcurve software (jbloomlab.github.io/neutcurve/, version 0.5.7). Neutcurve package was also used to extract target ICx values from the fitted neutralization curves.

Code for plotting virus titers and neutralization curves from this paper can be found at github.com/jbloomlab/SARS-CoV-2-BA.1_Spike_DMS_validations.

Cell lines. HEK-293T were acquired from ATCC (CRL3216), HEK-293T-ACE2 cells are described in (Crawford et al., 2020, Viruses 12, 513), generation and characterization of HEK-293T-ACE2-medium cells is described in (Farrell et al., 2022, Viruses 14, 2061) (referred to ‘medium’ cells in the reference), generation of HEK-293T-rtTA cells is described below. All cells were grown in D10 media (Dulbecco's Modified Eagle Medium with 10% heat-inactivated fetal bovine serum, 2 mM 1-glutamine, 100 U/mL penicillin, and 100 μg/mL streptomycin). For antibody selection experiments D10 was made with phenol-free DMEM (Corning DMEM With 4.5 g/L Glucose, Sodium Pyruvate; Without L-Glutamine, Phenol Red from Fisher, Cat. No. MT17205CV). For experiments with HEK-293T-rtTA cells, D10 was made with tetracycline-free FBS (Gemini Bio, Cat. No. 100-800) to avoid any expression of spike unless doxycycline is added.

To produce HEK-293T-rtTA expressing cells (used for storing deep mutational scanning libraries and required for TRE3G promoter activation) VSV G pseudotyped lentivirus were first generated carrying rtTA gene. To produce this virus 0.5 million HEK-293T cells were transfected with 0.25 μg of each helper plasmid (Gag/Pol, Tat1b, Rev1b), 0.25 μg of VSV G expression plasmid and 1 μg of lentiviral backbone carrying plasmid into which rtTA has been cloned (plasmid map github.com/dms-vep/SARS-CoV-2_Omicron_BA.1_spike_DMS_mAbs/blob/main/library_design/reference_sequences/other plasmid_maps_for_library_design/3137_pH2r U3_Forlnd_mCherry_CMV_ZsGT2APurR.gb). 48 hours after transfection virus-containing cell supernatant was collected and filtered through a surfactant-free cellulose acetate 0.45 μm syringe filter. 5 μl of the virus was used to infect 0.5 million low passage HEK-293T cells. 48 hours post infection single cell clones were sorted into a 96-well plate using BD Aria II cell sorter with 610/20 filter in PE-Texas Red channel. Single clones were expanded and tested for the ability to produce high virus titers. High virus titers are essential for performing deep mutational scanning experiments on a practical experimental scale and it was found that individual cell clones can vary significantly in the virus titers they can produce. The clonal cell population producing the highest virus titers was selected for expansion, frozen down in 10% DMSO and 20% FBS D10 media, and stored long-term in a liquid nitrogen freezer.

Antibodies. Ly-CoV1404 antibody was cloned and produced by GensScript. Variable domain sequences were taken from previously published antibody structure (Westendorf et al., 2022, bioRxiv 2021.04.30.442182). Ly-CoV1404 variable regions were cloned with IgG1 heavy chain and human IgL2 constant regions, expressed in mammalian cells and purified using IgG-binding columns.

Computational Analysis. Overview of data analysis pipeline. To analyze the deep mutational scanning data, a modular analysis pipeline was created. At the core of this pipeline is a set of common steps that are expected to be shared across analysis of many different datasets. This set of common steps was implemented in a standalone GitHub repository named dms-vep-pipeline which is publicly available at github.com/dms-vep/dms-vep-pipeline and is designed to be included in project-specific analyses as a git submodule. The dms-vep-pipeline includes a series of Snakemake (Molder et al., 2021, F1000Research 10:33) rules that run Python scripts or Jupyter notebooks, and specifies a conda environment that provides details on the software used for the analysis. Version 1.01 of the dms-vep-pipeline was used.

For each specific project (in this case, deep mutational scanning of the BA.1 and Delta spikes) a separate GitHub repository was created that included dms-vep-pipeline as a submodule. The repository for BA.1 is publicly available at github.com/dms-vep/SARS-CoV-2_Omicron_BA.1_spike_DMS_mAbs and the repository for Delta is at github.com/dms-vep/SARS-CoV-2_Delta_spike_DMS_REGN10933 Note how each repository has a configuration file (the config.yaml file), project-specific input data (the data subdirectory), and a top-level Snakemake file (the Snakefile) that runs the analysis. The output of running the pipeline is placed in a results subdirectory, although only key results files are tracked in the GitHub repository since many of them are very large. The pipeline also generates HTML rendering of the key analysis notebooks and result plots, which are available at dms-vep.github.io/SARS-CoV-2_Omicron_BA.1_spike_DMS_mAbs for BA.1 and dms-vep.github.io/SARS-CoV-2_Delta_spike_DMS_REGN10933 for Delta. Looking at these websites is the easiest way to understand the analysis. Note that many of the plots are interactive charts created with Altair (VanderPlas et al., 2018), and readers are encouraged to use the interactive features to better explore the data.

Analysis of PacBio data to link barcodes to spike mutations. To link each barcode to its spike variant, alignparse (Crawford and Bloom, 2019, J. Open Source Softw. 4, 1915) was used to process the PacBio CCSs to determine the barcode and spike mutations for each CCS.

Several quality control steps were performed. First, the synonymous tags introduced at the end of each amplicon during the library preparation were examined to identify CCSs with discordant tags indicative of strand exchange during library preparation: typically <2% of CCSs had discordant tags, indicating a low rate of strand exchange. CCSs with identified strand exchange or out of frame indels were discarded. Next, the empirical accuracy of the CCSs was wacomputed by examining how often CCSs with the same barcode reported the same spike sequence (the exact method used to compute the empirical accuracy is implemented here: jbloomlab.github.io/alignparse/alignparse.consensus.html#alignparse.consensus.empirical_acc uracy). The empirical accuracies were between 0.65 and 0.75, indicating that a fraction of CCSs correctly report the actual mutations. The inaccuracies are due to a combination of sequencing errors, reverse transcription errors, PCR strand exchange, and occasional actual association of the same barcode with different variants in different cells (which can especially arise if the complexity of the initial virus library integrated into cells at single copy is not much higher than the complexity of the final cell library).

Consensus sequences were then built for each barcode with at least three CCSs, using the method implemented at jbloomlab.github.io/alignparse/alignparse.consensus.html #alignparse.consensus.simple_mutconsensus with max_minor_sub_frac and max_minor_indel_frac both set to 0.2. This approach of requiring multiple concordant CCSs to call a consensus is expected to lead to higher accuracy in the final barcode/spike variant linking, and will generally discard barcodes that are not uniquely linked to a single spike variant.

Files containing the final barcode/variant lookup tables and the analysis notebooks with resulting quality control plots are linked to the main HTML pages in the documentation for the BA.1 and Delta experiments as provided in the Data availability section below.

Analysis of Illumina data to count barcodes for each variant in each experiment. For each experiment, the Illumina barcode sequencing was processed with the parser implemented at jbloomlab.github.io/dms_variants/dms_variants.illuminabarcodeparser.html to determine the counts of each variant in each condition. Barcoded variants were only retained for subsequent analysis if their “pre-selection” counts (no-antibody selection for antibody escape experiments, VSV G pseudotyped infections for functional selections) met some minimal count threshold specified in the config.yaml file of the GitHub repos for the BA.1 and Delta spikes. This thresholding removes variants that are expected to have substantial noise due to low counts. Note that a caveat that should be kept in mind is that the actual key bottleneck is expected to usually occur at the stage of infection with the virus library rather than sequencing, since the barcodes are generally sequenced to a depth that greatly exceeds the complexity of the libraries used for the infections. Therefore, although variants with low counts are expected to have more noise, the counts do not enable a quantitative estimate of the actual bottleneck size experienced by each variant.

Files containing the barcode counts and the analysis notebook with resulting quality control plots are linked to the main HTML pages in the documentation for the BA.1 and Delta experiments as provided in the Data availability section below.

Computing functional effects of mutations. To estimate the functional effects of individual mutations, functional scores for each variant from the counts in the VSV G pseudotyped library infection (which should not impose any selection on the spike) were first computed versus the spike-pseudotyped library infected into ACE2 expressing target cells. The functional score for variant v is defined as log₂ ([n^{v}ₚₒₛₜ/n^{wt}ₚₒₛₜ]/[n^{v}ₚᵣₑ/nw^{wt}ₚᵣₑ]) where n^{v}ₚₒₛₜ is the count of variant v in the post-selection (spike-pseudotyped) infection, n^{v}ₚᵣₑ is the count of variant v in the pre-selection (VSV G pseudotyped) infection, and n^{wt}ₚₒₛₜ and n^{wt}ₚₒₛₜ are the counts of all unmutated (wildtype) variants in each condition. Negative functional scores indicate a spike variant is worse at mediating infection than the unmutated spike and positive functional scores indicate a variant is better at mediating infection than the unmutated spike. The distributions of these functional scores are plotted in FIG. 7.

To deconvolve the functional scores for all spike variants (which often contain multiple mutations) into estimates of the effects of individual amino-acid mutations, global epistasis models (Otwinowski et al., 2018, Proc. Natl. Acad. Sci. 115, E7550-E7558) to the variant functional scores were fit, using the models implemented at jbloomlab.github.io/dms_variants/dms_variants.globalepistasis.html with the Gaussian likelihood function. This fitting estimates how each mutation affects an underlying latent phenotype, as well as the shape of the global epistasis function relating the latent phenotype to the observed functional score. The inferred effect of each mutation was also then re-transformed on this latent phenotype through the global epistasis function to estimate its effect on the observed phenotype. This approach provides a way to deconvolve the information in the multi-mutant variants to more accurately estimate the effects of mutations under the assumptions of a global epistasis model (Otwinowski et al., 2018, Proc. Natl. Acad. Sci. 115, E7550-E7558). For final reporting, the average (median) of the estimated functional effect of each mutation across all the replicates and libraries for each different spike (BA.1 or Delta) was taken. FIG. 15A reports the functional effects on the observed (rather than latent) phenotype, as that is a more relevant measure of its expected impact on spike-mediated infection.

Files containing the effects of mutations on both the latent and observed phenotypes for both individual replicates/libraries and averages across them, the analysis notebooks with relevant quality control plots, and interactive plots summarizing the final estimates are linked to the main HTML pages in the documentation for the BA.1 and Delta experiments as provided in the Data availability section below.

Computing antibody escape by mutations. For the antibody selections, the non-neutralized fraction (probability of escape) pᵥ(c) for each variant v at each antibody concentration c for a given antibody as pᵥ(c)=(nᵥ^{c}/S^{c})/(nᵥ⁰/S⁰) where nᵥ^{c} is the count of variant v at antibody concentration c, nᵥ⁰ is the count of variant v in the no-antibody control, S^{c} is the total counts of the neutralization standard at antibody concentration c, and S⁰ is the total concentration of the neutralization standard in the no-antibody control was computed. These values should in principle fall between 0 (variant is completely neutralized by antibody) and 1 (variant is not neutralized by antibody), and in practice, any values measured as >1 to a value of 1 were clipped.

To deconvolve mutation-level escape values from the measured pᵥ(c) values for the variants (which often contain multiple mutations) at multiple concentrations, the approach implemented in polyclonal software package (jbloomlab.github.io/polyclonal/) (Yu et al., 2022, bioRxiv 2022.09.17.508366) was used, constraining the fits to a single epitope (since only monoclonal antibodies were used). This analysis yields a mutation-level escape score for each observed variant (the β_{m,e} values in the nomenclature of the polyclonal package) which will be zero for mutations that have no effect on antibody escape, and >0 for mutations that mediate antibody escape. These are the values plotted in the heat maps shown in the antibody escape figures; the line plots show site-level summaries of these values (e.g., the sum of the escape values at each site). Note that the polyclonal models (Yu et al., 2022, bioRxiv 2022.09.17.508366) can use the escape values inferred from the deep mutational scanning to predict the non-neutralized fraction for arbitrary mutants, and those measurements were correlated with the IC₅₀ values measured by standard neutralization assays in the antibody escape. For all antibodies, there were replicate measurements (multiple libraries, and in some cases technical replicates of the same library), and the final reported values are the average (median) across these replicates.

Files containing the escape values for both individual replicates/libraries and averages across them, the analysis notebooks with relevant quality control plots, and interactive plots summarizing the final estimates are linked to the main HTML pages in the documentation for the BA.1 and Delta experiments as provided in the Data availability section below.

Processed data. The key results from the analysis are stored in the results subdirectory of the GitHub repos for BA.1 (github.com/dms-vep/SARS-CoV-2_Omicron_BA.1_spike_DMS_mAbs) and Delta (github.com/dms-vep/SARS-CoV-2_Delta_spike_DMS_REGN10933). The easiest way to navigate these results is via the HTML documentation at dms-vep.github.io/SARS-CoV-2_Omicron_BA.1_spike_DMS_mAbs and dms-vep.github.io/SARS-CoV-2_Delta_spike_DMS_REGN10933. These pages contain links to the key data files, as well as interactive heat maps of the functional effects of mutations and the effects of mutations on antibody escape. Note that these plots are interactive, and allow you to filter by certain regions of the protein, the number of variants in which a mutation is seen, the maximum magnitude of an effect at a given site, and other relevant parameters.

In the final output files, mutations are numbered in reference-based (Wuhan-Hu-1) spike numbering. The GitHub repos contain files that convert sequential numbering of the BA.1 and Delta spike to reference-based numbering.

The raw PacBio and Illumina sequencing data have been deposited on the NCBI's Sequence Read Archive with BioProject number PRJNA888402 for the Omicron BA.1 data and PRJNA889020 for the Delta data. The PacBio sequencing linking variants to barcodes can be found under BioSample accessions SAMN31220980 for Omicron BA.1 and SAMN31230634 for Delta. The Illumina barcode sequencing can be found under BioSample accessions SAMN31216920 for Omicron BA.1 and SAMN31230628 for Delta.

### Example II Design and Mapping of the Neutralizing Specificity of Human Anti-HIV Serum Using Deep Mutational Scanning

Design of lentivirus vector backbone for HIV Env. The lentivirus backbone used is described in Example 1. See github.com/dmsvep/HIV_Envelope_BF520_DMS_CD4bs_sera/blob/main/plasmid_maps/lentivirus_backbone_plasmids/pH2rU3_Forlnd_mCherry_CMV_ZsGT2APurR.gb for a map of the plasmid containing this backbone. Briefly, the backbone has a repaired 3′ LTR which allows it to be re-rescued after integrating into cells, constitutive expression of ZsGreen and puromycin resistance as selectable markers for infection, and a TRE3G promoter that inducibly expresses HIV Env when the reverse tetracycline transactivator (rtTA) in the 293T-rtTA cells is induced by the presence of doxycycline. A codon optimized sequence of the HIV Env strain BF520.W14M.C2.26,27 was used. See github.com/dmsvep/HIV_Envelope_BF520_DMS_CD4bs_sera/blob/main/plasmid_maps/lentivirus_backbone_plasmids/pH2rU3_Forlnd_BF520.gb for a plasmid map containing the codon optimized BF520 sequence.

Design of mutant libraries containing mostly functional mutants. To choose mutations to include in the mutant libraries based on prior BF520 deep mutational scanning (Haddox et al (2018) doi.org/10.7554/eLife.34420) previously measured effects of all mutations vs the effects of stop codons were compared. Mutations with an effect measured to be more positive than the 0.95 quantile of stop codon effect in the previous deep mutational scanning were retained. Only three stop codons, at sites 100, 200, and 300, were retained so they could be used as controls for selections.

Mutations present in natural HIV sequences were included even if they had negative effects in previous deep mutational scanning since these mutations are tolerable when combined with some other mutations and there was a preference for neutralization selections to include most naturally occurring mutations. The 2018 filtered web alignment of group M HIV-1 sequences without recombinants was downloaded from the Los Alamos HIV sequence database (Kulken et al., (2003) AIDS Rev. 5, 52-61) and used it to identify any mutations relative to BF520 that were present more than once in the alignment. These mutations were retained for the mutant libraries in addition to those chosen above. See github.com/dms-vep/HIV_Envelope_BF520_DMS_CD4bs_sera/tree/main/library_design for the analysis to choose these mutations. See github.com/dms-vep/HIV_Envelope_BF520_DMS_CD4bs_sera/blob/main/library_design/results/IDT_library_df.csv for the retained mutations.

Design of primers for BF520 mutagenesis. See github.com/jbloomlab/TargetedTilingPrimers for the script used to generate primer sequences to make the chosen mutations. This script generates forward and reverse primers for each mutation which mutate that site to the most frequent codon of the desired mutant. Primer pools were ordered as oPools from Integrated DNA Technologies. See github.com/dms-vep/HIV_Envelope_BF520_DMS_CD4bs_sera/tree/main/library_design/results/primers for the primer sequences.

Production of plasmids containing barcoded mutant BF520 sequences. See github.com/jbloomlab/CodonTilingPrimers for a general description of the PCR mutagenesis strategy used. The key difference is that only primers that introduced the targeted amino-acid mutations were ordered. To mutagenize the BF520 sequences, a codon optimized BF520 sequence was first amplified from a plasmid containing the codon optimized BF520 sequence in a lentiviral backbone. See github.com/dms-vep/HIV_Envelope_BF520_DMS_CD4bs_sera/blob/main/plasmid_maps/lentivirus_backbone_plasmids/pH2rU3_Forlnd_BF520.gb for the sequence of this plasmid. The PCR was performed with the following conditions: PCR mix: 18.5 mL H2O, 2.5 mL DMSO (to reduce off-target amplification), 1.5 mL 10 mM forward linearizing primer (VEP_amp_for_long), 1.5 mL 10 mM reverse linearizing primer (lin_rev_BF520), 1 mL 10 ng/mL BF520 template plasmid, and 25 mL 2×KOD Hot Start Master Mix (Sigma-Aldrich, Cat. No. 71842). Cycling conditions: (1) 95 C/2 min (2) 95 C/20 sec (3) 70 C/1 sec (4) 54 C/10 sec, cooling at 0.5 C/sec (5) 70 C/40 sec (6) Return to Step 2 ×19. The amplified, linearized BF520 sequence was gel purified using NucleoSpin Gel and PCR Clean-up kit (Takara, Cat. No. 740609.5) and then purified using Ampure XP beads (Beckman Coulter, Cat. No. A63881) at 1:1 sample to bead ratio. The amplified BF520 sequence was then used in a modification of a previously described PCR mutagenesis technique (Bloom, (2014) Mol. Bio. Evol. 31, 1956-1978). Forward and reverse pools of codon tiling primers for generating specific mutations were generated using github.com/jbloomlab/TargetedTilingPrimers, as described above. In separate PCR reactions, the forward primer pool was used with the reverse linearizing primer, and the reverse primer pool was used with the forward linearizing primer. The conditions for these PCR reactions were as follows: PCR mix: 7.7 mL H2O, 1.5 mL DMSO, 4 mL 3 ng/mL linearized BF520 template, 0.9 mL 10 mM forward or reverse primer pool, 0.9 mL reverse (lin_rev_BF520) or forward (VEP_amp_for_long) linearizing primer, and 15 mL 2×KOD Hot Start Master Mix. Cycling conditions: (1) 95 C/2 min (2) 95 C/20 sec (3) 70 C/1 sec (4) 50 C/20 sec, cooling at 0.5 C/sec (50 70 C/120 sec (6) Return to Step 2 ×9.

After the mutagenic PCRs, a joining PCR was performed using products from the forward and reverse primer pool mutagenic PCRs. The conditions for the joining PCRs were as follows: PCR mix: 4 mL H2O, 4 mL forward primer pool mutagenesis PCR product diluted 1:4 with H2O, 4 mL reverse primer pool mutagenesis PCR product diluted 1:4 with H2O, 1.5 mL 5 mM forward linearizing primer (VEP_amp_for_long), 1.5 mL 5 mM reverse linearizing primer (lin_rev_BF520), and 15 mL 2×KOD Hot Start Master Mix. Cycling conditions: (1) 95 C/2 min (2) 95 C/20 sec (3) 70 C/1 sec (4) 50 C/20 sec, cooling at 0.5 C/sec (5) 70 C/120 sec (6) Return to Step 2 ×19.

The resulting mutagenized BF520 sequences were gel purified and Ampure bead cleaned with a 1:1 product to beads ratio. These mutagenized sequences were then barcoded with random nucleotide barcodes using a PCR with the following conditions: PCR mix: 30 ng joining PCR product, 1.5 mL 5 mM forward linearizing primer (VEP_amp_for_long), 1.5 mL 5 mM reverse barcoding primer (BC_BF520_long), 15 mL 2×KOD Hot Start Master Mix, and fill to 30 mL with H2O. Cycling conditions: (1) 95 C/2 min (2) 95 C/20 sec (3) 70 C/1 sec (4) 50 C/10 sec, cooling at 0.5/sec (5) 70 C/120 sec (6) Return to Step 2 ×9.

The barcoded mutagenized BF520 sequences were gel and Ampure bead purified, and then cloned into a lentiviral backbone containing plasmid as described in in Example I with some modifications as follows. The barcoded mutagenized sequences were first cloned into an earlier version of the lentiviral backbone during system development. The map of the plasmid used can be found at github.com/dms-vep/HIV_Envelope_BF520_DMS_CD4bs_sera/blob/main/plasmid_maps/lentivirus_backbone_p lasmids/pH2rU3_Forlnd_mCherry_CMV_ZsG_NoBC_cloningvector.gb. The plasmid was digested with MluI and XbaI, and then gel and Ampure bead purified. The barcoded mutagenized BF520 sequences and the digested plasmid were eluted into H2O after Ampure bead purification, which can result in higher Hifi assembly efficiency. A 2:1 insert to vector ratio was then used in a 1 hour Hifi assembly reaction using NEBuilder HiFi DNA Assembly kit (NEB, Cat. No. E2621). The Hifi assembly products were Ampure bead purified and eluted into 20 mL of H2O, which can result in a higher electroporation efficiency. 2 ml of the purified HiFi product was used to transform 20 ml of 10-beta electrocompetent E. coli cells (NEB, C3020K). 5 electroporation reactions for a final count of >5 million CFUs per library were performed. This high diversity of barcoded mutants in transformants was a goal to reduce the potential of barcode sharing in virus libraries, which is described elsewhere herein. The transformed cells were plated on LB+ ampicillin plates, incubated at 37° C. overnight, and the plates were scraped the next day to collect the transformants.

The OD600 of the collected bacteria were measured, and the bacteria were diluted to 15 OD600 and used in five separate 5 mL minipreps (QIAprep Spin Miniprep Kit, Cat. No. 27106X4) each, resulting in a total of 200 μg of plasmid being isolated for each replicate library. The rest of the bacteria were spun down in pellets and stored.

At a later stage of system development, the barcoded mutagenized sequences were moved into an improved version of the lentiviral backbone that uses puromycin selection rather than flow cytometry sorting to enrich infected cells when making the integrated mutant library cell lines. The map of this plasmid can be found at github.com/dms-vep/HIV_Envelope_BF520_DMS_CD4bs_sera/blob/main/plasmid_maps/lentivirus_backbone_plasmids/pH2rU3_Forlnd_mCherry_CMV_ZsGT2APurR.gb. Restriction digest and ligation cloning of the library plasmids and the new lentiviral backbone plasmid was used. As an important note for future deep mutational scanning studies, this cloning strategy was not optimal. Since the barcoded mutagenized sequences were drawn from a plasmid pool with relatively limited diversity compared to mutagenic PCR products (a few million unique barcoded sequences vs >>billions of unique barcoded sequences), this cloning imposed an additional unintended bottleneck on the barcoded mutagenized sequence diversity. This meant that the final plasmid pools for each library had lower barcode diversity than intended, resulting in some degree of barcode sharing, described elsewhere herein. In the future, it is advised for similar deep mutational scanning strategies aiming for extremely high plasmid diversity to only clone from highly diverse mutagenic PCR products rather than any pre-existing mutant plasmid pool, which will always be limited in diversity by transformation efficiencies.

To move the barcoded mutagenized sequences into the improved lentiviral backbone, each mutant plasmid pool and the new lentiviral backbone was digested using MluI and XbaI. The mutagenized barcoded inserts were gel extracted and Ampure bead cleaned from the mutant plasmid pools and the cut lentiviral backbone vector, and eluted in Qiagen EB buffer (Cat. No. 19086). T4 DNA ligase (New England BioLabs, Cat. No. M0202S) was then used to ligate the inserts with the vector, using the following conditions: Reaction mix: 2 mL T4 DNA Ligase Buffer (10×), 50 ng Vector DNA, 45.35 ng insert DNA, 1 mL T4 DNA Ligase, and fill with H2O to 20 mL. The reaction was incubated at room temperature for 10 minutes, heat inactivated at 65 C for 10 minutes, and then Ampure bead cleaned and eluted in 20 mL H2O. NEB 10beta cells (New England BioLabs, Cat. No. C3020K) were then electroporated following the protocol (www.neb.com/protocols/0001/01/01/electroporation-protocol-c3020). Five electroporations per library were performed, for a total of 1 million CFUs per library. Again, as a note to future deep mutational scanning studies, the mutant plasmid pool restriction digest and ligation cloning strategy used here along with a transformation bottleneck <5 million CFUs is not recommended due to potential unintended bottlenecking of barcoded mutants.

Production of cell lines storing BF520 mutant libraries. Production of cell line-stored BF520 mutant libraries was performed similarly to previously described in Example I with modifications (FIG. 21B). This process involved the same steps of: 1) production of VSV G pseudotyped lentiviruses carrying the barcoded mutant BF520 sequences, 2) infection of 293T-rtTA cells with the VSV G pseudotyped viruses, and 3) selection for transduced cells using puromycin.

In order to not bottleneck the diversity of barcoded mutants at this step, it was the aim to produce many more VSV G pseudotyped viruses carrying the barcoded mutant BF520 sequences than the eventual desired library sizes of 40,000 barcoded variants. 500,000 293T cells per well were plated in 6 well plates, and 12 wells were transfected for each library. BioT (Bioland Scientific) was used for the transfections, and the manufacturer's recommendations for the protocol and DNA/transfection reagent ratios were followed. Each well was transfected with 1 μg of lentiviral backbone plasmids carrying the barcoded mutagenized BF520 sequences, 250 ng of an HIV Tat expressing plasmid (HDM-tat1b), 250 ng of an HIV Rev expressing plasmid (pRC-CMV_Rev1b), 250 ng of an HIV Gag-Pol expressing plasmid (HDM-Hgpm2), and 250 ng of a VSV G expressing plasmid (HDM_VSV_G). See github.com/dms-vep/HIV_Envelope_BF520_DMS_CD4bs_sera/tree/main/plasmid_maps for maps of these plasmids. The transfection supernatants for each library were pooled 48 hours post-transfection, filtered through a 0.45 mm SFCA syringe filter (Corning, Cat. No. 431220), and stored in 1 mL aliquots at −80 C. These viruses were titrated based on the percent ZsGreen expression of cells infected with dilutions of virus as determined by flow cytometry, as described in Crawford et al. (2020) Viruses 12, 513. doi.org/10.3390/v12050513). This yielded a total of >20 million viruses per library.

These VSV G pseudotyped viruses were used to infect 293T-rtTA cells with the same number of viruses as barcoded mutants that were desired in the final virus libraries. It was the goal to avoid any bottlenecks in the barcoded mutant sequences before this step because recombination of pseudodiploid lentiviral genomes and mutations caused by lentiviral reverse transcription will alter barcode-mutant linkage during this step (See Example I; Hill, et al., (2018) Nat. Methods 15, 271-274; Schlub, et al. (2010) PLoS Comput. Bio. 6. E100766; Jetzt, et al., J. Virol. 74, 1234-1240). There was an attempt to maintain high diversity in the barcoded sequences in prior steps to ensure each barcoded mutant-carrying lentiviral genome would have a unique barcode so that barcodes would not be repeated in infected cells. After this step, each cell in the library storing cell lines will only have one integrated lentiviral genome with one barcoded mutant, so recombination in future steps is not an issue, and mutations caused by reverse transcription in future steps will not alter mutant BF520 expression from these integrants and can be filtered in PacBio sequencing data, described elsewhere herein.

The goal was to infect the 293T-rtTA cells with between 30,000-40,000 variants per library. 500,000 293T-rtTA per well were first plated in ten six well plates. The next day, at the time of infection, the cells per well in several wells were counted. Based on the average count, each well was infected with the amount of infectious units required for a 0.005 multiplicity of infection, for five six well plates per library. Two days later, the actual multiplicity of infection and infectious units per well for each library was determined by determining the percent of infected cells by flow cytometry on ZsGreen expression and back-calculating the infectious units added per well based on that percentage and the average cell count per well at the time of infection. For each library, cells were then pooled from the number of wells required for total infectious units between 30,000-40,000. The pooled cells for each library were plated in a 10 cm plate. Transduced cells were then selected for using puromycin selection since infected cells expressed the puromycin resistance gene from the lentiviral genome while non-infected cells did not. Puromycin was added 24 hours after pooling at 0.75 ug/mL. 48 hours later, the cells were split into three 15 cm dishes per library with 0.75 ug/mL puromycin. 48 hours later, the media was replaced with fresh media plus 0.75 ug/mL puromycin. 48 hours later (a week after pooling), the cells for each library appeared all ZsGreen positive under a fluorescent microscope and were expanded into one five layer flask (Falcon, Cat. No. 353144) per library. 24 hours later, half of the cells per library were frozen in 1 mL aliquots of 5 million cells in tetracycline-negative heat-inactivated fetal bovine serum (Gemini Bio, Cat. No. 100-800) with 10% DMSO, to be used in future virus library generation. The rest of the cells were used to generate mutant virus libraries as described elsewhere herein.

Production of BF520 and VSV G pseudotyped mutant virus libraries. Since each cell in the cell lines produced contained one barcoded BF520 mutant, it was possible to produce genotype-phenotype linked BF520 mutant virus libraries from them (FIG. 21B). This was achieved by plating 100 million cells per flask in two five-layer flasks per library in 150 mL of tetracycline free D10. 24 hours later, each flask was transfected using BioT by using 225 mL of BioT mixed with 7.5 mL of DMEM and a DNA mix containing 50 μg of each lentivirus helper plasmid (Tat, Rev, and GagPol). Env expression was also induced at the time of transfection by adding doxycycline to a final concentration of 100 ng/mL. 48 hours later, the supernatant for each library was filtered through a 0.45 mM SFCA filter (Nalgene, Cat. No. 09-740-44B). The filtered virus was then concentrated using ultracentrifugation with a 20% sucrose cushion at 100,000 g for one hour. The viruses were resuspended in 500 mL of DMEM and were typically around ten million infectious units per mL. These viruses were then stored at −80 C.

VSV G pseudotyped viruses were also generated from the library cell lines to use for PacBio sequencing and as controls for selections on the effects of mutations on BF520 function, described elsewhere herein. This was achieved by plating four million cells per plate in three 10 cm dishes for each library and transfecting each plate 24 hours later using BioT according to the manufacturer's recommendations. For the DNA mix, 2.5 ug of each lentivirus helper plasmid (Tat, Rev, and Gag-Pol) and a VSV G expressing plasmid (four plasmids, 10 ug total DNA) were used per plate. 48 hours later the supernatants for each library were pooled and filtered through a 0.45 mM SFCA filter. Viruses were stored at −80° C.

PacBio sequencing of mutants present in mutant libraries. Long-read PacBio sequencing was used to simultaneously determine the composition of the mutant libraries contained in the library cell lines and link mutants with their random nucleotide barcodes. First, 1 million 293 Ts per well were plated in poly-L-lysine coated six well plates (Corning, Cat. No. 356515). 24 hours later, two wells of cells were infected with 1 million infectious units of +VSVG library virus per well, for each library. Six hours later, the media was removed, cells were washed with PBS, and each well was miniprepped, which isolates unintegrated lentivirus genomes as described previously in Example I (see also Haddox et al PLoS Pathog. 12.e1006114).ach well was miniprepped independently and eluted using 50 mL of EB.

A two-step PCR strategy was then used to amplify the barcoded mutant BF520 sequences for PacBio sequencing, as described in Example 1.23 Briefly, the miniprepped products for each library were split into two short-cycle initial PCRs that attached single nucleotide tags to each end of the amplicon that were unique for each PCR. The products of these initial PCRs were then pooled for each library for longer cycle PCRs to amplify enough DNA for PacBio sequencing. The single nucleotide tags from the initial PCRs then allowed later estimation of the amount of strand exchange that occurred in the longer cycle PCRs based on the frequency of tags found together in PacBio sequences that were from different first round PCRs. The first round of PCR is a low cycle number to minimize the probability of strand exchange during it, and the number of cycles in the second PCR was lowered as much as possible to minimize strand exchange while still generating enough DNA for PacBio sequencing. The conditions used for the first round of PCRs were: PCR mix: 10 mL of miniprep product, 1 mL of 10 mM 5′ nucleotide tagging primer (PacBio_5pri_G or PacBio_5pri_C), 1 mL of 10 mM 3′ nucleotide tagging primer (PacBio_3pri_C or PacBio_3pri_G), 20 mL KOD Hot Start Master Mix, and 8 mL H2O.

Cycling conditions: (1) 95 C/2 min (2) 95 C/20 sec (3) 70 C/1 sec (4) 60 C/10 sec, cooling at 0.5/sec (5) 70 C/60 sec (6) Return to Step 2 ×7 (7) 70 C/60 sec. The PCR products were cleaned with Ampure beads with a 1:1 product-to-beads ratio and eluted into 35 mL of EB. The following conditions were then used for the second round of PCRs: PCR mix: 10.5 mL of first variant tag set round 1 PCR product, 10.5 mL of second variant tag set round 1 PCR product, 1 mL of 10 mM 5′ PacBio round 2 forward primer (PacBio_5pri_RND2), 1 mL of 10 mM 3′ PacBio round 2 reverse primer (PacBio_3pri_RND2), and 25 mL KOD Hot Start Master Mix. Cycling conditions: (1) 95 C/2 min (2) 95 C/20 sec (3) 70 C/1 sec (4) 60 C/10 sec, cooling at 0.5/sec (5) 70 C/60 sec (6) Return to Step 2 ×10 (7) 70 C/60 sec. The PCR products were Ampure bead cleaned, and each eluted into 40 mL of EB. The cleaned products for each library were pooled. Each library pool was then barcoded for PacBio sequencing using SMRTbell prep kit 3.0, bound to polymerase using Sequel∥ Binding Kit 3.2, and then sequenced using a PacBio Sequel lie sequencer with a 20-hour movie collection time. The data were analyzed as described elsewhere herein (PacBio sequencing data analysis).

Barcode amplification for Illumina sequencing of mutants after selections. After the above step using PacBio sequencing to link each mutant and barcode, future experimental steps only require short read sequencing of barcodes to determine changes in variant frequencies across conditions. Barcodes were amplified for sequencing as described in Example I with slight modifications, repeated here. A first round of PCR was used to amplify the barcodes using a forward primer that aligns to the Illumina Truseq Read 1 sequence upstream of the barcode in the lentiviral backbone and a reverse primer that annealed downstream of the barcode and overlapped with the Illumina Truseq Read 2 sequence.

This PCR used the following conditions: PCR mix: 22 mL of miniprepped selection sample, 1.5 mL of 10 mM 5′ Illumina round 1 forward primer (IlluminaRnd1_For), 1.5 mL of 10 mM 3′ Illumina round 1 reverse primer (IlluminaRnd1_rev3), and 25 mL KOD Hot Start Master Mix. Cycling conditions: (1) 95 C/2 min (2) 95 C/20 sec (3) 70 C/1 sec (4) 58 C/10 sec, cooling at 0.5/sec (5) 70 C/20 sec (6) Return to Step 2 ×27.

The PCR products were Ampure bead cleaned with a 1:3 product-to-beads ratio, and then DNA concentration was quantified using a Qubit Fluorometer (ThermoFisher). A second round of PCR was then performed using a forward primer that annealed to the Illumina Truseq Read 1 sequence and had a P5 Illumina adapter overhang, and reverse primers from the PerkinElmer NextFlex DNA Barcode adaptor set that annealed to the Truseq Read 2 site and had the P7 Illumina adapter and i7 sample index. This PCR used the following conditions: PCR mix: 20 ng of round 1 product as determined by Qubit, 2 mL of 10 mM 5′ Illumina round 2 universal forward primer (Rnd2ForUniversal), 2 mL of 10 mM 3′ Illumina round 2 indexing reverse primer (Indexing primers), 25 mL KOD Hot Start Master Mix, and fill to 50 mL total using H2O. Cycling conditions: (1) 95 C/2 min (2) 95 C/20 sec (3) 70 C/1 sec (4) 58 C/10 sec, cooling at 0.5/sec (5) 70 C/20 sec (6) Return to Step 2 ×19.

The DNA concentration of each round 2 PCR product was quantified using Qubit. The samples were pooled at an even ratio, gel purified, and Ampure bead cleaned at a 1:3 sample to beads ratio, and then sequenced using either P2 or P3 reagent kits on a NextSeq 2000. The data were analyzed as described elsewhere herein (Illumina barcode sequencing data analysis).

Selections on effects of mutations on the function of BF520. To measure the effects of mutation on BF520 mediated entry into cells, cells were infected with VSV G and non-VSV G pseudotyped mutant virus libraries separately. To do this, 1 million TZM-bl cells were plated in each well of six well plates. 24 hours later, each well was infected with 1 million infectious units of VSV G or non-VSV G pseudotyped mutant virus depending on the condition. This amount of virus was used because the goal was to use >20× the size of each mutant library during infections so that each barcoded mutant would be present more than once and less likely to be randomly bottlenecked during the selections. During infections, 100 μg/mL DEAE dextran, which improves the infectivity of Env pseudotyped viruses and results in less random bottlenecking of mutants during infections was added. 12 hours after infection, the cells were washed with PBS, miniprepped using a QIAprep Spin Miniprep Kit to isolate unintegrated lentivirus genomes as described with reference to Example I, and eluted into 30 mL of EB. To improve the DNA recovery, the EB was run through the column twice, incubating at 55° C. for five minutes before spinning each time. The eluent was then used in the barcode sequencing prep described above.

Production of VSV G pseudotyped standard viruses for neutralization selections. For each selection using antibodies or sera, a small amount of a separately produced only-VSV G pseudotyped virus pool carrying known barcodes was spiked in to act as neutralization standards by enabling conversion of barcode counts to absolute neutralization values (See FIG. 8A-8D). These viruses were produced exactly as described in Example I. Briefly, 293T-rtTA cells were transduced at a low multiplicity of infection with a pool of lentiviruses carrying a small set of known barcodes but no viral entry protein in their genomes. Transduced cells were selected for using flow cytometry cell sorting on ZsGreen expression, and then standard viruses were produced by transfecting the cells with the lentiviral helper plasmids and a plasmid expressing VSV G. The result of this process was a standard virus pool with known barcodes that was produced in the same manner as mutant libraries but did not contain any viral entry protein mutants.

Selections on effects of mutations on neutralization escape. A goal was to perform antibody and serum selections at concentrations between the IC90-IC99.9 for each antibody and serum. A spread of concentrations in this range was used because it is difficult to estimate IC9X concentrations and a goal was to use a spread of high neutralization levels to fit biophysical escape models. (Yu et al. Virus Evol/8/ (2022) When performing selections using antibodies or serum with the mutant virus libraries, the VSV G pseudotyped neutralization standard viruses were spiked-in to be 0.5-1% of the total infectious units in the virus pool. From this combined virus pool, 1 million infectious units per selection were incubated with antibody or serum at the desired concentration for one hour. After the incubation, the volume of each condition was raised to 2 mL with 100 μg/mL DEAE dextran using D10 with the appropriate amount of DEAE dextran. Each condition was used to infect one well of TZM-bl cells in a six well dish plated at 1 million cells per well 24 hours prior. 12 hours after infection, the cells were washed with PBS, miniprepped, and eluted into 30 mL of EB. To improve the DNA recovery, the EB was run through the column twice, incubating at 55° C. for five minutes before spinning each time. The eluent was then used in the barcode sequencing prep described above.

Validation pseudovirus neutralization assays. Plasmids containing BF520 with mutations used in pseudovirus neutralization assays were ordered from Twist in the HDM plasmid (github.com/dmsvep/HIV_Envelope_BF520_DMS_CD4bs_sera/blob/main/plasmid_maps/viral_entry_protein_expression_plasmids/HDM_BF520.gb). To produce viruses pseudotyped with each BF520 mutant, 500,000 293T cells per well were first plated in six well plates. 24 hours later 1 μg of a ZsGreen and Luciferase expressing lentivirus backbone plasmid (github.com/dmsvep/HIV_Envelope_BF520_DMS_CD4bs_sera/blob/main/plasmid_maps/lentivirus_backbone_plasmids/pHAGE6-wtCMV-Luc2-BrCr1-ZsGreen-W-1247.gb), 250 ng of each lentiviral helper plasmid (Tat, Rev, and Gagpol), and 250 ng of the HDM plasmid expressing the desired BF520 mutant were transfected into each well. The viruses were collected 48 hours later by filtering the supernatant through a 0.45 mm SFCA syringe filter and storing the virus at −80 C.

To titrate these viruses for use in neutralization assays, 25,000 TZM-bl cells were first plated per well in clear bottom, poly-Llysine coated, black-walled 96 well plates (Greiner, Cat. No. 655930). 24 hours later, each mutant BF520 pseudotyped virus was serially diluted and the cells were infected. 48 hours after infection, the Bright-Glo Luciferase Assay System (Promega, E2610) was used to measure relative light units (RLUs) for each dilution. The average RLU/mL for each BF520 mutant was estimated within a linear range based on its dilution curve. Note that this method and the following described neutralization assay are not the same as a typical TZM-bl neutralization assay, since Luciferase expression will be driven from the lentiviral genome of the infecting virus rather than the pre-integrated Tat-driven Luciferase in the TZM-bl cells, as there is will be no Tat expressed from these lentiviruses. This approach was chosen rather than using DEnv HIV pseudoviruses in typical TZM-bl neutralization assays so that there was no chance of the BF520 Env mutants with combinations of escape mutations to CD4 binding site antibodies or sera recombining into full-length replicative HIV.

For neutralization assays, 25,000 TZM-bl cells per well were plated in clear bottom, poly-L-lysine coated, black-walled 96 well plates. 24 hours later, each antibody or sera was serially diluted, and then each dilution was incubated with each mutant BF520 pseudotyped virus for one hour. An equal volume of D10 with DEAE dextran was then added to a final DEAE dextran concentration of 100 ug/mL, and the TZM-bls were infected. 48 hours later, the Bright-Glo Luciferase Assay System was used to measure RLUs for each dilution.

To calculate fraction infectivity, the average background reading of RLUs was subtracted from uninfected cells from each condition, and then the RLU of each antibody or serum dilution was divided by the average RLUs from cells infected by virus that was incubated with media rather than antibodies or sera. The fraction infectivities were used to fit neutralization curves using neutcurve (jbloomlab.github.io/neutcurve/). Fold change IC80 was compared rather than IC50 for interpretation of the neutralization assays because the deep mutational scanning selections were performed at high levels of neutralization (>IC90 for wildtype BF520).

Computational pipeline overview. For analyzing deep mutational scanning of viral entry protein, a common, modular pipeline is used. See github.com/dms-vep/dms-vep-pipeline for this pipeline. For this Example II, version 2.0.1 of dms-vep-pipeline was used. A repository was created for the analyses performed in this Example II. See github.com/dms-vep/HIV_Envelope_BF520_DMS_CD4bs_sera for the repository. This repository includes the main dms-vep-pipeline as well as all of the scripts, notebooks, and settings necessary to recreate the analysis. Some key results files can be found in this repository, but some results files that are too large are not tracked in the online repository. The pipeline also produces HTML rendering of the key analyses and interactive plots. See dms-vep.github.io/HIV_Envelope_BF520_DMS_CD4bs_sera/for these pages. These pages are the best way to explore the analyses and interactive plots of the results.

PacBio sequencing data analysis. Alignparse (jbloomlab.github.io/alignparse/for documentation) was used to analyze the PacBio sequencing data (Jetzt J. et al. Virol. 74 1234-1240 (2000). The PacBio CCSs went through several filtering steps before it was determined which BF520 mutants were linked to which barcodes. First, evidence of strand exchange during the PacBio sequencing prep PCRs was looked for by computing the fraction of CCSs that contained unexpected pairs of single nucleotide tags, such as pairs of nucleotide tags from different round one PCRs or any wildtype nucleotides. These sequences represented just 0. The summed escape scores for each site are the y-axis values displayed in the line plots in each figure and used to color the PDB structures seen in each figure. The individual escape scores for each mutation can be seen in the heatmaps of the linked interactive plots, like the ones seen in FIG. 23B and FIGS. 30A-30B. The models are also able to predict arbitrary inhibitory concentrations for Env mutants, such as an IC50 or IC80 for serum IDC508 against BF520 with mutations T198D and N276D. This is done by determining the effect of each mutation on escape from each epitope in the neutralizing activity of the serum, and then predicting the non-neutralized fraction of virus depending on the degree each epitope's activity is escaped and the contribution of each epitope to the total neutralizing activity (Yu et al. Virus Evol. 8. veac110 (2022)). These predictions were generated for the BF520 mutants used in the neutralization assays depicted in FIG. 23D and FIGS. 27A-D. The fold change in IC80s was chosen for comparison because these values are similar to the level of neutralization used in deep mutational scanning selections. The model was constrained for each antibody to have one epitope, while sera could have up to two epitopes. The mutations were filtered by requiring mutations to be present in at least three unique variants and to have a functional effect above −1.5. See dms-vep.github.io/HIV_Envelope_BF520_DMS_CD4bs_sera/for interactive plots, notebooks detailing the fitting of these models, and PDBs with b-factors containing the escape values for each model.

Quantification and Statistical Analysis. All quantitative analyses were performed using code available at dms-vep.github.io/HIV_Envelope_BF520_DMS_CD4bs_sera/. Modeling of antibody and serum escape was performed using polyclonal software implemented in jbloomlab.github.io/polyclonal/and as described in the computational analysis section and in Yu et al. (Virus Evol. 8. veac110 (2022)). Modeling of functional effects of mutations on Env entry was performed by fitting global epistasis models as described in the computational analysis section using the dms_variants package as implemented at jbloomlab.github.io/dms_variants/dms_variants.globalepistasis.html.

Example Ill Neutralizing specificities for HIV. Broadly neutralizing anti-HIV sera. A set of sera from individuals with HIV was assembled to test if neutralizing specificities could be mapped in a polyclonal context (Schommers, P. et al., Cell 180, 471-489.e22 (2020)). Sera was chosen based on its ability to broadly neutralize a global HIV panel (deCamp, A. J. Virol. 88 2489-2507 (2014) and potently neutralize BF520 pseudovirus. Based on these criteria, four sera collected from individuals in Germany living with HIV were chosen: two with clade B viruses and two with clade D viruses (FIGS. 29A and 29B). Based on the f61 neutralization fingerprinting panel, (Dona-Rose, N. A. et al., PLoS Pthog.13. e1006148) these sera were predicted to be primarily VRC01 like, meaning they target the CD4-binding site (FIG. 29C). Note that all the sera in this study targeted the CD4-binding site because broad sera that neutralized BF520 (which is relatively resistant to V3 antibodies) was chosen; neutralizing human anti-HIV sera can target other epitopes. Importantly, purified IgGs from these sera were used because antiretroviral drugs present in the sera could interfere with lentiviral-based assays.

Neutralization escape maps of serum IDC561 and its constituent antibody 1-18 are similar. Serum IDC561, which was collected from the same individual from whom the broadly neutralizing antibody 1-18 was isolated (Schomers et al. Cell 180, 471-489 (2020), was first analyzed. The antibody was isolated from B cells from the same blood draw date as the serum, suggesting antibody 1-18 is likely present in the serum. Escape from neutralization by antibody 1-18 was mapped alongside the serum in order to compare the escape maps. It has been previously reported that 1-18 and purified IgGs from IDC561 display similar neutralization of a panel of viral strains and mutants, suggesting that neutralization by serum IDC561 is dominated by 1-18. The maps for serum IDC561 and antibody 1-18 generally show neutralization escape at the same sites in Env, although the relative magnitude differs between the serum and antibody (FIGS. 24A-24D and interactive escape maps linked in figure legend). In particular, both the serum and antibody are escaped by mutations around the V1/V2 loop, at b20/b21, and at the b23-V5-b24 structure (FIGS. 24A-24D). Around the V1/V2 loop, the greatest escape from 1-18 is by mutations at site 198 in the middle of the N197 glycosylation motif (FIGS. 24A, 24B, and 24D) and by mutations to sites 202, 203, and 206 (FIGS. 24A, 24B, and 24D). IDC561 is also escaped by mutations at site 198, but mutations at sites 202 and 203 cause more escape for the serum than for 1-18, whereas there is less escape at site 206 for the serum than for 1-18 (FIGS. 24A, 24C, and 24D). At b20/b21, mutations at sites 428-430 escape both 1-18 and IDC561, but the magnitude of this effect is lower for IDC561 than 1-18 (FIGS. 24A-24D). At the b23-V5-b24 structure, mutations to sites 471, 474, and 476 escape 1-18, but only mutations at site 471 strongly escape IDC561 (FIGS. 24A-24D). The escape map for serum IDC561 was substantially more similar to that of antibody 1-18 than another CD4 binding site antibody, 3BNC117,48 as well as the fusion peptide/gp120-gp41 interface-targeting antibody PGT151 (FIG. 24E). This similarity suggests that antibody 1-18, which was isolated from the individual from which serum IDC561 was obtained, contributes substantially to overall neutralization by this serum as suggested by prior studies. 17 However, the fact that the serum IDC561 map does not entirely mirror that of 1-18 shows that other antibodies or members of the same clonal family also contribute to serum neutralization.

Escape maps of other sera show diverse patterns of neutralization specificity Three more CD4-binding-site-targeting sera were next analyzed. The first of these sera, IDC513, was most escaped by mutations in loop D, similar to the well-characterized antibody 3BNC117 (FIGS. 25A-25D and interactive escape maps linked in figure legend), although that antibody was not isolated from this individual. Both 3BNC117 and IDC513 are escaped by mutations in loop D, particularly at site 281 (FIGS. 25A-25D). However, mutations at sites 276 and 278, which knock out the N276 glycan, enhance neutralization by both 3BNC117 and IDC513 (FIGS. 25A-25D). These mutations also sensitize Env to neutralization by 1-18 and IDC561, but not to the same extent (FIG. 24A). Mutations at sites 456, 459, and 471 in the b23-V5-b24 structure also escape both IDC513 and 3BNC117, and there is lower magnitude escape by mutations in and around the CD4 binding loop and other variable loops (FIGS. 25A-25C). Overall the escape map for IDC513 correlates better with 3BNC117 than 1-18 (FIGS. 25D and 25E). Because 3BNC117 and serum IDC513 are from different individuals, neutralizing antibodies in serum IDC513 must have convergently evolved to target similar sites as antibody 3BNC117. Convergent evolution of broadly neutralizing HIV antibodies from the same heavy-chain genes has been observed previously, (Scheid et al, (2011) Science 333, 1633-1637), although the genes encoding the neutralizing antibodies in serum IDC513 are not known. Note that efforts to induce similar antibody specificities form the basis of some vaccine strategies (McGuire, A. T. Curr Opin. HIV AAIDS 14 294-301; Derking, R. et al. J. Int. AIDS So. 24. E25797 (2021). In contrast to IDC513 and IDC561, the escape map of IDF033 reveals a dependence on the N276 glycan for neutralization (FIGS. 26A and 26B, and interactive escape maps linked in the figure legend). Mutations at sites 276 and 278 that ablate the N276 glycan cause by far the greatest escape (FIGS. 26A and 26B). Other mutations in loop D, particularly at site 281, also more weakly escape from IDF033 (FIGS. 26A and 26B). At the b23-V5-b24 structure, mutations at sites 463 and 465 of the N463 glycosylation motif enhance neutralization by IDF033, but the mutation N463S causes escape by shifting the glycosylation motif to N461 (FIGS. 26A and 30A). Other nearby sites also have mutation-specific effects (FIG. 30A). For example, at site S460, only some of the amino-acid changes cause escape (FIG. 30A). Note that the neutralization fingerprinting panel (FIG. 29C) suggests serum IDF033 also has some V3-targeting activity, but this is not apparent in the escape maps likely because BF520 has a relatively high baseline resistance to V3 targeting antibodies (Simonich, C. A. et al., Cell 166, 77-87 (2016)). The escape map for the final serum, IDC508, revealed neutralization escape at two distinct antibody epitopes (FIGS. 26A, 26C, and 26D, and interactive escape maps linked in figure legend). The existence of two epitopes was inferred by fitting the biophysical model (Yu, T., et al., Virus Evol. 8. veac110 (2022)). to the deep mutational scanning measurements and finding that escape in multiply mutated variants was best explained by mutations affecting antibody binding at two distinct regions. Note that the identification of two separate epitopes is crucially enabled by the ability of the deep mutational scanning system to quantify escape by Envs with multiple mutations (Yu. T., et al., Virus Evol. 8. veac110 (2022)). The first IDC508 epitope depends on the presence of the N276 glycan for neutralization and therefore is escaped by mutations at sites 276 and 278, as well as other mutations in loop D, similar to IDF033 (FIGS. 26A and 26C). Neutralization at this first epitope is also escaped by mutations at the b23-V5-b24 structure, also similar to IDF033 (FIGS. 26A-26C and 30B). The second IDC508 epitope mapped mainly to sites around the V1/V2 loop (FIGS. 26A and 26D). Mutations at site 198 cause escape from neutralization at this second epitope, similar to 1-18 and IDC561 (FIGS. 24A, 24C, 24D, 26A, and 26D). Mutations at sites 201, 202, and 203 and in the V2 loop at sites 160-167 also escape at the second epitope, again similar to IDC561 (FIGS. 24A, 26A, and 26D). Therefore, each of the two epitopes targeted by the neutralizing activity of IDC508 resembled the epitope targeted by another serum.

As will be understood by one of ordinary skill in the art, each embodiment disclosed herein can comprise, consist essentially of, or consist of its particular stated element, step, ingredient, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment. A material effect would cause a statistically significant reduction in the ability to detect viral entry protein susceptibility to a selection pressure, such as the ability to evade a therapeutic treatment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials is individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when the application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method of creating a mutational scanning library of variants of a viral protein comprising:
transfecting a population of cells with:
a plurality of viral vectors, each viral vector comprising:
a functional 3′LTR,
an inducible promoter operably linked to a nucleic acid encoding a barcode and a variant of a viral protein; and
a constitutive promoter operably linked to a reporter and a selectable marker;
a pseudo-typing expression plasmid; and
helper plasmids, wherein viral vectors within the plurality have distinct bar codes and encoded variant viral proteins in relation to other viral vectors within the plurality, and wherein the transfecting results in production of pseudo-typed viruses;
infecting cells with the pseudo-typed viruses at a low multiplicity of infection (MOI); and
selecting for infected cells, thereby creating the mutational scanning library of variants of the viral protein.

2. The method of claim 1, further comprising:
inducing expression of the variant of the viral protein in the infected cells; and
transfecting the infected cells with helper plasmids.

3. The method of claim 1, wherein the low MOI results in each infected cell being infected by only one pseudo-typed virus.

4. The method of claim 1, wherein the inducible promoter is a reverse tetracycline-controlled transactivator (rtTA) promoter.

5. The method of claim 1, wherein the selecting comprises administering puromycin.

6. The method of claim 1, wherein the variants of the viral protein comprise viral entry protein variants.

7. The method of claim 1, wherein the variants of the viral protein are selected from severe acute respiratory syndrome coronavirus (SARS-CoV), SARS-CoV-2, Chikungunya, Ebola, Hendra, hepatitis B, hepatitis C, human immunodeficiency virus (HIV)-1, HIV-2, HIV Env, simian immunodeficiency virus (SIV), influenza, Lassa, measles, Middle East respiratory syndrome coronavirus (MERS-CoV), Nipah, Rabies, or respiratory syncytial virus (RSV) viral proteins.

8. The method of claim 1, wherein the variants of the viral protein comprise variants of a viral entry protein selected from SARS-CoV-2 Spike (S), influenza hemagglutinin (HA), HIV envelope (Env), Chikungunya E1 Env, Chikungunya E2 Env, Ebola glycoprotein (EBOV GP), Hendra F glycoprotein, Hendra G glycoprotein, hepatitis B large (L), hepatitis B middle (M), hepatitis B small (S), hepatitis C glycoprotein E1, hepatitis C glycoprotein E2, Lassa virus envelope glycoprotein (LASV GP), measles hemagglutinin glycoprotein (H), measles fusion glycoprotein F0 (F), MERS-CoV Spike (S), Nipah fusion glycoprotein F0 (F), Nipah glycoprotein G, Rabies virus glycoprotein G (RABV G), RSV fusion glycoprotein F0 (F), or RSV glycoprotein G.

9. The method of claim 8, wherein the viral entry protein is the S protein of SARS-CoV-2 or HIV Env.

10. The method of claim 1, wherein the variants of the viral protein comprise viral Gag Pol variants.

11. The method of claim 1, wherein the variants of the viral protein comprise viral Tat variants.

12. The method of claim 1, wherein the variants of the viral protein comprise viral Rev variants.

13. The method of claim 1, wherein the viral vector comprises a retroviral vector.

14. The method of claim 13, wherein the retroviral vector comprises a lentiviral vector.

15. The method of claim 1, wherein the barcode comprises 4 to 30 nucleotides.

16. The method of claim 1, wherein the barcode is located after the stop codon of the variant sequence.

17. The method of claim 1, wherein the population of cells comprises 293T, HEK293T/17, HEK293F, HEK293S, HEK293SGH, EK293FTM, HEK293SGGD, GP2-293, HeLa, HeLa S3, HeLa B, HeLa T4, COS, COS-1, COS-6, COS-M6A, COS-7, A549, MDCK, HepG2, C2C12, THP-1, HUDEP-2, C8161, CCRF-CEM, MOLT, mIMCD-3, NHDF, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, BS-C-1, monkey kidney epithelial, BALB/3T3 mouse embryo fibroblast, 3T3 Swiss, 3T3-L1, 132-d5 human fetal fibroblasts, 10.1 mouse fibroblasts, 3T3, 721, 9L, A2780, A2780ADR, A2780cis, A172, A20, A253, A431, A-549, ALC, B16, B35, BCP-1, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COV-434, CMLT1, CMT, CT26, D17, DH82, DU145, DuCaP, EL4, EM2, EM3, EMT6/AR1, EMT6/AR10.0, FM3, H1299, H69, HB54, HB55, HCA2, Hepa1c1c7, HL-60, HMEC, HT-29, JY, K562, Ku812, KCL22, KG1, KYO1, LNCap, Ma-Mel 1-48, MC-38, MCF-7, MCF-10A, MDA-MB-231, MDA-MB-468, MDA-MB-435, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT2, RenCa, RIN-5F, RMA/RMAS, Saos-2, Sf-9, SkBr3, T2, T-47D, T84, THP1, U373, U87, U937, VCaP, Vero, WM39, WT-49, X63, YAC-1, or YAR cells.

18. The method of claim 1, wherein the transfected population of cells expresses or is exposed to a pro-viral factor.

19. The method of claim 1, wherein the infected cells express or are exposed to a pro-viral factor.

20. The method of claim 1, wherein the transfected population of cells expresses or is exposed to an anti-viral factor.

21. The method of claim 1, wherein the infected cells express or are exposed to an anti-viral factor.

22. A SARS-CoV-2 mutational scanning library as described herein.

23. Use of a mutational scanning library as described herein.

24. A method of identifying mutations in a viral protein that affect sensitivity of the virus to a selection pressure using a mutational scanning library comprising barcoded cells encoding variant viral proteins, wherein the method comprises:
obtaining the mutational scanning library comprising the barcoded cells encoding variant viral proteins, wherein at least 90% of the cells comprise a non-self-inactivating viral vector comprising a single homozygous barcoded variant nucleotide sequence from a set of homozygous barcoded variant nucleotide sequences in the library integrated into the storage cell's genome, wherein the set of homozygous barcoded variant nucleotide sequences collectively encode viral protein variants comprising at least 15 amino acid substitutions at at least 95% of amino acid positions of the viral protein;
transfecting the storage cells with plasmids comprising sequences encoding viral proteins for production of virions;
culturing the transfected storage cells to produce virions, wherein each virion comprises a homozygous barcoded variant nucleotide sequence encoding the viral protein variant;
exposing the virions to the selection pressure;
sequencing barcodes of variant nucleotide sequences from surviving virions; and
linking sequenced barcodes to encoded viral protein variants to identify mutations in each surviving variant relative to a reference under the selection pressure, thereby identifying mutations in the viral protein that affect the sensitivity of a virus to the selection pressure.

25. The method of claim 24, wherein each viral protein variant is expressed.

26. The method of claim 24, wherein the reference is a counterpart viral protein of a wild-type virus, of a parental virus, or of a baseline clinical isolate.

27. The method of claim 24, wherein the selection pressure is a therapeutic compound.

28. The method of claim 27, wherein the therapeutic compound is undergoing pre-clinical development.

29. The method of claim 27, wherein the therapeutic compound is undergoing clinical development.

30. The method of claim 27, wherein the therapeutic compound comprises viral entry and/or fusion inhibitors.

31. The method of claim 27, wherein the therapeutic compound is an antibody, or sera from humans or animals following infection or vaccination.

32. The method of claim 31, wherein the antibody is disclosed herein in relation to SAR-CoV-2 and/or selected from leronlimab (PRO 140), PRO 542, TNX-355 (ibalizumab), human monoclonal IgG1 anti-gp120 antibody b12, polyclonal caprine anti-HIV antibody PEHRG214, anti-HIV antibody PGT121, anti-HIV antibody 3BNC117, anti-RSV G protein monoclonal antibody clone 131-2G, anti-CXCR4 monoclonal antibody clone 12G5 12G5, anti-RSV F protein antibody MAB8582, anti-RSV F protein antibody MAB8581, anti-RSV F protein antibody MCA490, anti-RSV F protein antibody 104E5, anti-RSV F protein antibody 38F10, anti-RSV F protein antibody 14G3, anti-RSV F protein antibody 90D3, anti-RSV F protein antibody 56E11, anti-RSV F protein antibody 69F6, anti-Ebola virus glycoprotein (GP) monoclonal antibody c13C6, anti-Ebola virus glycoprotein (GP) monoclonal antibody c2G4, anti-Ebola virus glycoprotein (GP) monoclonal antibody c4G7, anti-Ebola virus glycoprotein (GP) monoclonal antibody c1H3, LCA60, REGN3051, REGN3048, anti-Lassa virus glycoprotein antibody 37.2D, anti-Lassa virus glycoprotein antibody 8.9F, anti-Lassa virus glycoprotein antibody 19.7E, anti-Lassa virus glycoprotein antibody 37.7H, anti-Lassa virus glycoprotein antibody 12.1F, and Hendra virus neutralizing antibody m102.4.

33. The method of claim 27, wherein the therapeutic compound comprises a small molecule, a protein, a peptide, a polynucleotide, a polysaccharide, an oil, a solution, or a plant extract.

34. The method of claim 27, wherein the selection pressure is selected from heat, cold, low pH, high pH, and a toxic agent.

35. The method of claim 27, wherein the selection pressure is the ability of the virus to enter (i) a host cell of a species or (ii) a cell expressing a receptor protein of a species that is different from the species from which the cell was derived, wherein the ability is not dependent on presence of a functional unrelated viral entry protein.

36. The method of claim 35, wherein the species is human.

37. The method of claim 35, wherein the host cell is derived from human liver, human lung epithelia, or human lung.

38. An HIV Env mutational scanning library as described herein.


SEQUENCE LISTING (29 sequences)

SEQ ID NO: 1 - protein, 18 aa, Betacoronavirus pandemicum
  source 1..18: note = residues 1146-1163 of SARS-CoV-2 spike glycoprotein
DSFKEELDKYFKNHTSPD

SEQ ID NO: 2 - protein, 18 aa, Betacoronavirus
  source 1..18: note = residues 1146-1163 of MERS-CoV spike glycoprotein
IDFQDELDEFFKNVSTSI

SEQ ID NO: 3 - DNA, 19 bp, synthetic construct
  source 1..19: mol_type = other DNA; note = VEP_amp_for
cagccgagccacatcgctc

SEQ ID NO: 4 - DNA, 24 bp, synthetic construct
  source 1..24: mol_type = other DNA; note = 3'rev_lib_LinJoin_KHDC
cggaagagcgtcgtgtagggaaag

SEQ ID NO: 5 - DNA, 85 bp, synthetic construct
  source 1..85: mol_type = other DNA; note = ForInd_AddBC_2; note = wherein n is a, t, g, or c
gcggaactccactaggaacatttctctctcgaatctagannnnnnnnnnnnnnnnagatcggaagagcgtcgtgtagggaaagag

SEQ ID NO: 6 - DNA, 27 bp, synthetic construct
  source 1..27: mol_type = other DNA; note = 5'for_lib_bcing
gcacgcgcagccgagccacatcgctca

SEQ ID NO: 7 - DNA, 85 bp, synthetic construct
  source 1..85: mol_type = other DNA; note = VSVG_BC1
gcggaactccactaggaacatttctctctcgaatctagatactttactactgcacagatcggaagagcgtcgtgtagggaaagag

SEQ ID NO: 8 - DNA, 85 bp, synthetic construct
  source 1..85: mol_type = other DNA; note = VSVG_BC2
gcggaactccactaggaacatttctctctcgaatctagaggaccattgcgacgtaagatcggaagagcgtcgtgtagggaaagag

SEQ ID NO: 9 - DNA, 85 bp, synthetic construct
  source 1..85: mol_type = other DNA; note = VSVG_BC3
gcggaactccactaggaacatttctctctcgaatctagacctagccactagatggagatcggaagagcgtcgtgtagggaaagag

SEQ ID NO: 10 - DNA, 85 bp, synthetic construct
  source 1..85: mol_type = other DNA; note = VSVG_BC4
gcggaactccactaggaacatttctctctcgaatctagaatggagggagtctactagatcggaagagcgtcgtgtagggaaagag

SEQ ID NO: 11 - DNA, 85 bp, synthetic construct
  source 1..85: mol_type = other DNA; note = VSVG_BC5
gcggaactccactaggaacatttctctctcgaatctagatagtgtaaacgccacgagatcggaagagcgtcgtgtagggaaagag

SEQ ID NO: 12 - DNA, 85 bp, synthetic construct
  source 1..85: mol_type = other DNA; note = VSVG_BC6
gcggaactccactaggaacatttctctctcgaatctagaccaacgcgtgaatcgcagatcggaagagcgtcgtgtagggaaagag

SEQ ID NO: 13 - DNA, 85 bp, synthetic construct
  source 1..85: mol_type = other DNA; note = VSVG_BC7
gcggaactccactaggaacatttctctctcgaatctagaatcgtatccatgggtaagatcggaagagcgtcgtgtagggaaagag

SEQ ID NO: 14 - DNA, 85 bp, synthetic construct
  source 1..85: mol_type = other DNA; note = VSVG_BC8
gcggaactccactaggaacatttctctctcgaatctagaggtcacgtgtctatatagatcggaagagcgtcgtgtagggaaagag

SEQ ID NO: 15 - DNA, 34 bp, synthetic construct
  source 1..34: mol_type = other DNA; note = PacBio_5pri_C_tag1
ctagccattcagaggcagccgagccacctcgctc

SEQ ID NO: 16 - DNA, 52 bp, synthetic construct
  source 1..52: mol_type = other DNA; note = PacBio_3pri_G_tag1
cgctcaaccagtacgagccgtaagttatgtaacgcggaactccacgaggaac

SEQ ID NO: 17 - DNA, 34 bp, synthetic construct
  source 1..34: mol_type = other DNA; note = PacBio_5pri_G_tag2
ctagccattcagaggcagccgagccacgtcgctc

SEQ ID NO: 18 - DNA, 52 bp, synthetic construct
  source 1..52: mol_type = other DNA; note = PacBio_3pri_C_tag2
cgctcaaccagtacgagccgtaagttatgtaacgcggaactccaccaggaac

SEQ ID NO: 19 - DNA, 23 bp, synthetic construct
  source 1..23: mol_type = other DNA; note = PacBio_5pri_RND2
ctagccattcagaggcagccgag

SEQ ID NO: 20 - DNA, 33 bp, synthetic construct
  source 1..33: mol_type = other DNA; note = PacBio_3pri_RND2
cgctcaaccagtacgagccgtaagttatgtaac

SEQ ID NO: 21 - DNA, 30 bp, synthetic construct
  source 1..30: mol_type = other DNA; note = IlluminaRnd1_For
ctctttccctacacgacgctcttccgatct

SEQ ID NO: 22 - DNA, 63 bp, synthetic construct
  source 1..63: mol_type = other DNA; note = IlluminaRnd1_Rev3
ctggagttcagacgtgtgctcttccgatctgtccctattggcgttactatgggaacatacgtc

SEQ ID NO: 23 - DNA, 58 bp, synthetic construct
  source 1..58: mol_type = other DNA; note = Rnd2ForUniversal
aatgatacggcgaccaccgagatctacactctttccctacacgacgctcttccgatct

SEQ ID NO: 24 - DNA, 68 bp, synthetic construct
  source 1..68: mol_type = other DNA; note = Indexing primer; note = wherein n is a, t, g, or c
caagcagaagacggcatacgagatnnnnnnnnnngtgactggagttcagacgtgtgctcttccgatct

SEQ ID NO: 25 - skipped

SEQ ID NO: 26 - DNA, 24 bp, synthetic construct
  source 1..24: mol_type = other DNA; note = VEP_amp_for_long
acgcgcagccgagccacatcgctc

SEQ ID NO: 27 - DNA, 26 bp, synthetic construct
  source 1..26: mol_type = other DNA; note = lin_rev_BF520
ggaaagagtgtgatccaactaggcgc

SEQ ID NO: 28 - DNA, 111 bp, synthetic construct
  source 1..111: mol_type = other DNA; note = wherein n is a, t, g, or c; note = BC_BF520_long
tatgtaacgcggaactccactaggaacatttctctctcgaatctagannnnnnnnnnnnnnnnagatcggaagagcgtcgtgtagggaaagagtgtgatccaactaggcgc

SEQ ID NO: 29 - protein, 41 aa, synthetic construct
  source 1..41: note = CTD - cytoplasmic tail of membrane-bound viral protein
MLCCMTSCCSCLKGCCSCGSCCKFDEDDSEPVLKGVKLHYT